(19) United States
(12) Patent Application Publication
(10) Pub. No.: US 2007/0116607 A1
(43) Pub. Date: May 24, 2007

(54) Microsystems that integrate three-dimensional microarray and multi-layer microfluidics for combinatorial detection of bioagent at single molecule level

(75) Inventors: William X. Wang, Iowa City, IA (US); Jun Yi, Iowa City, IA (US); Sheng Ke, Iowa City, IA (US); Maria Halmela, Iowa City, IA (US); Pertti Lahteenmaki, Iowa City, IA (US); Kazuma Kihara, Iowa City, IA (US)

Correspondence Address: MCKEE, VOORHEES & SEASE, P.L.C., 801 GRAND AVENUE, SUITE 3200, DES MOINES, IA, 50309-2721, US

(73) Assignee: PHARMACOM MICROLELECTRONICS, INC., Iowa City, IA (US)

(21) Appl. No.: 11/287,049

(22) Filed: Nov. 23, 2005

(51) Int. Cl. G01N 33/00

(52) U.S. Cl. 422/83; 422/100

(57) ABSTRACT

Stand-alone microsystems adapted for performing combinatorial detection of bioagents at single molecule level wherein the microsystems are featured with three-dimensional microarray and multi-layer microfluidics to thereby provide high throughput screening and high content screening sufficient to allow for substantially real-time performance of the microsystem. Methods for detection of bioagents at a single molecule level or single organism level include providing a reconfigurable microsystem adapted for performing combinatorial detection of bioagents at a single molecule level and reconfiguring the reconfigurable microsystem for various environments.

### CROSS-REFERENCE TO RELATED APPLICATIONS

Although priority is not claimed, the following related applications are hereby incorporated by reference in their entirety: “The system that prevents airplane hijack attempts and enables the safe landing of endangered aircraft”, USPTO Provisional Patent Application No. 60/403,043. filed Sep. 5, 2002; “Encapsulating quantum dots in phospholipid micelles which is directed to target molecules in a living cell”, USPTO Provisional Patent Application No. 60/403,146, filed Sep. 19, 2002; “Performing Fluorescence Resonance Energy Transfer (FRET) for imaging DNA sequencing with high resolution at single base level”, USPTO Provisional Patent Application No. 60/409,062, filed Sep. 29, 2002; “Localizing and tracing signaling pathways of molecules in a living cell”, USPTO Provisional Patent Application No. 60/409,062, filed Sep. 29, 2002; “Performing whole genome scanning in a single cell”, USPTO Provisional Patent Application No. 60/413,001, filed Oct. 11, 2002, “Conducting Fluorescence-Activated Cell Sorting (FACS) for profiling DNA hybridization event at single bacterium/cell level”, USPTO Provisional Patent Application No. 60/413,018, filed Oct. 15, 2002; “Tracking in vivo the programmed steps in apoptosis pathways which are coordinating by a networked group of proteins”, USPTO Provisional Patent Application No. 60/418,302, filed Nov. 7, 2002; “Watchman—a handheld device that can detect multi-array of bioagents in real-time and provide virtually instantaneous results through a wireless network”, USPTO Provisional Patent Application No. 60/431,015, filed Nov. 29, 2002; “The technology that constructs bacteria-based biosensor by pairing a reporter gene with a molecule-sensing component that responds to bacteria detected”, USPTO Provisional Patent Application No. 60/428,959, filed Jan. 16, 2003; “The method that allows single-round DNA sequencing and optional signal amplification”, USPTO Provisional Patent Application No. 60/409,062, filed Apr. 2, 2003; “A method that facilitates single cell-mediated proteomic profiling”. USPTO Provisional Patent Application No. 60/425,757, filed Jun. 15, 2003; “Conducts Fluorescence-Activated Cell Sorting (FACS) for profiling DNA hybridization event at single bacterium/cell level”, USPTO Provisional Patent Application No. 60/426,770, filed Oct. 14, 2003; “Method of using Neuron-Network algorithm to simultaneously track multiple signal resources from hundreds of distinctive pathways”, USPTO Provisional Patent Application No. 60/429,457, filed Nov. 10, 2003.

### BACKGROUND OF THE INVENTION

1. Field of Endeavor The present invention relates to a number of seemingly diverse technologies which may seem unconnected to one not having the benefit of this disclosure. The present invention relates to molecule profiling Microsystems for collecting, detecting, analyzing and reporting multiple chemical and biological agents of interest in a fluid and airborne medium at a real-time utilizing the integrated technologies of microfluidics and microarray and the merged approaches of proteome and genome.

2. State of Technology

Biosensors are defined as analytical devices that combine a biological material (tissues, microorganisms, enzymes, antibodies, nucleic acids etc.) or a biologically-derived material with a physicochemical transducer or transducing microsystem. This transducer can be optical, electrochemical, thermometric, piezoelectric, magnetic or radioactive. Biosensors usually yield a digital electronic signal which is proportional to the concentration of a specific analyte or group of analytes. While the signal may in principle be continuous, devices can be configured to yield single measurements to meet specific application requirements. Biosensors have been used in a wide variety of analytical problems including those found in medicine, the environment, food processing industries, security and defense. The emerging field of bioelectronics seeks to exploit biology in conjunction with electronics in a wider context encompassing, for example, micro or nanoscale biomaterials for information processing, information storage and actuators. A key aspect is the interface between biological materials and electronics since it defines the target, sensitivity, selectivity and speed of the device.

The rapid detection of the pathogens and chemical agents that would be used in a terrorist attack is crucial for developing an appropriate response. In contrast to chemical agents, which must be deployed in substantial amounts, pathogens can be used in very small quantities to elicit an infection as well as widespread fear. Without rapid (seconds to minutes timeframe) detection technology, the first evidence of a biological attack could be widespread sickness in the targeted population. Rapid detection requires some mechanism to amplify a rare, specific biosignature for detection by chemical, microbiological, immunologic, or molecular biological techniques. The polymerase chain reaction (PCR) is widely touted as such a tool, but this requires rigorous sample preparation, complex reactive components of limited shelf life, precise temperature regulation, sophisticated hardware, a complex detection process, and trained personnel. This is appropriate for laboratory diagnosis, but is of limited utility in the field. Further, PCR would be useless in detecting toxic protein exposures. The current widely-used methods of detecting pathogens only achieve sensitivity levels of 5,000 cells per milliliter while the sample is in a prepared solution. In addition, testing can only detect one or two targets at a time and results usually require from eight to 24 hours. Finally, each instrument costs $12,000 to $25,000 and requires lab facilities and several well-trained technologists to run. Newly launched real-time PCR (RT-PCR) instruments can theoretically detect single bacterial cells or viruses within a few minutes but are limited by cost (>$50,000 per instrument), are complicated to operate and must be located in a laboratory setting.

The critical link in most detection systems is to provide a sufficient amount of the material for analysis, or to elicit a distinctive, detectable signal that is responsive to a particular biosignature of the agent. Enzymatic cascades have been shown to elicit thousands-fold amplified responses to the input elicitor. The initiation of most pathogenic responses involves the interaction of the biothreat with a particular cellular receptor. Advantage could be taken of that agent:receptor interaction in a bioengineered complex linked to an amplification cascade, yielding a specific, detectable response by way of a color reaction, light production, electrochemical gradient, etc. To meet this challenge, it is crucial to develop an inspection system with sensitivity, selectivity, ease of operation, and a capability of testing multiple targets simultaneously.

Current methods for bioagent analysis include plaque assays, immunological assays, transmission electron microscopy, and PCR-based testing of viral nucleic acids. These methods have not achieved rapid detection at a single molecule/single bioentity (bacterium or virus) level and often require a relatively high level of sample manipulation that is inconvenient for infectious materials.

Over the past few years, a variety of proteomic techniques have been developed, allowing many thousands of proteins to be studied based on either their relative abundance, or their enzymatic activities. Most of these technologies, however, are based on the traditional protein separation technique, the 2-dimensional gel electrophoresis (2-D GE), which requires downstream instrumentations such as mass spectrometry in order to identify the proteins of interest individually. They are therefore time-consuming and not easily automatable.

Newer technologies, especially those based on microarray platforms, have the potential to rapidly profile the entire proteome, thus are capable of revealing novel protein functions and mapping out comprehensive protein interaction networks of an organism.

The miniaturization of high-throughput screening on a single microscope-sized glass slide has the undeniable advantage of needing only minute quantities of expensive reagents for most biological assays. Nevertheless, the challenges when dealing with proteins are numerous and complex, requiring intricate manipulation and care to ensure preservation of features such as spot uniformity, stable immobilization and preservation of desired protein activity in a microarray.

Typically, chemical/biological sensing is carried out using “extract and evaluate” procedures, where a sample is removed from a certain location and analyzed to determine the components present, both qualitatively and quantitatively, usually with macro equipment in a laboratory situation and with hours of work. This process obviously is time-consuming, limited in application, can be very expensive depending on the difficulty of the extraction process, and especially not fit the biodefense situation or first responder scenario which requires real-time detection, rapid confirmation and instant reaction.

Sample extraction from within a microsystem would require either alternation of the system design to incorporate a sample exit point, or halting the process and opening the unit to remove the sample material. The latter technique would in most cases require destruction of the microsystem, and both processes will cause operational hurdles. With the advent of numerous microscale systems dedicated to biological separation, processing, handling or sensing, this cumbersome process is simply not feasible.

Current detection methods have a number of limitations including large size, the high cost of consumables, limited multiplexing, long analysis times, limited sensitivity and susceptibility to false positives.

Current methods of processing of liquid, or solid or aerosol samples, or a combination of two or three have performance limitations in several spectrums including requirements for extensive manual preparation, requirements for complex fluidics and requirements for large amounts of consumables. The need for effective miniaturized sensors has driven a massive research effort towards this end, with systems varying in both principal of operation and morphology. However, despite recent advances in the field of MEMS-based sensors, the fabrication of miniaturized optical biosensors still tends to be a relatively difficult process, limited largely by complicated device fabrication and packaging.

Optical/electronic biosensors are particularly difficult to fabricate, as coupling into microsystem typically requires accurate alignment components, such as micro-positioning stages for end-fire coupling. Elements such as grating couplers and V-groove couplers may alleviate some of these difficulties, but are challenging and often impossible to integrate into existing Microsystems. A simple method to embed an optical/electronic sensor in an existing biosensor system is an integrated optical waveguide, which can allow light to be effectively conducted to a select point of interest within the device with minimal interference. Applications for this type of optical sensor vary from micro total-analysis systems (μTAS), chemical-sensing within separation channels or miniaturized bioreactors and artificial tissue culture substrates.

Therefore, despite advances in these and other fields problems and obstacles remain.

### BRIEF SUMMARY OF THE INVENTION

Therefore, it is a primary object, feature, or advantage of the present invention to improve over the state of the art.

It is a further object, feature, or advantage of the present invention to provide a method and system that performs detection at the single molecule level.

Yet a further object, feature, or advantage of the present invention is to provide a method and system of detection that integrates microarrays and microfluids.

A still further object, feature, or advantage of the present invention is to provide a method and system of detection that implements the 4S's.

It is a further object, feature, or advantage to provide an integrated system of microarray and microfluidics designed to be able to perform the combinatorial detection of bioagents at single molecule level and from multiple environments.

A further object, feature, or advantage of the system is to provide a microsystem with a dual mode architecture that simultaneously performs both genomic test and proteomic test at a single device and in the process greatly reduces false negative/positive results.

A still further object, feature, or advantage of the present invention is to provide a microsystem with integrated modules that enable the detection of targets at single molecule level or single living object level greatly increases the sensitivity of detection.

Another object, feature, or advantage of the present invention is to provide a microsystem that provides for the dynamical merge of the microarray platform and the microfluidic entities to allow for high throughput screening and high content screening in a microsystem.

Yet another object, feature, or advantage of the present invention is to provide for various types of 3-dimensional compacted sensing elements facilitating a variety of bioagents with distinctive physical attributes and unique chemical potential to be tested in a microsystem.

A further object, feature, or advantage of the present invention is to provide for microsticks-in-column, microspheres-in-chamber, and microspacers-in-column to permit the fast circulation, completed reagent recycling and continuously functioning of the microsystem.

A still further object, feature, or advantage of the present invention is to provide for the fractional separation and parallel sampling of a single cell's content that enables observing functional related cellular entities and metabolic related molecules at a fashion of section plane.

Another object, feature, or advantage of the present invention is apply the strategy of “Big to Small” (e.g. the cascaded process of molecule separation from raw sample to single cell or single molecule, which is step-by-step reduction of non-specific contents) and “Small to Big” (e.g. the cascaded process of signal amplification from weak-level molecule-molecule interaction to medium-level fluorescence generation to high-level optical/electronic conversion, which is a step-by-step increase of specific signals) allow high speed and high efficiency signal detection, signal conversion, signal amplification and signal representation.

Yet another object, feature, or advantage of the present invention is to provide for digitalized sample-collection, target-detection, signal-conversion and data-reporting permit streamline operation and real time performance.

A further object, feature, or advantage of the present invention is to provide for extendibility, flexibility and substitutability that implemented and featured in each of subcomponents of the microsystem permit a wide range of applications of the technology in various environments with different purposes. This innovative technology will have an enormous impact on the way DNA, RNA, protein, bacterial, and viruses and all rest of bioentities are collected, detected, analyzed and reported.

It is a further object, feature or advantage of the present invention to provide an innovation related to (1) dynamic merge of distinctive science fields; (2) architecture of the dual mode system for both genomic test and proteomic test; (3) building blocks of principal components; (4) strategies of self-sampling, preconcentration, fluidization and microflow cytometry; (5) procedures of sensing element fabrication and surface molecule immobilization; (6) miniaturized Laser setup and optic component integration; (7) workflow of signal generation, processing and reporting; (8) implementation of software for general system operation and subcomponent manipulation; (9) extendable applications of the microsystem; and (10) attributes and features of critical microdevices in the microsystem.

Another object, feature, or advantage of the present invention is to provide improved capabilities and advanced functionalities in comparison with the instruments which have been produced for similar purposes by others in the industry.

Yet another object, feature or advantage of the present invention is to provide an implementation of the dual mode of genomic testing and proteomic testing that enables detecting nucleic acid-based samples and protein-based samples in parallel.

Yet another object, feature or advantage of the present invention is to provide an implementation of the microarray-in-microfluidics that enables processing massive quantities of samples in a microenvironment.

Yet another object, feature or advantage of the present invention is to provide an implementation of the multiple sample-intakes and flexible collection arms that enables raw sample processing and real-time performance at various environments.

Yet another object, feature or advantage of the present invention is to provide an implementation of the miniaturized Laser Setup that enables light-weight, portability in various field applications.

Yet another object, feature or advantage of the present invention is to provide a microsystem integrated with four functional modules: biobytes detector, biobytes processor, biobytes reporter and biobytes trigger.

Yet another object, feature or advantage of the present invention is to provide a biobytes detector which places targeted molecules on a microfabricated multidimensional surface with nanometer spatial resolution, and test results can be read in a single image by optical, electrochemical, fluorescent, radioactive, or chemiluminescent methods.

Yet another object, feature or advantage of the present invention is to provide a biobytes processor which interprets data through an incorporated data-mining engine that is coupled with the data channels of intranet, internet or wireless.

Yet another object, feature or advantage of the present invention is to provide a biobytes reporter which visualizes and presents the processed data via the interface of an attached monitor, a remote desktop in a network, or a cellular phone within a few minutes.

Yet another object, feature or advantage of the present invention is to provide a biobytes trigger which initializes a predefined chemical reaction, biological procedure, mechanical motion or human response that results in a desired outcome.

Yet another object, feature or advantage of the present invention is to provide a Living Object-based and Surface Molecule-mediated (LOSM) platform that is particularly designed to capture and detect bioagents at single molecule/single virus/single bacterium level.

Yet another object, feature or advantage of the present invention is to provide for optimal performance of a biodetection system in its speed, sensitivity and selectivity achieved through a system design based on a maximum simulation of the “natural” situation of molecule-molecule interaction; and a maximum imitation of the natural response mechanism, natural molecule complimentary, natural messaging flow, and natural signaling pathway when a target approaches to and interacts with its potential receiver at a molecular, cellular or organic level.

Yet another object, feature or advantage of the present invention is to provide for the bioagent detection and identification of the system to be accomplished through the LOSM platform and not by artificially extracting living objects and forcefully changing their natural attributes.

Yet another object, feature or advantage of the present invention is to provide for using multiple sample collection interfaces, including 1) a mini-syringe for scaled collection of liquid; 2) a mini-pressure hose for intake of air; 3) a mini-screw for penetration of solid matter; 4) a pin-tip for scratch of object surface; 5) extendable pipes that are jointed with the sampling interfaces described as above for extending to different locations within a certain range; 6) preconcentrator: for concentrate particles of interest from a small volume of air; and 7) volumetric container: for concentrate agents of interest from a small volume of liquid.

Yet another object, feature or advantage of the present invention is to use sample fluidizers, where air or solid samples are transformed to liquid phase.

Yet another object, feature or advantage of the present invention is to provide use of sample mixing chambers, where the place that is microspheres interact with fluidized sample molecules.

Yet another object, feature or advantage of the present invention is to provide use of microsphere filters, which selectively permits microspheres with a certain bioaffinity to pass through.

Yet another object, feature or advantage of the present invention is to provide use of U-Turn Pipes, which recycle unbound microspheres after elution.

Yet another object, feature or advantage of the present invention is to provide an implementation of a micro-preconcentrator, micro-fluidizer and micro-thermal generator that enable rapid sample conversions from air or solid phase to liquid.

Yet another object, feature or advantage of the present invention is to provide a implementation of the microsphere-in-microcolumn that enables instructed sample sorting, guided sample separation, directed sample routing, and scheduled sample distribution.

Yet another object, feature or advantage of the present invention is to provide an implementation of the organic light emitting diodes that enables managed conversion from photon to electron or vice versa.

Yet another object, feature or advantage of the present invention is to provide an implementation of the modulated components that enables system expendability and unit substitutability.

Yet another object, feature or advantage of the present invention is to provide a component that is implemented for molecule preparation through cell lysis, DNA extraction, restriction enzymes digestion, fluorescent labeling and further obtain electrophoretic fingerprints.

Yet another object, feature or advantage of the present invention is to provide a component that is implemented for sequencing single molecules of nucleic acid, DNA or RNA, at the rate of one million bases per second by electrophoresis of the charged polynucleotides through a solid-state nanopore channel of molecular dimensions. The nanopore channel with a diameter and length of a few nanometers (10⁻⁹ meters) is made in a silicon-based chip that has nanoelectrodes placed adjacent to the pore. High-speed electronic equipment with exceptional signal acquisition capabilities is used to analyze electronic properties of individual subunits of DNA or RNA in order to obtain the linear composition of each polynucleotide molecule.

Yet another object, feature or advantage of the present invention is to provide a component that is implemented for sending the signal of the polynucleotide sequence to an external viewer and compare the sequence data against an “on-air” genome database through a wireless internet network.

Yet another object, feature or advantage of the present invention is to provide an implementation of the piezoelectric interface to be able to convert cantilever oscillation to energy. Piezoelectric energy generators are implemented for enabling self-powered sensing mechanism.

Yet another object, feature or advantage of the present invention is to provide an implementation of the reactor-coated microsticks, microcantilevers, microtextures and microbranches that enables detection of samples collaboratively with high sensitivity and selectivity.

Yet another object, feature or advantage of the present invention is to provide an implementation of the collaborative reactors that enables precise molecule recognition, less non-specific binding and reduced false positives.

Yet another object, feature or advantage of the present invention is to provide collaborative reactors not to be limited to a group of antibodies or ligands, or those molecules that can bind specifically to the target without displaying significant nonspecific binding with other solution molecules.

Yet another object, feature or advantage of the present invention is to provide a collaborative reactor that can be virtually any molecule that can specifically bind the target without displaying significant nonspecific binding toward other molecules in the solution. It can be a receptor, ligand, antibody, inhibitor or competitor of that target agent, if a unique molecule-molecule interaction, such as a “lock and key” pattern, a stable complex, a strand hybridization, a helix match, or a structural complementary, can be generated through the interaction of the engaged parties and observed as a recognizable binding force, a detectable conformation variance, a measurable energy level change, a readable electric vibration, or a quantifiable light emission.

Yet another object, feature or advantage of the present invention is to provide a collaborative “reactor” having affinity for a target molecule which is covalently attached to an insoluble support and functions as bait for capturing the target from complex solutions.

Yet another object, feature or advantage of the present invention is to provide collaborative reactors which include small organic compounds that are able to dock into binding sites on proteins, inorganic metals that form coordination complexes with certain amino acids in proteins, hydrophobic molecules that can bind nonpolar pockets in biomolecules, proteins with specific binding regions that are able to interact with other proteins, and antibodies, which can be designed to target any biomolecule through their antigen binding sites.

Yet another object, feature or advantage of the present invention is to provide for designing, developing, and implementing a variety of collaborative, intelligent and effective reactors.

Yet another object, feature or advantage of the present invention is to provide for the reactors to be fabricated based on the origin of the cells or bacteria and individuality of the substrains in which narrows down targets.

Yet another object, feature or advantage of the present invention is to provide for the reactors to be fabricated based on the structural uniqueness of the biomarkers to thereby provide high sensitivity reactors.

Yet another object, feature or advantage of the present invention is to provide for the reactors to be fabricated based on the surface molecules of the cells or bacteria in which optimizes the binding conditions of reactors.

Yet another object, feature or advantage of the present invention is to provide for the reactors to be fabricated based on the cell or bacterium-produced proteins, -released toxins and -induced substrates at metabolic pathways, in which designs high selectivity reactors that enable subtypes distinguish.

Yet another object, feature or advantage of the present invention is to provide for the implementation of the semiconductor/optic material-based substrates enables reusability and stability of the sensing elements.

Yet another object, feature or advantage of the present invention is to provide for the first type of microsticks, as the new generation of “microarray”, are able to capture target, execute detection, convert signal and transmit data at once.

Yet another object, feature or advantage of the present invention is to provide for the microsticks are used for detecting living objects based on surface molecule interactions. The collaborative reactors with unique epitopes that attach to surface molecules of living objects are fabricated in outer layer of the thin film optic fiber, that capture spores, bacteria, viruses and large molecule complexes.

Yet another object, feature or advantage of the present invention is to provide for the second type of microsticks to be used for detecting biomolecules based on bioaffinity. The specific mediate molecules such as proteins, oligonucleotides, polysaccharides, lipids, or small peptides are fabricated in outer layer of the thin film optic fiber, that can interact with a variety of biomolecules.

Yet another object, feature or advantage of the present invention is to provide for the third type of microsticks to be used for detecting airborne particles based on weight-caused pressure change. The specific mediate molecules are fabricated in outer layer of the thin film optic fiber, that can interact with a variety of airborne particles.

Yet another object, feature or advantage of the present invention is to provide for the scheduled binding reliever to be implemented as a supportive component in the microsystem designated for capture and detect large particles such as bacteria. There are two types of the scheduled binding Reliever.

Yet another object, feature or advantage of the present invention is to provide for the scheduled binding reliever to be individually but coupled with a sensing element in a close-by environment. It is able to can autonomously disassociate a captured object that attaches to a reactor at surface of a sensing element based on a electrostatic mechanism. The action leaves a space for a new target to approach and bind to the sensing element as the next wave of sample flows in.

Yet another object, feature or advantage of the present invention is to provide for the scheduled binding reliever to be implemented as a part of the sensing element itself, which is able to autonomously disassociate an object that binds to a reactor at surface of the sensing element as the electrostatic stage varies.

Yet another object, feature or advantage of the present invention is to provide for the strategy of microsphere-facilitated biocatalyst which offers well-controlled environment for the reactor-target interaction.

Yet another object, feature or advantage of the present invention is to provide a strategy of microsphere-facilitated bioaffinity which offers an appropriately concentrated environment the reactor-target interaction.

Yet another object, feature or advantage of the present invention is to provide a strategy of microsphere-facilitated hybridization which offers a better interactive surface for the reactor-target interaction.

Yet another object, feature or advantage of the present invention is to provide a Single-Round DNA Sequencer designed to perform single-round DNA sequencing at a microfluidic environment.

Yet another object, feature or advantage of the present invention is to provide a sequencing element where each of the four nucleotides is labeled with four different fluorescent tags and the resulting fluorescent signals with their different wavelengths are converted to specific electronic signals. The cascade of the overall reaction with respect to analysis of DNA consists of the following steps:
(i) The specific DNA fragment of a pathogen gene, which represents a unique region of the target, is selected as the object of analysis; (ii) The single-round replication of the selected DNA region is initialized. The four nucleotides, adenine (A), thymine (T), cytosine (C) and guanine (G) are labeled with fluorescent tags with four different colors, which are green, yellow, red and blue, respectively, as each nucleotide enters the reaction; (iii) The fluorescent tracers, which have four different colors and emit photons with four distinct wavelengths of light; (iv) A photon with a certain wavelength strikes a light-sensitive material and kicks out a single electron which then instigates an avalanche of millions of electrons in a kind of sparking process within a microvacuum tube; (v) Once it is excited by absorption of a photon, the electron can leap onto the terminal of a single-electron transistor, where it “throws the switch” and is detected. The electronic signal can be measured using an nanoscale electron counter.

Yet another object, feature or advantage of the present invention is to provide a sequencing element where the molecular recognition-based electron counter is used to record the number of electrons which corresponds to the wavelength emitted by each fluorescent tracer.

Yet another object, feature or advantage of the present invention is to provide a sequencing element where the electron counter has two components: a capacitor and an electrometer for monitoring. The counter is based on seven nanometer-scale tunnel junctions in series. The counter “pumps” electrons onto the capacitor with an error rate of less than one electron in 10⁸. The electron pumping is monitored with a SET-based electrometer fabricated on the same chip as the pump, with a charge sensitivity better than 10⁻² electrons. The capacitor uses microvacuum as the dielectric, resulting in a frequency-independent capacitance. To operate the ECCS (Electron Counting Capacitance Standard) approximately 100 million electrons are placed, one at a time, on the capacitor. The voltage across the capacitor is then measured, resulting in a calibration of the cryogenic capacitor. The electronic signals are amplified, the signal interpreter reads electronic pulses generated from the fluorescent colors of the labels, and the DNA sequence is determined as the random reading continues.

Yet another object, feature or advantage of the present invention is to provide a sequencing element where the Molecular Matching Pattern Indicator is completely different than the one just described that uses color to read sequences, yet has the same goals.

Yet another object, feature or advantage of the present invention is to provide a sequencing element where the fabrication of each of the complementary structures of potential target DNA sequences in microsections. The number of microsections can be from a few to over 10,000 and each can contain one unique DNA sequence. All microsections are designed to be electronically “excited” when binding of complementary DNA sequences occurs. Once it is excited by the absorption of a photon which is designed to be resulted from a perfect molecular matching, the electron leaps onto the terminal of the single-electron transistor, where the electronic signal is propagated to the Molecular Recognition-based Electron Counter. The Counter will localize the signal on the signal emission “map” that describes the precise locations of each microsection and point out which microsection has been excited. The electronic signal will be amplified to reach a readable level. The target DNA was used in this example in order to describe the technology. However, the general concept and similar technology can be easily extended to other types of molecules in order to identify pathogens and chemical agents.

Yet another object, feature or advantage of the present invention is to provide a merged system of microarray and microfluidics, empowered by a dual mode of genomic and proteomic processing, the general performance of a microsystem is overlapped within four stages in a streamline. 1) Stage of Sample Selection & Collection; 2) Stage of Sample Separation & Diffusion; 3) Stage of Detection & Signaling; and 4) Stage of Data Mining & Reporting.

Yet another object, feature or advantage of the present invention is to provide five optional configurations of sampling-to-signaling performed in the microsystem.

Yet another object, feature or advantage of the present invention is to provide for extracted and separated objects to be routed and diffused into a reaction chamber—special designed microspheres existed in the chamber meet the correspondent objects—the unique binding results in the conformation change of the protein—the light change is detected by the single-molecule fluorescence spectroscopy (SM-FRSP).

Yet another object, feature or advantage of the present invention is to provide for extracted and separated objects to be routed and diffused into a reaction chamber—where thin-film-coated texture immobilized at bottom of the chamber binds the correspondent objects—a LED light penetrating from back of the texture excites the bound object—the refracted evanescent wave is detected.

Yet another object, feature or advantage of the present invention is to provide for extracted and separated objects to be routed and diffused into a reaction chamber—where the thin-film-coated 3D-optic fiber binds the correspondent objects with its surface—a light is projected from a miniature LED placed at the distal end of the fiber and passes through inside it—the refracted evanescent wave is detected at another end.

Yet another object, feature or advantage of the present invention is to provide for the extracted and separated objects to be routed and diffused into a reaction chamber—where the moving microsphere in which the fluorescence reporter dye is conjugated with immobilized molecules executes a quenching reaction while the correspondent object binds with it—the reporter fluorescence is absorbed via fluorescence resonance energy transfer (FRET).

Yet another object, feature or advantage of the present invention is to provide for the extracted and separated objects to be routed and diffused into a reaction chamber—where the thin film-coated microcantilever absorbs the correspondent object with its surface—the electron signal resulting from the unique binding passes through nanowires via a CMOS circuit board—the signal is detected.

Yet another object, feature or advantage of the present invention is to provide technologies of the Single Molecule Measurement to be based on three strategic approaches.

Yet another object, feature or advantage of the present invention is to provide for the step-by-step reduction of non-specific contents using the cascaded process of molecule separation from raw sample to single cell or single molecule.

Yet another object, feature or advantage of the present invention is to provide for the step-by-step increase of specific signals using the cascaded process of signal amplification from weak-level molecule-molecule interaction to medium-level fluorescence generation to high-level optical/electronic conversion.

Yet another object, feature or advantage of the present invention is to provide for the transgene and the transgene product to be double checked through genomic and proteomic approaches of single cell at a dual mode system.

Yet another object, feature or advantage of the present invention is to provide for a microsystem designed to have high sensitivity and able to detect bioagents in a nanomole concentration or at single molecule level.

Yet another object, feature or advantage of the present invention is to provide for a microsystem designed to have high selectivity and able to distinguish sub-strains of a bacteria, sub-forms of a virus; shifted conformations of a protein, and single nucleotide polymorphisms of a DNA domain.

Yet another object, feature or advantage of the present invention is to provide for a microsystem designed to have high accuracy and able to provide notification with extreme low “false-positive” alarms (<0.001%) with “true positive” (99.999%) detection.

Yet another object, feature or advantage of the present invention is to provide for a microsystem designed to have the default setting and able to determine the presence or absence of the pre-selected viruses

Yet another object, feature or advantage of the present invention is to provide for a microsystem designed to have high speed of operation and able to respond to target instantaneously and results at real-time or <2 minutes

Yet another object, feature or advantage of the present invention is to provide for a microsystem designed to have high flexibility of operation and able to operate autonomously, unattended and environment independent.

Yet another object, feature or advantage of the present invention is to provide for a microsystem designed to have high portability: 1) weighs <20 lbs, size <20×8×4 inches; (2) operates by plug-in power and/or battery; (3) adapts transformers that has an output power 15-18 VDC, 1 A max an allow the system to be used in different countries; (4) has the capacity of operating at any location at a “24×7” base.

Yet another object, feature or advantage of the present invention is to provide for a microsystem designed to have a desired substitutability for integration or re-assembly that enables cross-integration of components between different mode series.

Yet another object, feature or advantage of the present invention is to provide for a microsystem designed to have a desired simplicity for handling that requires minimal expertise for operation and maintenance.

Yet another object, feature or advantage of the present invention is to provide for a microsystem designed to have desired adaptability and able to take the advantage of new biomarkers and signature molecules as they become available

Yet another object, feature or advantage of the present invention is to provide for a microsystem designed to have a desired capability for a much larger number of samples to be processed. The size of the machine can be proportionally increased or decreased depending on where the instrument is to be operated and how many individual samples are to be targeted.

Yet another object, feature or advantage of the present invention is to provide for a microsystem operable with minimal supporting infrastructure.

Yet another object, feature or advantage of the present invention is to provide for a microsystem operable in a variety of terrain.

Yet another object, feature or advantage of the present invention is to provide for a microsystem adapted to interface with existing and planned command and control systems.

Yet another object, feature or advantage of the present invention is to provide for a microsystem which is robust and can withstand vehicle transport and environmental extremes.

Yet another object, feature or advantage of the present invention is to provide for a microsystem which is a high throughput device.

Yet another object, feature or advantage of the present invention is to provide for a microsystem which is relatively inexpensive and/or performs tests which are relatively inexpensive.

Yet another object, feature or advantage of the present invention is to provide for a microsystem which is disposable or decontamination-capable.

Yet another object, feature or advantage of the present invention is to provide for a microsystem which is operable for long periods of time with minimal maintenance.

Yet another object, feature or advantage of the present invention is to provide for a microsystem which has long shelf-life.

Yet another object, feature or advantage of the present invention is to provide for a microsystem which is sensitive to civilian population susceptibility.

Yet another object, feature or advantage of the present invention is to provide for a microsystem which has low false positive alarm rates that reflect specific mission requirements.

Yet another object, feature or advantage of the present invention is to provide for a microsystem designed to have a desired capability of performing analysis cycle manually or automatically.

Yet another object, feature or advantage of the present invention is to provide for using a Neural-Network algorithm and parallel signal processor that enables processing up to 200 samples simultaneously.

Yet another object, feature or advantage of the present invention is to provide for using a fiber-optic data link that enables high speed of signaling and reporting.

Yet another object, feature or advantage of the present invention is to provide for using a wireless connection and global database porting interface that enables remote-controllability.

Yet another object, feature or advantage of the present invention is to provide for using scheduling algorithms that enable automatic sample injection, reagent loading, kinetic tuning, and 3D flow-layer switching.

Yet another object, feature or advantage of the present invention is to provide for using an operating platform for the microsystem which is implemented in the microsystem.

Yet another object, feature or advantage of the present invention is to provide for using an embedded data mining engine.

Yet another object, feature or advantage of the present invention is to provide for using an algorithm-driven microflow manipulator is implemented in the microsystem.

Yet another object, feature or advantage of the present invention is to provide for using a neural network combinatorial code board.

Yet another object, feature or advantage of the present invention is to provide for using an internet-enabled wireless communication interface in the microsystem.

Yet another object, feature or advantage of the present invention is to provide for using a microsystem built on top of the 4S-architecture—an universal platform and integrated with flexible components and dynamic subunits, it can be easily converted to be a diagnostic tool for performing high throughput tests on any kind of clinic specimens after replacing sensing elements, modifying sample collection interfaces, switching signal processing mode and changing data output setting.

Yet another object, feature or advantage of the present invention is to provide for using a microsystem built on top of the 4S-architecture—an universal platform and integrated with flexible components and dynamic subunits, it can be easily converted to be a detection tool for performing high throughput tests on any kind of virus or bacteria after replacing sensing elements, modifying sample collection interfaces, switching signal processing mode and changing data output setting.

Yet another object, feature or advantage of the present invention is to provide for using a microsystem built on top of the 4S-architecture—an universal platform and integrated with flexible components and dynamic subunits, it can be easily converted to be an inspection tool for performing high throughput tests on any kind of food including meat, vegetable, egg, milk, drink, beverage, wine and others after replacing sensing elements, modifying sample collection interfaces, switching signal processing mode and changing data output setting.

Yet another object, feature or advantage of the present invention is to provide for using a microsystem built on top of the 4S-architecture—an universal platform and integrated with flexible components and dynamic subunits, it can be easily converted to be a evaluation tool for performing high throughput tests on any kind of drug substances and their immunological responses after replacing sensing elements, modifying sample collection interfaces, switching signal processing mode and changing data output setting.

Yet another object, feature or advantage of the present invention is to provide for using a microsystem built on top of the 4S-architecture—an universal platform and integrated with flexible components and dynamic subunits, it can be easily converted to be a monitoring tool for performing high throughput tests on any kind of environmental agents after replacing sensing elements, modifying sample collection interfaces, switching signal processing mode and changing data output setting.

Yet another object, feature or advantage of the present invention is to provide for using a microsystem built on top of the 4S-architecture—an universal platform and integrated with flexible components and dynamic subunits, it can be easily converted to be a first responder and instant warning tool for performing high throughput tests on any kind of bioterrorism agents after replacing sensing elements, modifying sample collection interfaces, switching signal processing mode and changing data output setting.

Yet another object, feature or advantage of the present invention is to provide for using a microsystem built on top of the 4S-architecture—an universal platform and integrated with flexible components and dynamic subunits, it can be easily converted to be an inspection tool for performing high throughput tests on any kind of toxin or poisons after replacing sensing elements, modifying sample collection interfaces, switching signal processing mode and changing data output setting.

Yet another object, feature or advantage of the present invention is to provide for using a microsystem built on top of the 4S-architecture—an universal platform and integrated with flexible components and dynamic subunits, it can be easily converted to be a measurement tool for performing high throughput tests on any kind of diet substances and their side-effects after replacing sensing elements, modifying sample collection interfaces, switching signal processing mode and changing data output setting.

Yet another object, feature or advantage of the present invention is to provide for using a microsystem built on top of the 4S-architecture—an universal platform and integrated with flexible components and dynamic subunits, it can be easily converted to be an inspection tool for performing tests on any kind of explosives after replacing sensing elements, modifying sample collection interfaces, switching signal processing mode and changing data output setting.

Yet another object, feature or advantage of the present invention is to provide for using a microsystem built on top of the 4S-architecture—an universal platform and integrated with flexible components and dynamic subunits, it can be easily converted to be a monitoring tool for performing high throughput tests on any kind of human, animal, plant smell after replacing sensing elements, modifying sample collection interfaces, switching signal processing mode and changing data output setting.

Yet another object, feature or advantage of the present invention is to provide for using a microsystem built on top of the 4S-architecture—an universal platform and integrated with flexible components and dynamic subunits, it can be easily converted to be a evaluation tool for performing high throughput tests on any kind of transgenic material in plants after replacing sensing elements, modifying sample collection interfaces, switching signal processing mode and changing data output setting.

Yet another object, feature or advantage of the present invention is to provide for using a microsystem built on top of the 4S-architecture—an universal platform and integrated with flexible components and dynamic subunits, it can be easily converted to be an inspection tool for performing high throughput tests on any kind of unfriendly agents in warzone, battlefields or unreachable areas after replacing sensing elements, modifying sample collection interfaces, switching signal processing mode and changing data output setting.

Yet another object, feature or advantage of the present invention is to provide for using a microsystem is built on top of the 4S-architecture—an universal platform and integrated with flexible components and dynamic subunits, it can be easily converted to be an underwater surveillance tool for performing high throughput tests on any kind of agents existed in reservoirs, lakes and oceans after replacing sensing elements, modifying sample collection interfaces, switching signal processing mode and changing data output setting.

Yet another object, feature or advantage of the present invention is to provide for using a microsystem built on top of the 4S-architecture—an universal platform and integrated with flexible components and dynamic subunits, it can be easily converted to be an air surveillance tool for performing high throughput tests on any kind of agents contained in airfow after replacing sensing elements, modifying sample collection interfaces, switching signal processing mode and changing data output setting.

Yet another object, feature or advantage of the present invention is to provide for using a microsystem built on top of the 4S-architecture—an universal platform and integrated with flexible components and dynamic subunits, it can be easily converted to be an inspection tool for performing high throughput tests on any kind of inorganic, organic and biological entities at any area, any situation, and in any environment after replacing sensing elements, modifying sample collection interfaces, switching signal processing mode and changing data output setting.

A still further object, feature, or advantage of the present invention is to provide for a dual-mode (genomic and proteomic) system for detecting transgenic material in plants.

One or more of these and/or other objects, features, or advantages of the present invention will become apparent from the specification and claims that follow.

According to one aspect of the present invention, a molecular profiling microsystem is provided. The molecular profiling microsystem is based on a technology platform that blends five technologies: (i) bioaffinity and biosensing technologies that permit detection on a wide range of molecule-to-molecule interactions; (ii) nanofabrication and microengineering technologies that create sophisticated material-processing and 3-D molecular structures; (iii) integrated microarray and microfluidics technologies that allow high-throughput molecular analysis; (iv) data-network and data-mining technologies that support high volume and high speed genomic/proteomic algorithm-driven data processing; (iv) surface physics and fiber-optic technologies that enable signal conversion between protons, electrons, and energy; (v) bionic technologies that adapt features and functions of living beings for the Microsystems.

The advanced features and functions of the system include: (i) ability to identify threats from near neighbors or other spoofing materials with high confidence; (ii) ability to analyze samples in dirty environments or matrices without requiring external sample preparation steps; (iii) ability to detect all classes of biological threats (bacteria, virus and toxin); (iv) ability to perform multiplexed detection (at least 10 and up to 100 bioagents simultaneously); (v) minimal required operator training; and (vi) no special storage or set-up requirements. The system enables transporting the entire traditional biological detection laboratory to a portable device and offers significant advantages in terms of speed, efficiency, cost, use of small sample sizes and automation.

The innovation is reflected in the following aspects: (1) dynamic merge of distinctive science fields; (2) architecture of the dual mode system for both genomic test a n d proteomic test; (3) building blocks of principal components; (4) strategies of self-sampling, preconcentration, fluidization and microflow cytometry; (5) procedures of sensing element fabrication and surface molecule immobilization; (6) miniaturized Laser setup and optic component integration; (7) workflow of signal generation, processing and reporting; (8) implementation of software for general system operation and subcomponent manipulation; (9) extendable applications of the microsystem; and (10) attributes and features of critical microdevices in the microsystem. This innovative technology will have an enormous impact on the way DNA, RNA, protein, bacterial, and virus and all rest of bioentities are collected, detected, analyzed and reported.

In the present invention, a biodetection microsystem is designed to have the ability to identify threats from near neighbors or other spoofing materials with high confidence; analyze samples in dirty environments or matrices without requiring external sample preparation steps; detect all classes of biological threats (bacteria, virus and toxin); perform multiplexed detection (up to 200 bioagents simultaneously).

The improved capabilities and advanced functionalities in comparison with the instruments which have been produced for similar purposes by rest of the industry include:

(1) Implementing the multiple sample-intakes and flexible collection arms that enables raw sample processing and real-time performance at various environments;

(2) Implementing the micro-preconcentrator, micro-fluidizer and micro-thermal generator that enables rapid sample conversions from air or solid phase to liquid.

(3) Implementing the microarray-in-microfluidics that enables processing massive quantity of samples in a microenvironment;

(4) Implementing the microsphere-in-microcolumn that enables instructed sample sorting, guided sample separation, directed sample routing, and scheduled sample distribution;

(5) Implementing the Neural-Network algorithm and parallel signal processor that enables processing up to 200 samples simultaneously;

(6) Implementing the fiber-optic data link that enables high speed of signaling and reporting

(7) Implementing the organic light emitting diodes that enables managed conversion from photon to electron or vice versa;

(8) Implementing the dual mode of genomic testing and proteomic testing that enables detecting nucleic acid-based samples and protein-based samples in parallel. The microsystem integrates the procedures of sample injection, movement, mixing, reaction, separation and detection on one single platform. The present format of biochips is mostly restricted to microarrays, but the preparation of probes for a microarray requires enormous labor, time and cost. Our design has the capabilities of micropumping with picoliter-delivery, cell preparation, protein and DNA extraction from a single cell, single DNA synthesis, desalting, buffer-exchange, separation medium packing, surface modification of microchannels and so on.

(9) Implementing the reactor-coated microsticks, microcantilevers, microtextures and microbranches that enables detecting samples collaboratively with high sensitivity and selectivity.

(10) Implementing the collaborative reactors that enables precise molecule recognition, less non-specific binding and reduced false positive.

(11) Implementing the miniaturized Laser Setup that enables light-weight, portability and various field applications.

(12) Implementing the semiconductor/optic material-based substrates enables reusability and stability of the sensing elements; the fabricated detective interfaces provide the potential to be stable over a wider temperature range and have a longer shelf life than existing technologies.

(13) Implementing the wireless connection and global database porting interface that enables remote-controllability.

(14) Implementing the modulated components that enables system expendability and unit substitutability.

(15) Implementation of the scheduling algorithms that enables automatic sample injection, reagent loading, kinetic tuning, and 3D flow-layer switching.

(16) Handling nano- to pico-liter solutions inside microchips. Handling of extremely small volumes in ambient conditions is very difficult because evaporation of solvent is very fast and thus quantitative treatment of solutions is almost impossible. Moreover, the information obtained from simple assays using such qualitative methodologies is scant and inadequate for critical screening. The microsystem promises a new paradigm of high-throughput pathogen screening. It provides quantitative analytical information quickly and with similar accuracy and precision to that obtained from laboratory instruments.

(17) Determining the information content in single molecules of genetic material at the speed of 1 base per microsecond. Single-round sequencing and analysis of nucleic acids is revolutionary because no other technique can determine the information content in single molecules of genetic material at the speed of 1 base per microsecond.

(18) Permitting rapid identification and differentiation of single nucleotide polymorphisms. The technology enables rapid genotype identification without the need for a complicated PCR procedure.

(19) Reduces labeling time and increases sensitivity. Conventional detection methods for biochips demand chemical modifications of probes. This target labeling is time consuming, expensive and can also change the levels of targets originally present in a sample. A DNA fragment can be detected through a rapid signal amplification procedure using the Single-round DNA Reader and Molecular Recognition-Based Electron Counter.

(20) The microsystem is designed to have faster recovery times, longer lifetimes, lower drift, better automated calibration, self-diagnostics, automated sample preparation, low cost, and no required reagent additions.

According to another aspect of the present invention, the present invention provides for detection at a single molecule level.

According to another aspect of the present invention, the present invention integrates microarrays and microfluids.

According to another aspect of the present invention, the present invention provides for four stages including the stage of sample selection and collection, the stage of sample separation and diffusion, the stage of detection and signaling, and the stage of data mining and reporting.

According to another aspect of the present invention, the present invention provides for an integrated system adaptable to numerous applications for addition or substitutions of components.

According to aspect of the invention, a microsystem is adapted for performing combinatorial detection of bioagents at a single molecule level wherein the microsystem comprises a microarray component and a microfluidics component integrated with the microarray component to thereby provide high throughput screening and high content screening sufficient to allow for substantially real-time performance of the microsystem. The microarray component and the microfluidics component can be integrated using microsticks-in-column, microspacers-in-column or otherwise to thereby permit rapid circulation, completed reagent recycling and continuous functioning of the microsystem. A sample collection interface is preferably operatively connected to the microfluidics component. The sample collection interface can include one or more of a mini-syringe for scaled collection of liquid, a mini-pressure hose for volumetric breath of air, a mini-screw for penetration of solid matter or a pin-tip for scratching of an object surface. The microsystem can also include a signal processing component having at least one mode of operation and operatively connected to the microarray component. The microsystem may also include a data reporting component operatively connected to the signal processing component. The microsystem may also have a dual mode architecture adapted for simultaneously performing both genomic testing and proteomic testing to thereby reduce false negative and false positive results. The bioagent may be a cell and the microsystem may be adapted to provide for fractional separation and parallel sampling of content of the cell to thereby enable observation of functional related cellular entities and related molecules.

According to another aspect of the invention, a universal platform adapted for performing combinatorial detection of bioagents at a single molecule level is provided. The universal platform may include a sample collection interface for collecting a sample, a microfluidics component operatively connected to the sample collection interface, a microarray component operatively connected to the microfluidics component, a signal processing component having at least one mode operatively connected to the microarray component, and a data reporting component operatively connected to the signal processing component.

According to another aspect of the invention, a microsystem includes a sample selection and collection subsystem, a sample separation and diffusion subsystem operatively connected to the sample selection and collection subsystem, a detection and signaling subsystem operatively connected to the sample separation and diffusion subsystem, and a data reporting subsystem operatively connected to the detection and signaling subsystem.

According to another aspect of the invention, a method for detection of bioagents at a single molecule level or organism level is provided. The method includes providing a reconfigurable microsystem adapted for performing combinatorial detection of bioagents at a single molecule level, and reconfiguring the reconfigurable microsystem for an environment.

According to another aspect of the invention, an apparatus and method for dual mode (genomic and proteomic) detection of transgenic material within a plant is provided. The method includes collecting a sample of the plant using a sample collector, retaining plant cells from the sample while removing portions of the sample which are not plant cells using microfluidics and at least one microarray, and measuring transgenic materials in the plant cells.

According to another aspect of the present invention a microsystem is provided that integrates a three-dimensional microarray and multi-layer microfluidics in order to detect bioagents from various environments at single molecule level in real time. The innovation of the microsystems is demonstrated in many ways, including, without limitation, through (1) merging of distinctive science fields; (2) architecture of a dual mode system for both genomic test and proteomic test; (3) building blocks of principal components; (4) strategies of self-sampling, preconcentration, fluidization and microflow cytometry; (5) procedures of sensing element fabrication and surface molecule immobilization; (6) miniaturized Laser setup and optic component integration; (7) workflow of signal generation, processing and reporting; (8) implementation of software for general system operation and subcomponent manipulation; (9) extendable applications of the microsystem; and (10) attributes and features of critical microdevices in the microsystem.

According to one aspect of the invention, the system includes a dual mode architecture that simultaneously performs both genomic test and proteomic test at a single device greatly reduces false negative/positive results. Where the dual mode architecture is used, a turnplate may be used to perform DNA isolation or protein purification. The turnplate allows jointed and robotic chambers within a single device, to thereby create a streamline interface or jointed workflow between multiple sample collectors in the front-end and sensing elements in the back-end. The problems of cell clumping, adhesion to microchamber walls due to the amphoteric nature of some cell types during separation. The turnplate largely reduces the space for a full-cycle sample process and greatly shorten the distance of sample movement, that allow integration and miniaturization of the dual mode system. The turnplate allows for dynamically coordinating highly collaborative processes which include samples transfer between chambers, wastes removal, reagent injection, solution dilution, and maintenance flow recycling. The turnplate assists in enabling the implementation of multi-layer microfluidics in the microsystem. The turnplate greatly simplifies every step of sample collection, sample digestion, sample sorting, sample purification and sample delivery that is carried on in the microsystem. It also significantly reduces the time, labor and cost occurred in sample processes and allows for digitization of each step, if desired.

According to another aspect of the invention, a system is adapted to capture, purify, detect and visualize single molecules or single organisms can be achieved at a conventional laboratory setting or at a situation in which the instrument of detection is integrated with at least four more separated instruments that perform sample collection, single molecule isolation or single cell sorting, light projection, and signal processing respectively. Such types of setting and processes are highly restricted, very laborious, and extremely expensive. A microsystems of the present invention provides an “all-on-one” design by integrating three-dimensional microarrays and combining electrical, mechanical, chemical, and/or microfluidic approaches, are able to carry out all steps, which include raw simple collection from multiple resources; introduction, mixing, and washing of reagents; subsequent extraction; cascaded isolation of target molecules or organisms; manipulation of molecules or cells; temperature cycling; detection of analytes; single generation and amplification; data processing and visualized reporting; on one single lightweight device that is less than 20 pounds. The Microsystems optionally use pressure, acoustic energy, dielectrophoresis, or electroosmotic flow to exercise precise control over very small volumes of liquids. The result of detection with desired sensitivity and selectivity can be obtained at a real-time with a reduced timeframe, minimum labor, much less reagent and significantly low cost.

The Microsystems may collect, isolate and detect single cells mainly based on surface properties of targeted cells. Parameters that may be utilized for detection and identification of single cells include (i) surface properties that appeared in nature physiological conditions; (ii) surface properties that altered by processes of differentiation; (iii) surface properties that after exposure to antibiotics or chemical preservatives; and (iv) surface properties that induced by deliberately applied stimulates which “synchronize” targeted cells.

The microsystem may implement a strategy described as the “induced molecule expression and directional cellular synchronization” for isolating and detecting molecules or organisms of interest. The target molecules in organisms or the target organisms themselves are deliberately induced or stimulated in order to reach a “timing expression” or “calculated synchronization”. This kind of default-setting facilitates the microsystem to precisely determine existence or not of targets.

According to another aspect of the invention artificial nerve terminals (ANT) are provided which are designed to mimic features and functions of the mammalian nerve terminals. The artificial nerve terminals have the capability of collecting, sorting, purification and routing single molecules or single organisms within its terminals in real-time. The artificial nerve terminal may comprise eight components which are situated at four nodes. The first node serves as detection tip and aspiration hose. It contains two types of tips. The tip is coated with thin-film membrane and proper reactors that directly interfaces with plant liquid. Nanowire that has sensitive conductivity is implemented under the membrane. Many tips which are made up with different reactors can be used to target different objects or a same object in a time sequence. The second tip looks like a microcapillary that sucks small quantity of liquid sample within a distance. Nanowire that has sensitive conductivity is joined with each nodes of the polymers. Many tips which are made up with different filtering polymers can be used to obtain different qualities of liquid samples.

The second node of the ANT serves as sample filter and flow cascade. The second node is formed as a branch of extendable and flexible pipes. It contains two types of pipes: (1) the pipe in which the two-way optical fibers lie and the light from projected from the laser station goes through one line of optical fibers and brings back signals from the reactor-coated tip through another line of optical fibers; (2) the pipe in which samples with distinctive physiochemical properties are filtered through polymers, carried by different groups of microspheres and transferred from the capillary tip to another direction based on mechanical, optical, or electrokinetic forces which is involved subsequentially following phases of the movement. Samples are neutralized, digested, step-by-step eliminated in cascaded polymer sections and targeted analytes reach their destination where the reactions of biocatalyst, bioaffinity or hybridization occur.

The third node of the ANT serves as a laser Station and an electric center: They are two stand-alone units but bridged together through an interface. The laser station projects laser light through optical fibers to the tips and carries scattered lights back to the station. The electric center monitors events that the nanowire network has encountered and filters signals at the center.

The fourth node of the ANT serves as a message reader and signal transmitter. The message reader displays the signals right at the handle. The signal transmitter transfers the signals between the device and remote databases through wireless communication.

### BRIEF DESCRIPTION OF THE DRAWINGS

FIG. 1 is a diagram illustrating one embodiment of the technology building blocks of the Microsystems.

FIG. 2 is a diagram illustrating one embodiment of the architecture of the 4-D System

FIG. 3 is a diagram illustrating a dual-mode molecule profiling microsystem according to one embodiment of the present invention.

FIG. 4 is a diagram illustrating one embodiment of a clinic diagnostics microsystem according to the present invention.

FIG. 5 is a diagram illustrating one embodiment of an E. coli detection microsystem according to the present invention.

FIG. 6 is a diagram illustrating another embodiment of an E. coli detection microsystem according to the present invention.

FIG. 7 is a diagram illustrating one embodiment of a virus identification microsystem of the present invention.

FIG. 8 is a diagram illustrating one embodiment of a food inspection microsystem.

FIG. 9 is a diagram illustrating one embodiment of a pharmaceutical screening microsystem.

FIG. 10 is a diagram illustrating one embodiment of an aerosol monitoring microsystem for interior usage.

FIG. 11 is a diagram illustrating one embodiment of an aerosol monitoring microsystem for exterior usage.

FIG. 12 is a diagram illustrating one embodiment of an odorant detection microsystem.

FIG. 13 is a diagram illustrating one embodiment of a poison detection microsystem.

FIG. 14 is a diagram illustrating one embodiment of a diet measurement microsystem.

FIG. 15 is a diagram illustrating one embodiment of an explosives detection microsystem.

FIG. 16 is a diagram illustrating one embodiment of a human smell detection microsystem.

FIG. 17 is a diagram illustrating one embodiment of a forensic detection microsystem.

FIG. 18 is a diagram illustrating one embodiment of a GMO detection microsystem.

FIG. 19 is a diagram illustrating one embodiment of a warzone inspection microsystem.

FIG. 20 is a diagram illustrating one embodiment of an unattended monitoring system.

FIG. 21 is a diagram illustrating one embodiment of an underwater surveillance microsystem.

FIG. 22 is a diagram illustrating one embodiment of an open environment surveillance microsystem.

FIG. 23 is a diagram illustrating one embodiment of a 3-Dimensional compacted microarray microsystem.

FIG. 24 is a diagram illustrating one embodiment of an object capture and object reliever.

FIG. 25 is a diagram illustrating one embodiment of an array of reactor-coated microcantilevers.

FIG. 26 is a diagram illustrating one embodiment of an array of reactor-coated microsticks.

FIG. 27 is a diagram illustrating one embodiment of an array of reactor-coated microbranches.

FIG. 28 is a diagram illustrating one embodiment of an array of reactor-coated microrods.

FIG. 29 is a diagram illustrating one embodiment of an array of reactors immobilized on microtexture.

FIG. 30 is a diagram illustrating one embodiment of interspacers-in-microcolumn.

FIG. 31 is a diagram illustrating one embodiment of the fabrication of microsticks.

FIG. 32 is a diagram illustrating one embodiment of a fiber optic data link.

FIG. 33 is a diagram illustrating one embodiment of a design for a protein microarray plate.

FIG. 34 is a diagram illustrating multiple detective interfaces for microsticks.

FIG. 35 is a diagram illustrating various optional configurations of light sources in a microsystem.

FIG. 36 is a diagram illustrating an overview of MAIDS (Microfabricated Affinity-based Imprint-polymerized Data-mining empowered Sensing) technology.

FIG. 37 is a diagram illustrating one embodiment of a neural-network combinatorial code board.

FIG. 38 is a diagram illustrating single-round sequence reading and signal generation.

FIG. 39 is a diagram illustrating one embodiment of a molecular matching pattern indicator.

FIG. 40 is a diagram illustrating one embodiment of a miniaturized laser setup.

FIG. 41 is a diagram illustrating an integrated IDAT and T7RP-SRLPQ assay.

FIG. 42 is a diagram illustrating stages of detection.

FIG. 43 is a diagram illustrating a turnplate for DNA extraction or protein purification at a microscale without a centrifuge.

FIG. 44 is a diagram illustrating one embodiment of artificial nerve terminals.

### DETAILED DESCRIPTION OF THE PREFERRED EMBODIMENT

Referring now to the following detailed information, and to incorporated materials; a detailed description of the invention, including specific embodiments, is presented.

Unless otherwise indicated, numbers expressing quantities of ingredients, constituents, reaction conditions and so forth used in the specification and claims are to be understood as being modified by the term “about.” Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical value, however, inherently contains certain errors necessarily resulting from the standard deviation found in their respective testing measurements.

Currently, capture, purification, detection and visualization of single molecules or single organisms can be achieved at a conventional laboratory setting or at a situation in which the instrument of detection is integrated with at least other four more separated instruments that perform sample collection, single molecule isolation or single cell sorting, light projection, and signal processing respectively. Those types of setting and processes are highly restricted, very laborious, and extremely expensive. The present invention provides for microsystems designed to be all-in-one in that they are able to carry out all those steps, which include raw simple collection from multiple resources; cascaded isolation of target molecules or organisms; detection and analytes; single generation and data processing; visualization and report; on one single lightweight device that is less than 20 pounds. The result of detection with desired sensitivity and selectivity can be obtained at a real-time with minimum labor and cost.

The present invention provides an integrated system of microarray and microfluidics that is able to perform the combinatorial detection of bioagents within a nanomole concentration or at single molecule level from multiple environments. The present invention contemplates numerous embodiments and numerous variations. A number of diverse embodiments are presented throughout this description. One skilled in the art, having the benefits of this disclosure, will understand the flexibility of the present invention and its numerous applications. It should also be appreciated that the present invention contemplates many different configurations of different subsystems depending upon the particular environment a microsystem is used in and the particular type of sensing required. It is to be understood that different configurations and different subsystems described herein can be combined in different ways and such combinations are also well within the spirit and scope of the invention even though not expressly delineated.

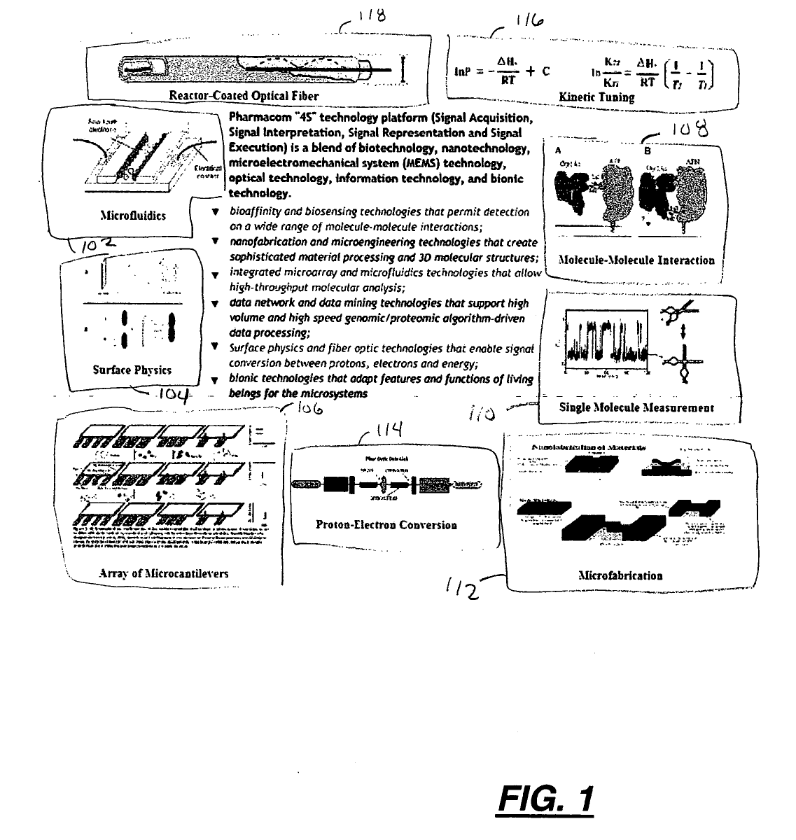

FIG. 1 illustrates various building blocks or subsystems of the present invention, representing different technologies that are used to implement various systems and methods of the present invention. This collection 100 of building blocks or subsystems includes microfluidics 102, surface physics 104, an array of microcantilevers 106, proton-electron conversion 114, microfabrication 112, single molecule measurement 110, molecule-molecule interaction 108, kinetic tuning 116, and reactor-coated optical fiber 118.

The present invention includes a microsystem designed and implemented by combining technologies such as those shown in FIG. 1. The “4S” technology platform of the present invention is a blend of biotechnology, nanotechnology, microelectromechanical system (MEMS) technology, optical technology, information technology, and bionic technology. For example, bioaffinity and biosensing technologies are used to provide detection on a wide range of molecule-molecule interactions. Nanofabrication and microengineering technologies are used to create sophisticated material processing and 3D molecular structures. Integrated microarray and microfluidics technologies are used to allow high-throughput molecular analysis. Data network and data mining technologies are used to support high volume and high speed genomic/proteomic algorithm-driven data processing. Surface physics and fiber optic technologies are used which enable signal conversion between protons, electrons and energy. Bionic technologies are used that adapt features and functions of living beings for the Microsystems.

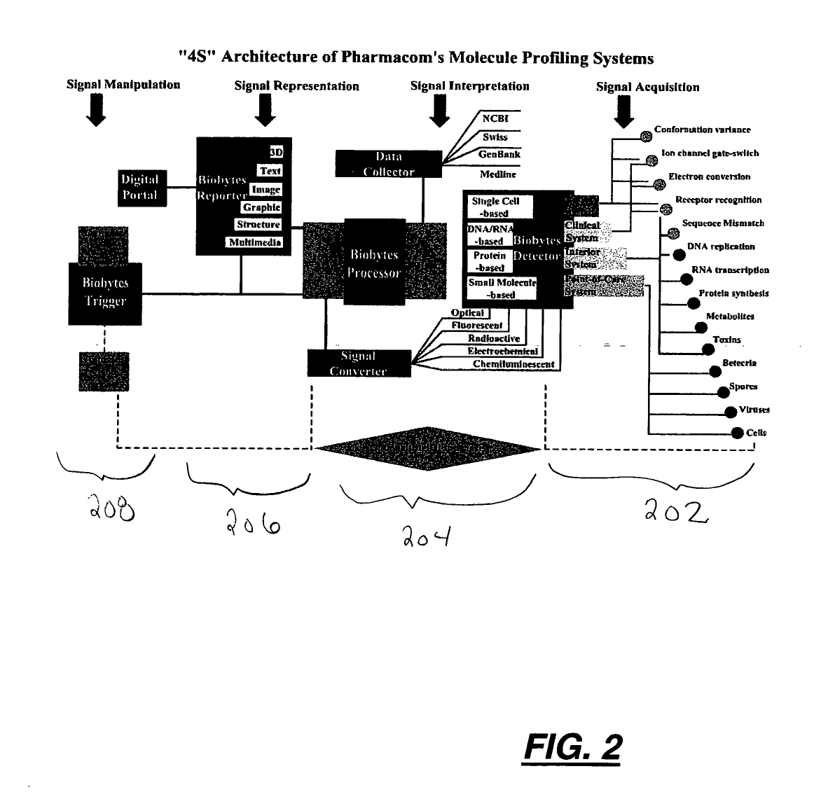

FIG. 2 illustrates an architecture associated with a molecule profiling system 200 according to one aspect of the present invention. Four stages are shown and the system is sometimes referred to as a 4-D system. These stages or dimensions if the system 200 include signal acquisition 202, signal interpretation 204, signal representation 206, and signal manipulation 208. In one embodiment of the present invention, the microsystem is integrated with four functional modules: (i) the biobytes detector places targeted molecules on a microfabricated multidimensional surface with nanometer spatial resolution, and test results can be read in a single image by optical, electrochemical, fluorescent, radioactive, or chemiluminescent methods; (ii) the biobytes processor interprets data through the incorporated data-mining engine that is coupled with the data channels of intranet, internet or wireless; (iii) the biobytes reporter visualizes and presents the processed data via the interface of an attached monitor, a remote desktop in a network, or a cellular phone within a few minutes; and (iv) the biobytes trigger initializes a predefined chemical reaction, biological procedure, mechanical motion or human response that results in the desired outcome.

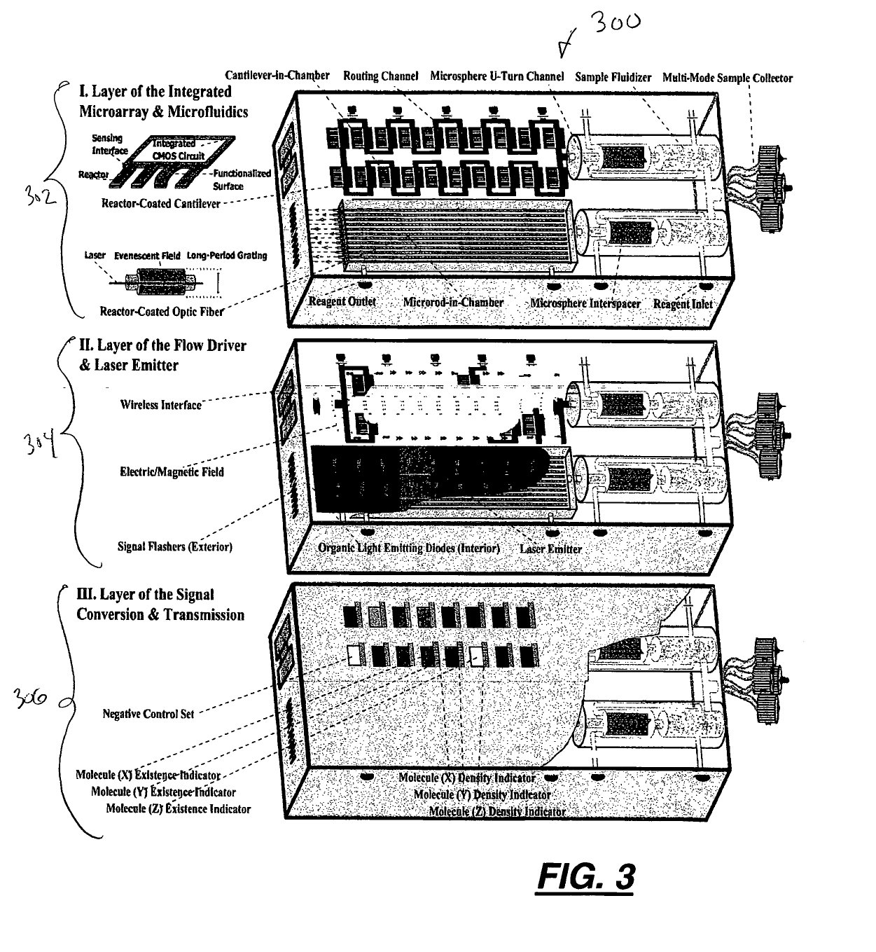

FIG. 3 illustrates a dual-mode molecule profiling microsystem according to one aspect of the invention. As shown in FIG. 3, there are three layers. The first of the layers is the layer of the integrated microarray and microfluidics 302. There is a second layer of the flow driver and laser emitter 304. The third layer 306 is the layer of the signal conversion and transmission. The microsystem shown in FIG. 3 enables targeted DNA, RNA, protein, bacterial, and viruses and all rest of bioentities to be collected, detected, analyzed and reported. It visualizes the 4S technology platform and innovation concepts that are reflected from many aspects: (1) architecture of the dual mode system for both genomic test and proteomic test simultaneously; (2) building blocks of principal components; (3) strategies of self-sampling, preconcentration, fluidization and microflow cytometry; (4) implementation of various types of sensing elements; (5) miniaturized Laser setup and optical component integration; (6) workflow of signal generation, processing and reporting; (7) high extendibility, flexibility, substitutability and portability of the micro system.

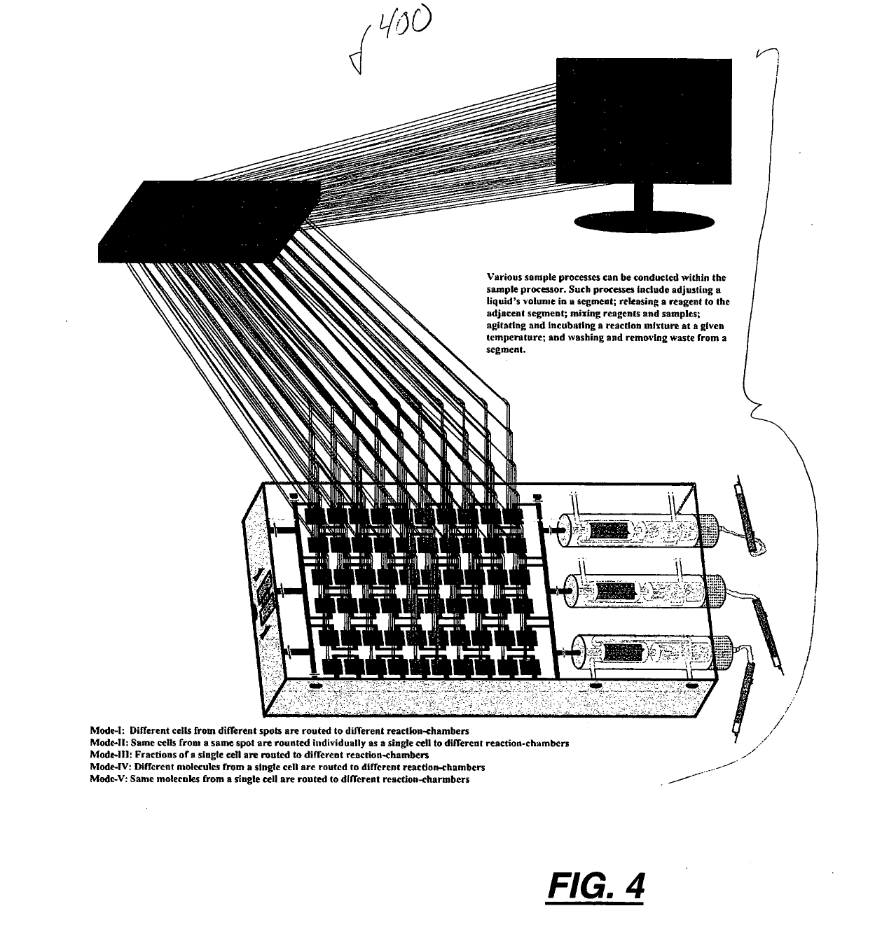

FIG. 4 illustrates one embodiment of a clinic diagnostic microsystem 400 according to one embodiment of the present invention. The microsystem of FIG. 4 can be designed to monitor and stage breast cancer, ovarian cancer, prostate cancer, liver cancer, lung cancer or leukemia through a combinatorial detection based on both genomic testing and proteomic analysis of signature molecules and biomarkers.

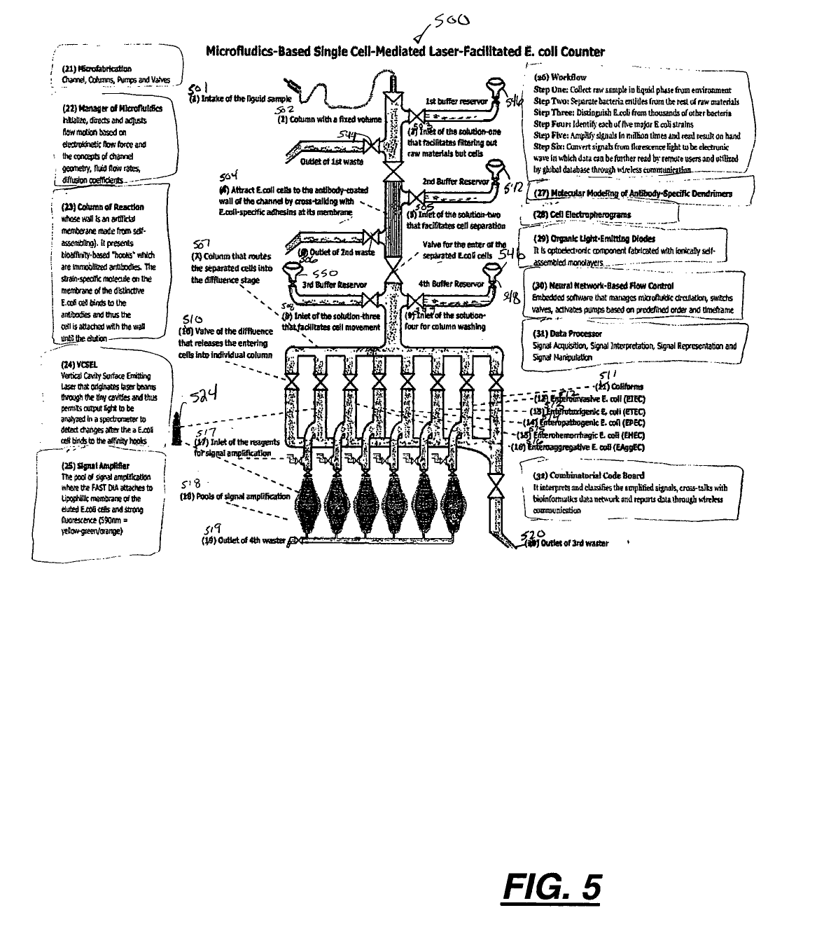

FIG. 5 illustrates one embodiment of an E. coli detection microsystem of the present invention. The workflow of the microsystem 500 has six steps these include (1) collecting a raw sample in liquid phase from the environment, (2) separating bacteria entities from the rest of the raw materials, (3) distinguishing the E. coli from thousands of other bacteria, (4) identifying each of five major E. coli strains, (5) amplifying signals on the order of millions of times and reading results on hand, and (6) converting signals from fluorescence light to an electronic signal in which data can be further read by remote users and used by a global database through wireless communication.

As shown in FIG. 5, intake if a liquid sample occurs at an intake 501. The sample flows to a column 502 with a fixed volume. There is an inlet 503 of a solution that facilitates filtering out raw materials from the cell. The solution flows into the inlet 503 from a first buffer reservoir 540. There is also an outlet 544 for waste materials. There is an inlet 505 of a solution that facilitates cell separation. The solutions flows into the inlet 505 from a second buffer reservoir 542. Next, the antibody-coated wall 504 of the channel attract E. coli cells by cross-talking with E. coli specific adhesions at its membrane. A second outlet 506 is provided for the second waste. A valve 546 is provided to control entry of the separated E. coli cells. An inlet 508 is provided for a third solution that facilitates cell movement and inlet 509 is provided for a fourth solution which provides for column washing. The third solution may be provided from a third buffer reservoir 550 and the fourth solution may be provided from a fourth buffer reservoir 548.

A number of valves 510 are provided that releases the entering cells into individual columns. Coliforms 511 are present in the individual columns. These include enteroinvasive E. coli (EIEC) 512, enterotoxigenc E. coli (ETEC) 513, enteropathogenic E. coli (EPEC) 514, entereohemorrhagic E. coli (EHEC) 515, and Enteroaggregative E. coli (EAggEC) 516.

There are inlets 517 for the reagents for signal amplification. Pools of signal amplification or signal amplifiers 518 are also shown. The pools of signal amplification 518 are where the FAST DIA attaches to the Lipophillic membrane of the eluted E. coli cells and strong fluorescence (such as 590 nm=yellow-green/orange). A combinatorial code board can be used for interpreting and classifying the amplified signals, cross-talks with bioinformatics data network and reporting stat through wireless communication. An outlet 519 for waste is also provided. There is also an outlet 520 for waste.

It should be understood that microfabrication techniques can be used for the channel, columns, pumps and valves. There may be a manger of microfluidics that initializes, directs, adjusts flow motion based on electrokinetic flow force and the concepts of channel geometry, fluid flow rates, and diffusion coefficients. Flow control can be neural network-based through use of embedded software that manages microfluidic circulation, switches valves, and activates pumps based on predefined order and timeframe. The column of reaction may a wall that is an artificial membrane made from self-assembling. The column of reaction presents bioaffinity-based “hooks” which are immobilized antibodies. The strain-specific molecule on the membrane of the distinctive E. coli cell binds to the antibodies and thus the cell is attached with the wall until elution. The Vertical Cavity Surface Emitting Laser (VCSEL) 524 originates laser beams through the tiny cavities and thus permits output light to be analyzed in a spectrometer to detect changes after the E. coli cell binds to the affinity hooks.

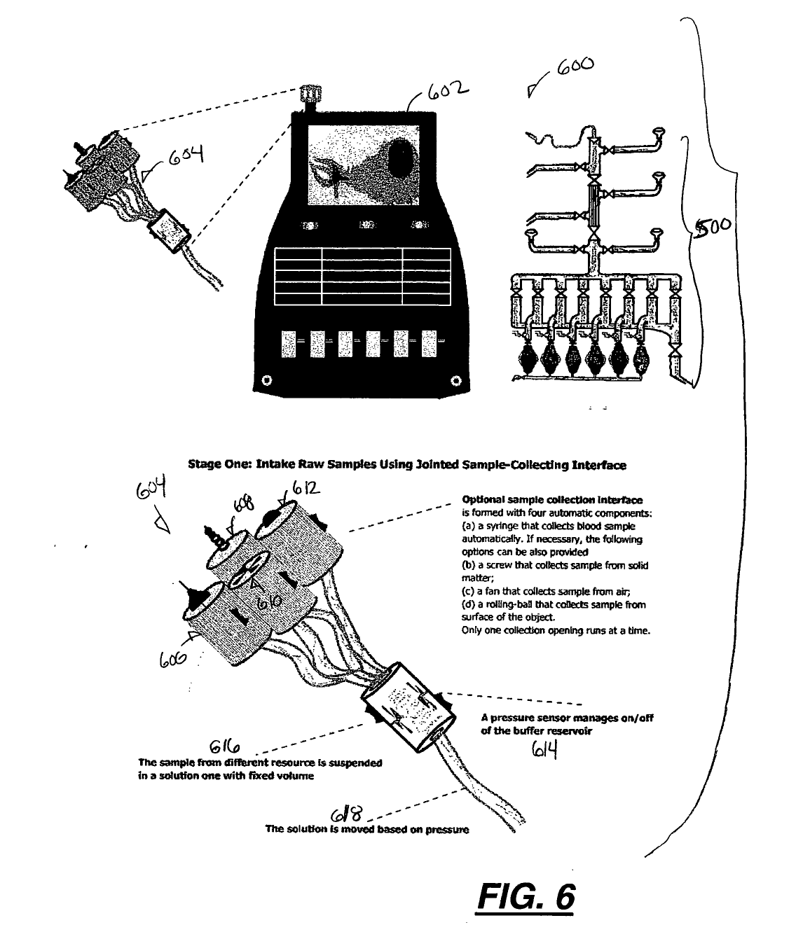

FIG. 6 illustrates an E. coli detector according to one embodiment of the present invention. The E. coli detector includes a housing 602 with a sample-collecting interface 604 extending from the housing for collection of samples. The sample-collecting interface 604 is preferably formed from four automatic components. These include a syringe 606 that collects blood samples automatically, a screw 608 that collects samples from solid matter, a fan 610 that collects samples from air, and a rolling ball 612 that collects samples from the surface of an object. Only one collection opening runs at a time in the embodiment shown. The sample from different resources is suspended in a solution with a fixed volume within a vessel 616. A pressure sensor is used to manage on/off state of a buffer reservoir. The solution is moved based on pressure through the tube 618.

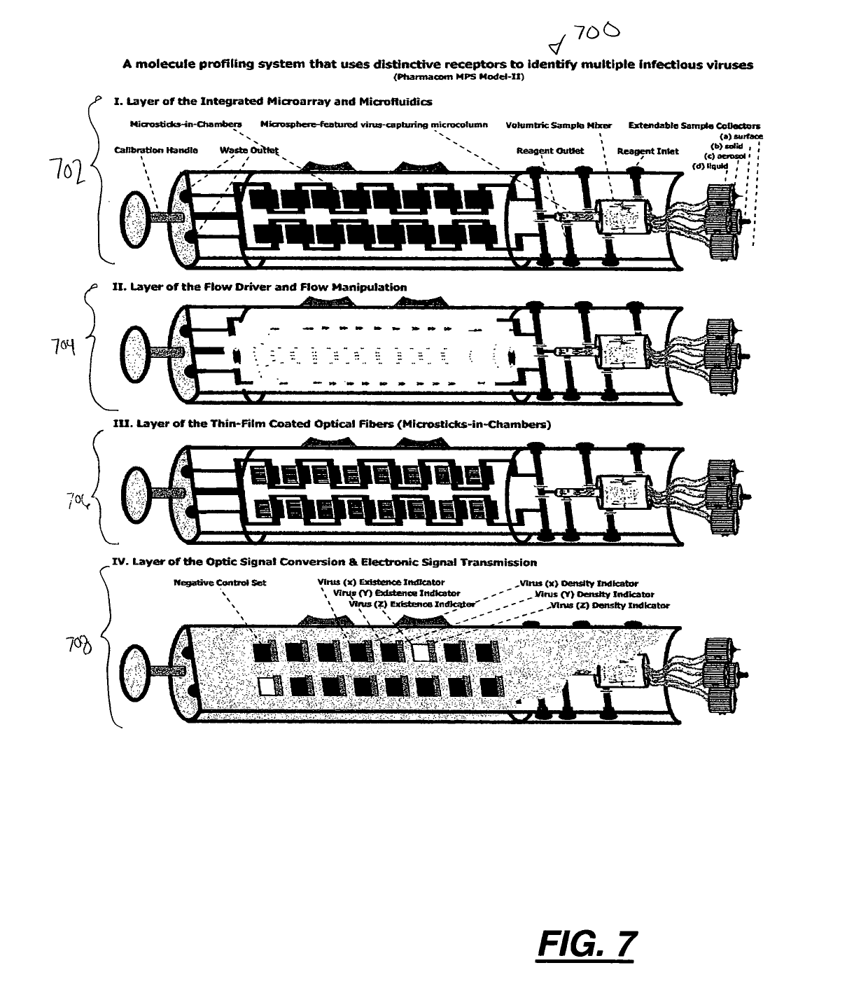

FIG. 7 illustrates one embodiment of a virus identification microsystem of the present invention. The microsystem 700 is a molecule profiling system that uses distinctive receptors to identify multiple infectious viruses. The microsystem 700 can be used to identify TB signature molecules; human and animal forms of SARS virus; Influenza Type A, B or C; or prion proteins of BSE through sampling from blood, urine, saliva or body fluids. The microsystem 700 is shown in four separate layers. There is a layer of integrated microarray and microfluidics 702, a layer of the flow driver and flow manipulation 704, a layer of the thin-film coated optical fibers (microsticks-in-chambers) 706, and a layer of the optic signal conversion and electronic signal transmission 708.

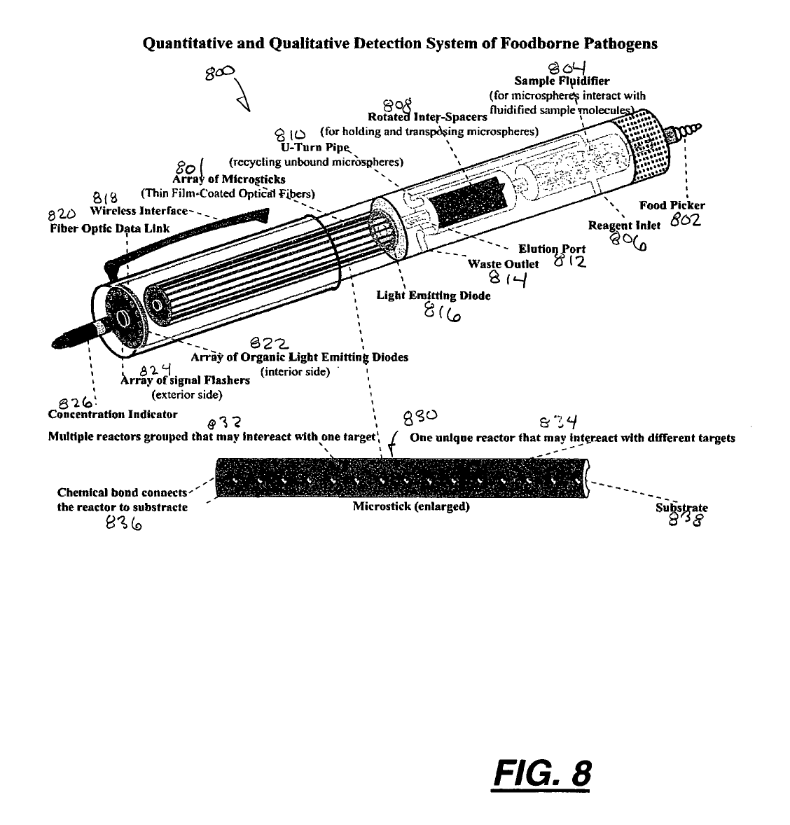

FIG. 8 illustrates one embodiment of a food inspection microsystem according to the present invention. The microsystem 800 is a multiple sampling interface microsystem that is designed to identify bacteria and bacterium substrains during meat processing and packaging through a combinatorial detection of selected surface molecules. Note that the system 800 uses an array 801 of microsticks (thin-coated optical fibers). In the embodiment shown, a food picker 802 is used to sample food. A sample fluidifer 804 is shown for fluidizing the sample. There is a reagent inlet 806 which can be used to add reagent to the sample fluidifier as needed. Microspheres interact with the sample within the sample fluidifier. There are rotated inter-spacers 808 for holding and transporting the microspheres and releasing the microspheres into the sample fluidifier. A U-turn pipe 810 is shown for recycling unbound microspheres from the sample fluidifier 804. An elution port 812 is shown. Also a waste outlet 814 is provided. A light emitting diode 816 is shown which is directed into the array of microsticks 802. At the opposite end of the array of microsticks 802 is an array 822 of organic light emitting diodes (interior side). An array of signal flashers 824 is shown on an exterior side of a housing. A concentration indicator 826 is shown. A fiber optic data link 820 and a wireless interface 818 are also provided. The microsystem of FIG. 8 provides for quantitative and qualitative detection of foodborne pathogens.

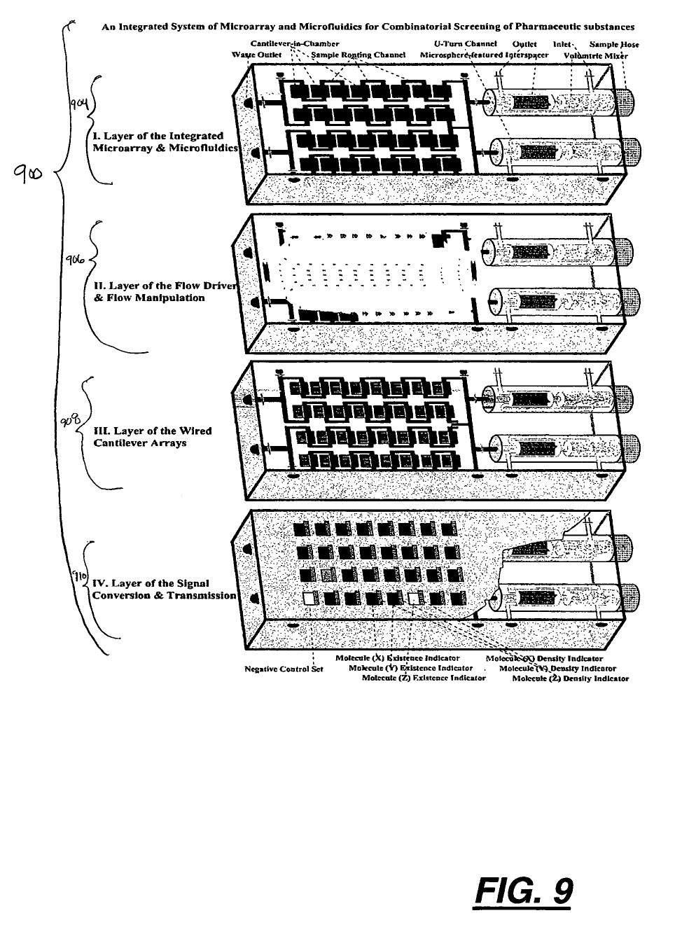

FIG. 9 illustrates one embodiment of a pharmaceutical substance screening microsystem according to one embodiment of the present invention. The microsystem 900 is designed to perform high throughout screening of bacteria which are drug-sensitive. The microsystem 900 includes a first layer of integrated microarray and microfluidics 904, a second layer of the flow driver and flow manipulation 906, a third layer of the wired cantilever arrays 908, and a fourth layer of the signal conversion and transmission 910.

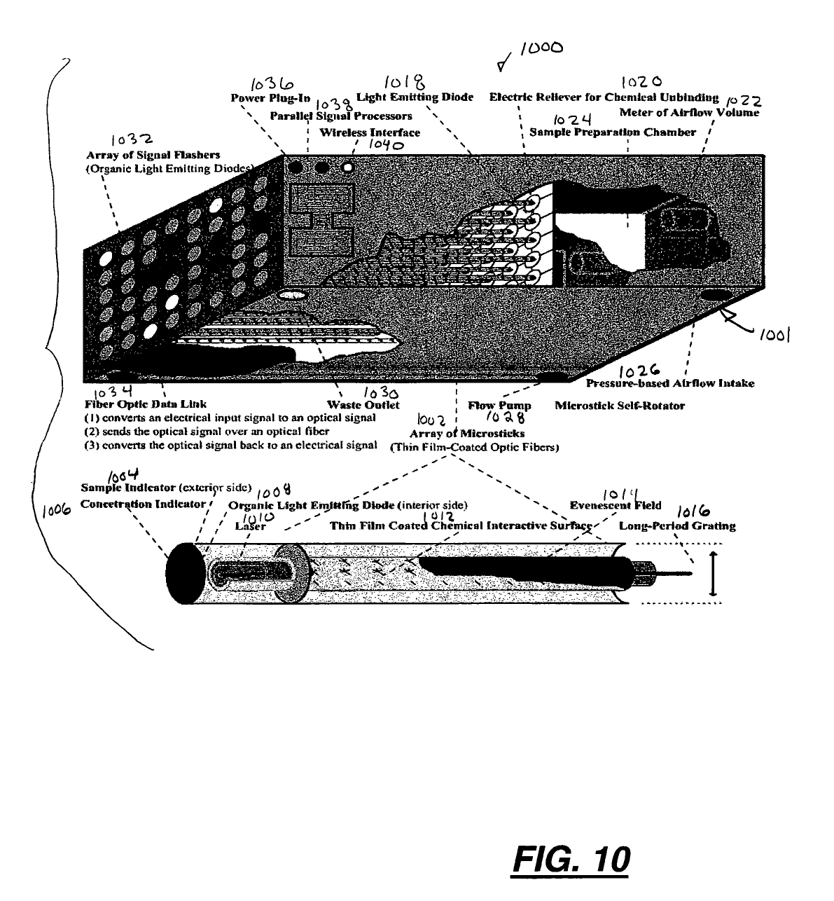

FIG. 10 illustrates an embodiment of an aerosol monitoring microsystem (interior use embodiment). The microsystems shown is designed to monitor environmental agents based on sampling from air, liquid and solid materials. This is a biological confirmation and detection system that functions stand-alone or/and operates by coupling with an air concentrator. It is able to undertake all steps in a streamlined analysis from raw sample collection, optional sample separation, captured target identification, signal transmission and remote reporting. It is designed to detect bioaerosol agents at government buildings, airports, subways, office buildings, shopping malls, sports arenas, hotels, and hospitals based on both point and volumetric sensing mechanisms. It is designed to have the capacity of (1) performing a point sensing by auto-taking a small volume of aerosol samples from its ambience (collecting at 50 different points within a range of 0.1 to 10 meters) if it is stand-alone as well as (2) performing a volumetric sensing when it is coupled with the air concentrator, a configured portable module that can process a large volume (1,000 liters of air per minute) of aerosol samples. As shown in FIG. 10, within a housing 1001 an array of microsticks 1002 are used. It is to be understood that a microstick preferably includes thin-film optic fibers. In the configuration shown, on an exterior side of each microstick is a sample indicator 1004. There is also a concentration indicator 1006 present. On an interior side is an organic light emitting diode 1008. A laser 1010 is shown. A thin film coated chemical interactive surface 1012 is shown. An evanescent field 1014 is shown as well as a long-period grating 1016. For each microstick within the array there is a light emitting diode 1018 and an electric reliever for chemical unbinding 1020. In operation a sample from the environment is taken through a pressure-based airflow intake 1026 and through the meter of airflow volume 1022 and into the sample preparation chamber 1024. A flow pump 1028 is shown to assist in airflow. A waste outlet 1030 is also provided. The air samples are processed by the array of microsticks 1002. On the opposite side of the array of microsticks 1002 is an array of signal flashers 1032. A fiber optic data link 1034 is shown. A power plug-in 1036 is provided. Parallel signal processors 1030 are used within the system. A wireless interface 1040 is also provided.

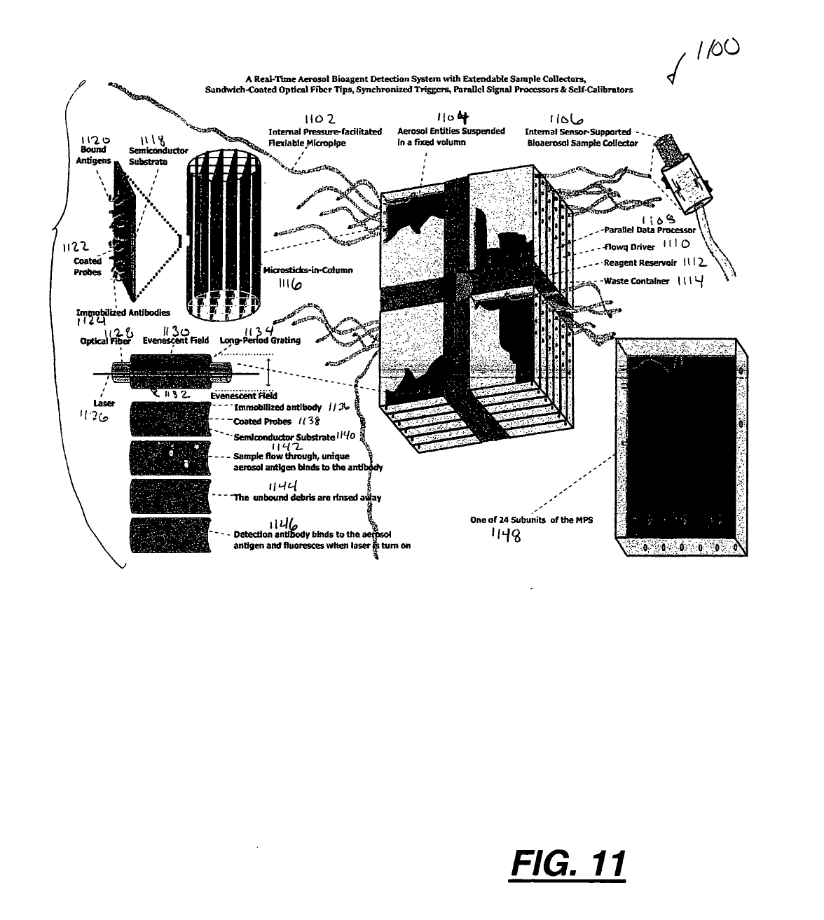

FIG. 11 illustrates one embodiment of an aerosol monitoring microsystem for exterior use. This is a real-time bioaerosol detection system with extendable sample collectors & parallel sample processors. It is designed to work as short-range on-demand low volatility chemical detector, stand-off remote-controlled biothreat monitor, or environmental toxin detector. The system 1100 shown includes an internal pressure-facilitated flexible micropipe 1102 with an internal sensor-supported bioaerosol sample collector 1106 on the end of the micropipe 1102. Incoming aerosol entities are suspended in a fixed volume 1104. Microsticks-in-column 1116 are shown which include a semiconductor substrate 1118 with coated probes 1122, immobilized antibodies 1124, bound antigens 1120, and immobilized antibodies 1124.

A laser beam 1126 is shown directed through an optical fiber 1128 which has a thin film coating 1132. An evanescent field 1130 is shown along with long-period grating 1134. A semiconductor substrate 1140 is shown with coated probes 1138, and an immobilized antibody 1136. As the sample flows through a unique aerosol antigen 1142 binds to the antibody as shown. The unbound debris 1144 are rinsed away. The detection antibody binds to the aerosol antigen and fluoresces when the laser is turned on.

The system 1100 also includes a parallel data processor 1108, a flow driver 1110, a reagent reservoir 1112, and a waste container 1114. The system preferably comprises a plurality of subunits 1148.

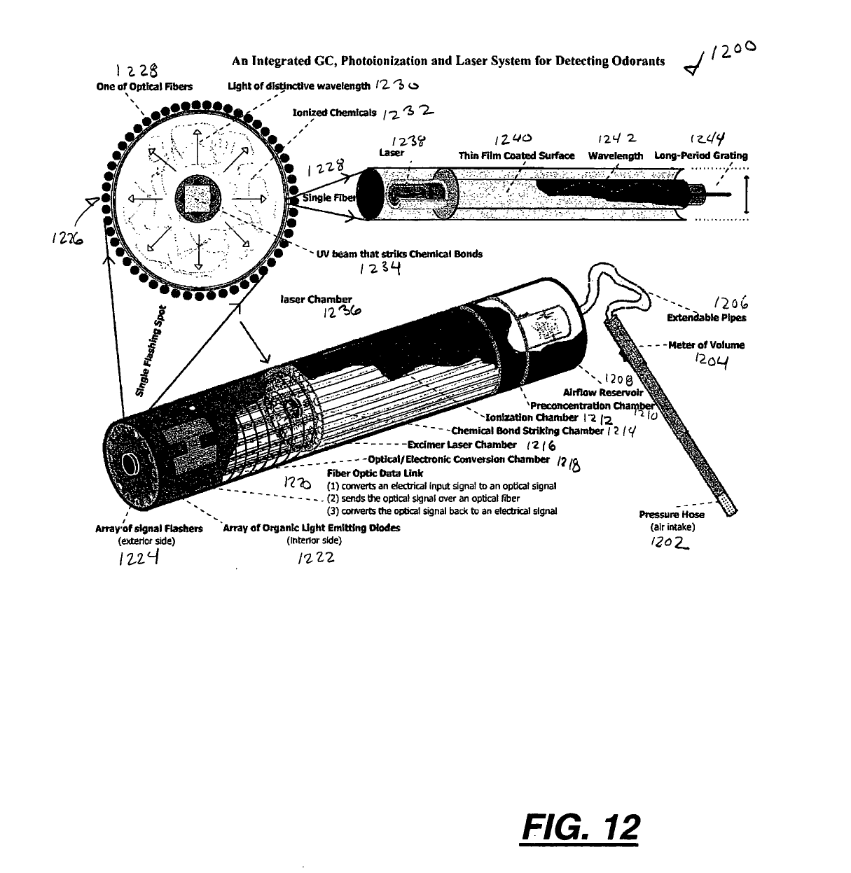

FIG. 12 illustrates one embodiment of a system that provides for simultaneously detecting and distinguishing multiple odorant molecules. The handheld molecule profiler shown is adapted to specifically detects heroin, GHB and GBL, or other nerve agents. The system 1200 includes a pressure hose 1202 that provides for air intake. There is a meter of volume 1204. Extendable pipes 1206 connect with the main body and into an airflow reservoir 1208. A preconcentration chamber 1210 is positioned between the airflow reservoir 1208 and an ionization chamber 1212. At an opposite end of the ionization chamber 1212 is a chemical bond striking chamber 1214. An excimer laser chamber 1216 is present. An optical/electronic conversion chamber 1218 is also present. A fiber optic data link 1220 is also shown. The fiber optic data link 1220 can be used to convert an electrical input signal to an optical signal, send the optical signal over an optical fiber and then convert the optical signal back to an electrical signal at the other end of the optical fiber. An array of light emitting diodes 1222 are also shown on an interior side of the main body with an array of signal flashers 1224 disposed on an exterior side of the main body. The array of light emitting diodes 1224 includes a plurality of flashing spots 1226. Each of the flashing spots 1226 includes a number of optical fibers 1228. As shown in FIG. 12, a UV beam 1234 strikes chemical bonds. Ionized chemicals 1232 are shown. Light of distinctive wavelength 1230 is shown. A single fiber 1228 is shown with an associated laser 1238, thin film coated surface 1240, wavelength 1242, and long-period grating 1244.

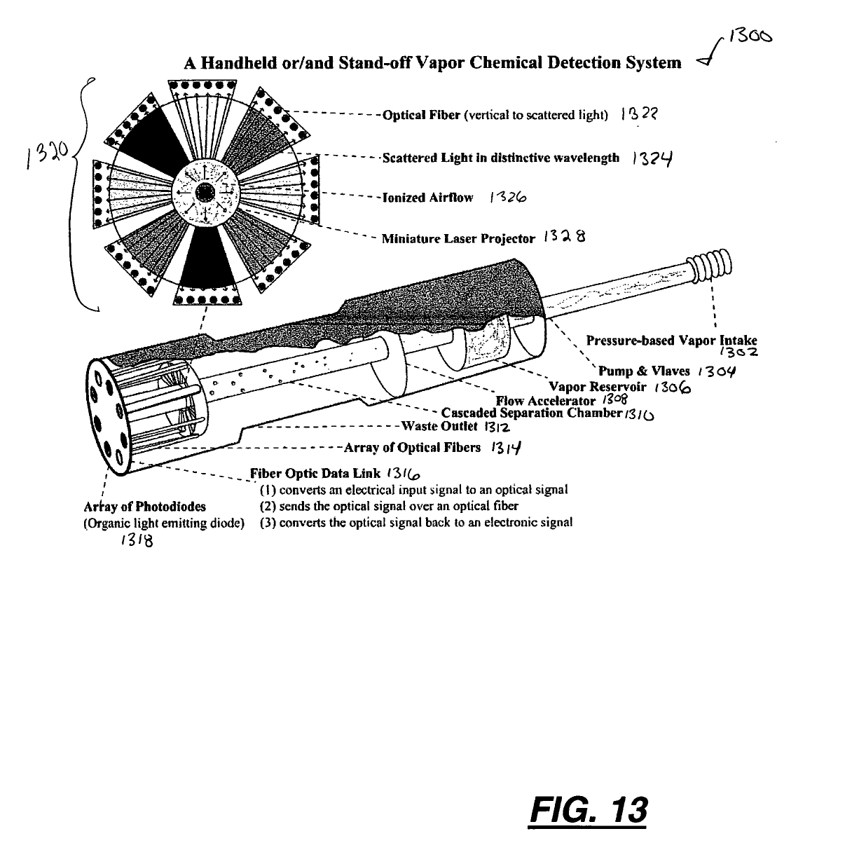

FIG. 13 illustrates one embodiment of a microsystems designed to work as a short-range on-demand low volatility chemical detector, a stand-off remote-controlled biothreat monitor, or an environmental toxin detector. The detection system 1300 includes a pressure-based vapor intake 1302. Pump and valves 1304 are shown. Vapor is thus pumped into a vapor reservoir 1306. A flow accelerator 1308 is provided. A cascaded separation chamber 1310 is then used. A waste outlet 1312 is provided. An array of optical fibers 1314 is provided. An array of photodiodes 1318 (organic light emitting diodes) are shown. A subsystem 1320 is shown with an optical fiber 1322, scattered light in distinctive wavelength 1324, ionized airflow 1326, and a miniature laser projector 1328.

The handheld molecule profiler system 1300 specifically detects heroin, GHB and GBL, or others nerve agents. The performance of the system involves several key steps and critical components. (1) Extendable Sample Collection Interface & Flow Processing: airflow is auto-collected through a pressure-based filter and goes to an enrichment process with supporting agents. (2) Photoionization: following the enrichment the sample is introduced into the photoionization chamber where the compounds are further selected using an ionization method that preferentially ionizes the compounds of interest. Explosives have properties that make them very reactive to negative ions and electrons. The selectively ionized molecules are then detected by a method that further differentiates different compounds, after ionization samples are routed into separated chambers that are arranged in parallel. (3) Laser Exciting & Chemical Bond Breakage: Each individual chamber is supported with a laser-mediated “chemical bond striker” that only breaks one type of chemical bond at once in a fixed interior environment. Following the strike emissions with a distinctive wavelength is generated in a certain density. (4) Microsticks & Fiber Optic Data Link: an array of optical fibers which is vertically arranged to the energy-generating chamber transduce the unique wavelength to the organic light emitting diodes for signaling. Three actions can be optionally executed at this stage: (a) convert an electrical input signal to an optical signal; (b) send the optical signal over an optical fiber; and/or (c) convert the optical signal back to an electrical signal. An electronic wave that was initially generated by a type of microstick can be utilized “locally”, which is to excite a type of organic light emitting diode that flashes signals. It can be also used remotely, that is to be treated as a data-carrier for signal transmission. (5) Parallel Signal Processing: Neural network algorithm-based combinatorial code board and the artificial intelligence system are combined as core parts of the parallel signal processor, which allows orchestrating data flow generated from hundreds of samples and their signaling channels simultaneously. (6) Signal Transmission: the UWB receiver is disposed remote from and within range of the transmitter receives and converts the UWB signal to a signal containing information from the transmitter reading.

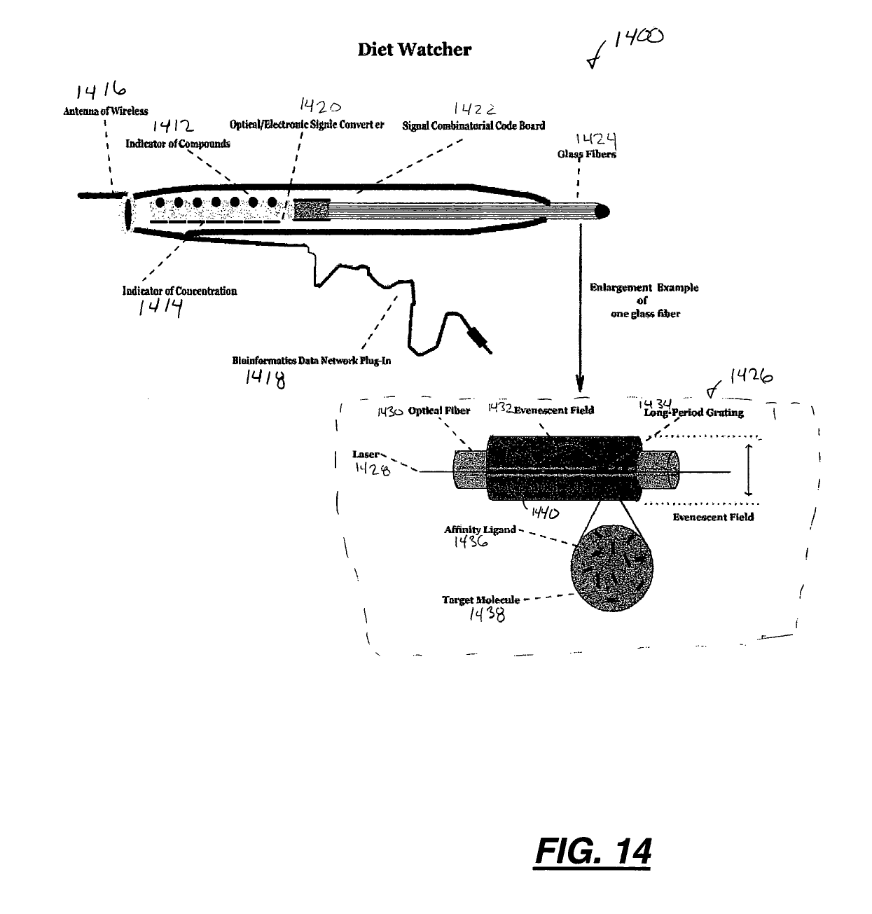

FIG. 14 illustrates one embodiment of a diet watcher of the present invention. The rapid and portable monitor of FIG. 14 can be used for determining vitamin A status or measure caloric level and diet figure. As shown in FIG. 14, signaling interface 1402 is shown. Immobilized beads 1404 are shown. There is an inlet for reaction reagents 1406 and an inlet of signal amplification reagents 1408. There is a fluorescent/electronic signal converter 1410 an indicator of compounds 1412 and an indicator of concentration 1414. An antenna 1416 is provided for wireless communications. A bioinformatics data network plug 1418 is also provided. An optical/electronic signal converter 1420 is shown as well as a signal combinatorial code board 1422. A plurality of glass fibers 1424 are also shown at the tip of the device. An enlarged view 1426 of a single glass fiber in operation is also provided. A laser beam 1428 is directed through an optical fiber 1430. A long period grating 1434 and an evanescent field 1432 are shown. There is a polymer film coating 1440. An affinity ligand 1436 and target molecule 1438 are shown on the coating.

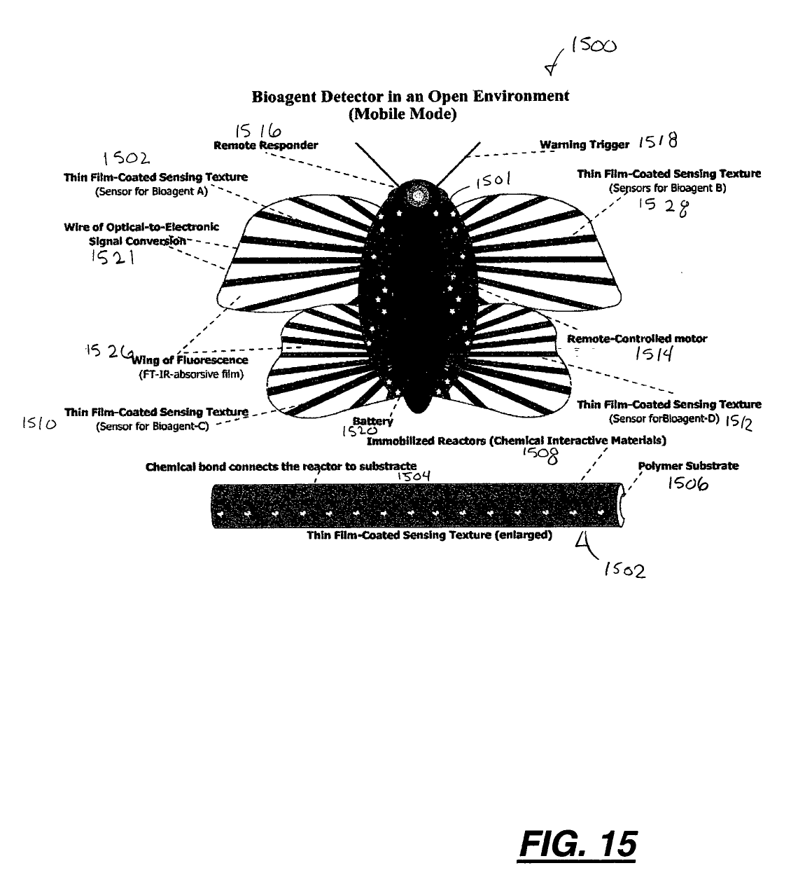

FIG. 15 illustrates one embodiment of a bioagent detector for use in an open environment which is sized and shaped to resemble a butterfly. The butterfly is a stand-alone sensor designed to detect explosives at a safe distance and prevent the operator from blast effects. It can provide surveillance and detection at distances greater than 10 meters at the shells of vehicles, suitcases, packages, clothing, or any unreachable area and individual suspects. The butterfly is the electronic version of the dog olfactory system. It is a grouped circuits that functions similarly to the proteins in the epithelium. In this system, multiple arrays of compound-binding polymer textures are placed in the wings along conductive pathways. Each texture is sensitive to a specific compound and will respond to its presence. This binding in turn alters the electrical conductivity of the pathway along which the polymer texture rests. A measure of changes in resistance with exposure to a vapor in several dozen of such polymers results in a pattern of responses. This pattern can then be matched to a specific compound and identified. The increased numbers of the polymer texture enhances the discrimination and the reflection of fluorescence can be triggered when the numbers of the binding instance occurred at the polymer textures reaches a predefined level.

The system 1500 has a body 1501 preferably sized and shaped to resemble an insect or other life form associated with the open environment in which sensing occurs. The system 1500 has a thin film-coated sensing texture 1502 which acts as a sensor for a first bioagent. An example of a thin film-coated sensing texture 1502 is shown in an enlarged view. Note that a chemical bond 1504 connects the reactor to a substrate. A polymer substrate 1506 is shown with immobilized reactors 1508 (chemical interactive materials). A second thin film-coated sensing texture 1528 which acts as a sensor for a second bioagent. A third thin film-coated sensing texture 1510 acts as a sensor for a third bioagent. Other components of the system include a remote responder 1516, a warning trigger 1518, a remote-controlled motor 1514, a battery 1520, and a wing of fluorescence 1526 (such as an FT-IR-absorptive film). The embodiment of FIG. 15 can be altered to resemble alternative life forms, include different numbers of sensors, sensors for varying types of bioagents, and in other ways as a particular use may require.

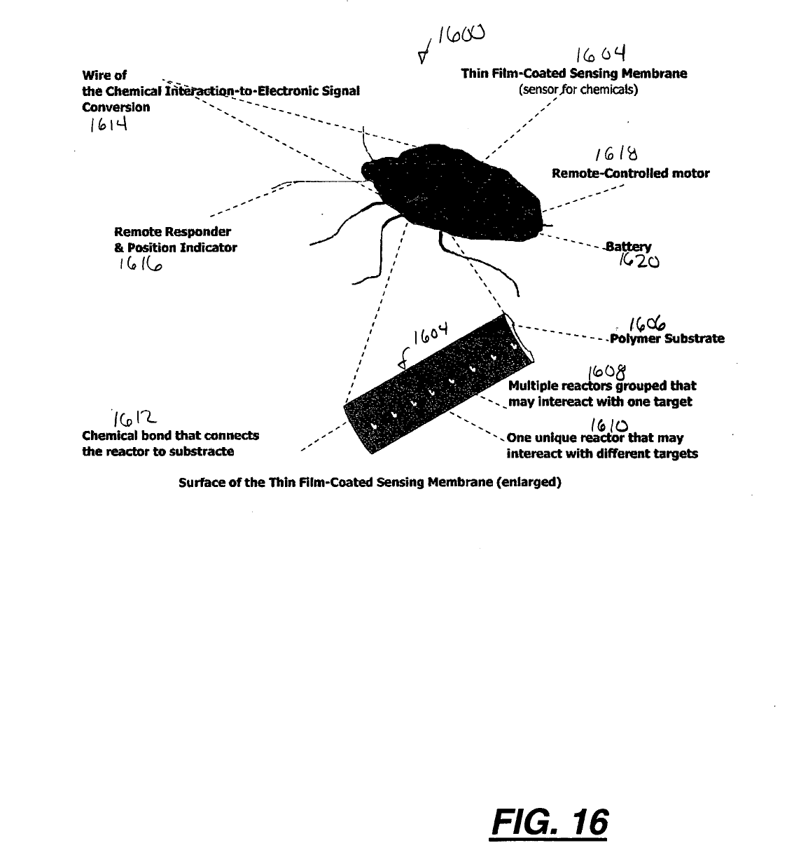

FIG. 16 discloses another embodiment of the present invention. It is one stand-alone mobile subunit of the networked mobile microsystem that is used to detect human scents at unreachable areas. A moving bug is the electronic version of the dog olfactory system. The thin film-coated (chemical interactive material) membrane function similarly to neurotransmitters in the epithelium and the grouped circuits works like the fibers in the nerve system. In the device, multiple arrays of compound-binding sensing membrane are placed in the wings along conductive pathways. Each texture on the wing is sensitive to the specific chemicals of human smell and will respond to their presence. The binding in turn alters the electrical conductivity of the pathway along which the sensing texture rests. A measure of changes in resistance with exposure to human odorants due to certain numbers of chemical reactions on the membrane results in a pattern of responses. This pattern can then be matched to a specific compound and identified. The increased number of the binding enhances the discrimination and the electronic signals can be triggered when the numbers of the binding instance occurred on the sensing membrane reaches a predefined level. A system 1600 is shown. It is one stand-alone mobile subunit of the networked mobile microsystem that is used to detect human scents at unreachable areas. The system has a body 1602 sized, shaped, and/or colored to resemble an insect. Of course, the present invention contemplates the body 1602 can be otherwise sized, shaped, and/or colored as may be appropriate in a particular application or environment. The system 1600 includes one or more thin film-coated sensing membrane 1604 disposed on the body 1602. An enlarged view of a thin film-coated sensing membrane 1604. Note that there is a polymer substrate 1606 with multiple reactors 1608 which are grouped and may interact with one target. Or there may be one unique reactor 1610 that may interact with different targets. A chemical bond 1612 is shown that connects the reactor to the substrate. The system 1600 also includes such components as a wire of the chemical interaction-to-electronic signal conversion 1614, a remote responder and position indicator 1616, a remote-controlled motor 1618, and a battery 1620.

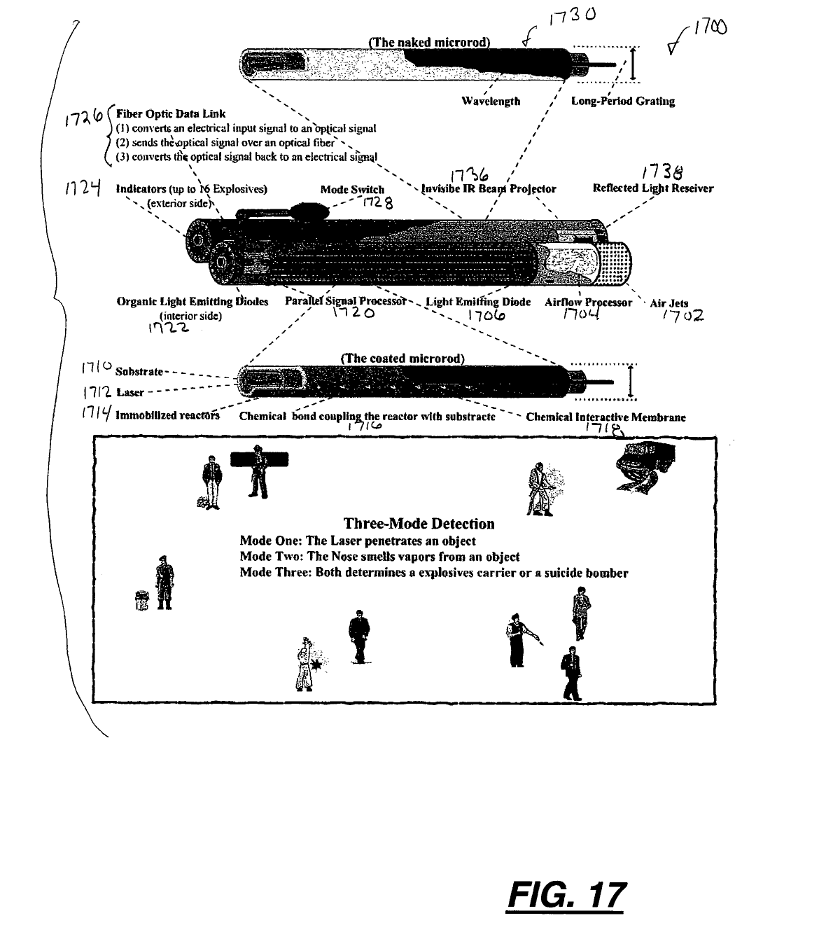

FIG. 17 illustrates one embodiment of a forensics microdetection system 1700. The system shown is a handheld system that is able to sample trace level materials from blood, urine, saliva, body fluids or the surface of objects. The system 1700 includes a plurality of air jets 1702. There is an airflow processor 1704. A light emitting diode 1706 is shown. A coated microrod 1708 is illustrated which includes a substrate 1710, a laser 1712, immobilized reactors 1714, a chemical bond 1716 coupling the reactor with substrate 1710, and a chemical interactive membrane 1718. The system 1700 also includes a parallel signal processor 1720, a plurality of organic emitting diodes 1722 on the interior of the system 1700 and indicators 1724 (shown for 16 different types explosives) on the exterior of the system 1700. A fiber optic data link 1726 is also shown, as well as a mode switch 1728, invisible IR beam projector 1736, and reflected light receiver 1738. A naked (uncoated) microrod 1730 is also shown with an associated wavelength 1732 and having a long-period grating 1734.

The mode switch 1728 allows the mode to be selected. In a first mode, the laser penetrates an object. In a second mode, the nose smells vapor from an object. In a third mode, both the laser and the nose are used together to determine an explosive carrier or a suicide bomber.

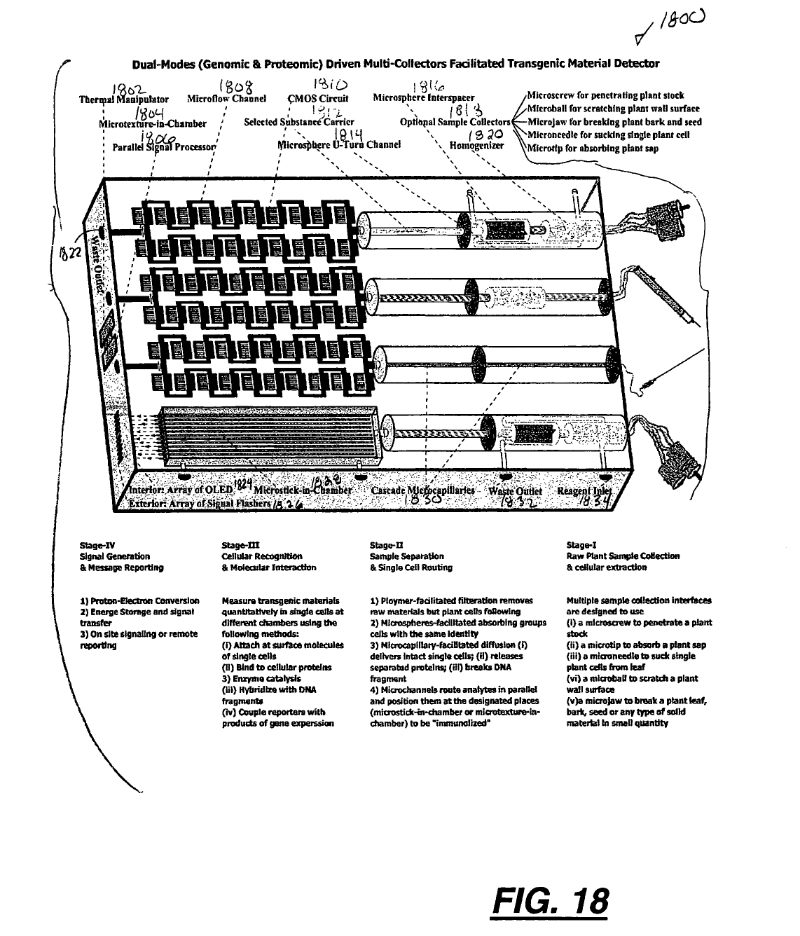

FIG. 18 illustrates a dual mode system 1800 that identifies trace level transgenic materials through both genomic and proteomic simultaneous testing. The testing occurs in a four stage process. In the first stage, raw plant sample collection and cellular extraction occurs. Multiple sample collection interfaces can be used including a microscrew to penetrate a plant stock, a microtip to absorb a plant sap, a microneedle to suck single plant cells from leaf, a microball to scratch a plant wall surface, a microjaw to break a plant leaf, bark, see or any type of solid material into a small quantity. In a second stage sample separation and single cell routing occurs. Polymer-facilitated facilitation removes raw materials except for the plant cells. Micro-sphere-facilitated absorbing groups cells with the same identity. Microcapilliary-facilitated diffusion delivers intact singles cells, releases separated proteins and breaks DNA fragments. Microchannels route analytes in parallel and position them at the designated places (microstick-in-chamber or microtexture-in-chamber) to be immunolized. In the third stage cellular recognition and molecular interaction occurs. The transgenic materials are measured quantitatively in single cells at different chambers using methods such as attaching at surface molecules of single cells, binding to cellular proteins, enzyme catalysis, hybridize with DNA fragments, or couple reporters with products of gene expression.

In the fourth stage, signal generation and message reporting occur. This can include proton-electron conversion, energy storage and signal transfer, and on-site signaling or remote reporting.

As shown in FIG. 18, optional sample collectors 1818 can include a microscrew for penetrating plant stock, a microball for scratching a plant wall surface, a microjaw for breaking plant bark and seed, a microneedle for sucking single plant cells, or a microtip for absorbing plant sap. A homogenizer 1820 is also shown, as well as a microsphere interspacer 1816, a microsphere U-turn channel 1814, a selected substance carrier 1812, a CMOS circuit 1810, a microflow channel 1808, a parallel signal processor 1806, a microtexture-in-chamber 1804, and a thermal manipulator 1802. A waste outlet 1822 is also provided. An interior array of OLED 1824 is shown as well as an exterior array of signal flashers 1826. Microstick-in-chamber 1828 are shown as well as cascaded microcapillaries 1830, a waste outlet 1832, and a reagent inlet 1834.

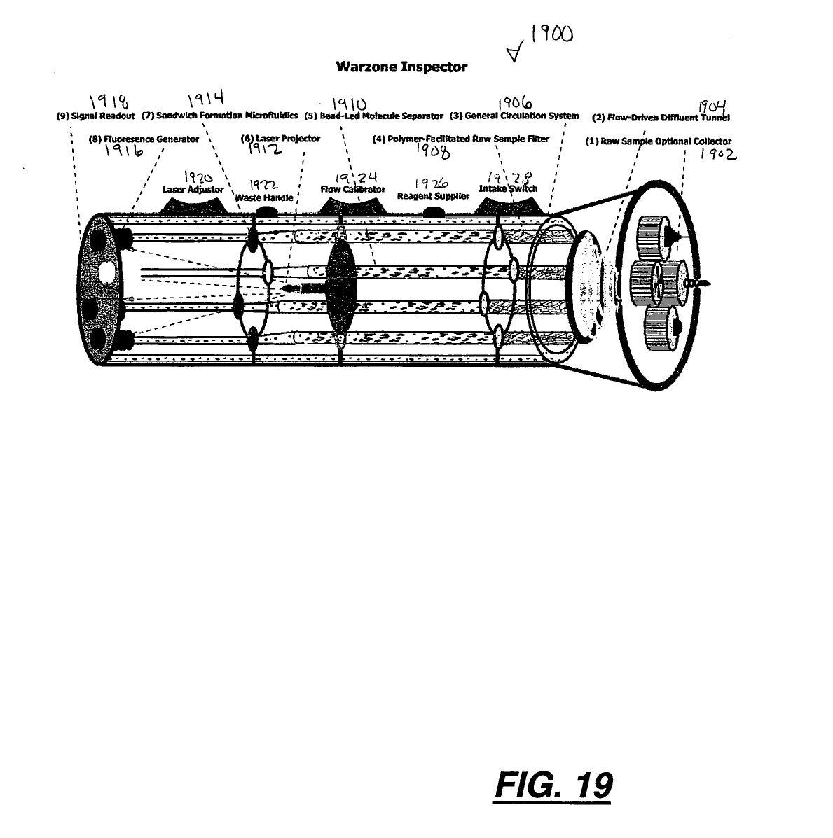

FIG. 19 illustrates one embodiment of a warzone inspection system 1900 of the present invention. The system shown is a lightweight system that identifies trace level biothreat agents at warzone and suspicious areas. The warzone inspection system 1900 includes four separate stages. The first stage is the raw sample gathering and intaking stage 1910. The second stage is the cell separating and single cell transferring stage 1908. The third stage is the molecular immuno interaction stage 1906. The fourth stage is the laser exciting and signaling stage 1904. In the raw sample gathering and intake stage 1910, the sample collection interface has a mechanism of automation which is made using a needle that collects a sample from a liquid, a mini-screw that collects samples from solid items, a mini fan that collects samples from air, and a rolling-ball that collects samples from a surface of an object. In the cell separating and single cell transferring stage 1908 the method performed includes differentiating the hopeful cells, eliminating all non-specific cells and entities, grouping a single cell, routing the separated single cell to its next destination, positioning the single cell at the place to be immunolized, and arranging a range of single cells in parallel at the place to be examined. In the molecular immuno interaction stage 1906, the method performs includes targeting surface molecules of the single cells and generating triplex of anti-gen-antibody-target molecule. In the laser exciting and signaling stage 1904, the method performed includes, exciting the targeted molecule, emitting multi-fluorescence light corresponding to a class of single cell, and visualizing the specific indications.

The warzone inspection system 1900 shown has a body 1902. At one end of the body 1902 is a raw sample optional collector 1912. Next is a microfluidic diffluent channel 1914. A bead-facilitated cell separator 1916 is next. An antibody-guided single cell separator 1918 is also shown. Next is an immuno sandwich maker 1920 and dual interfaces 1922. A laser beam 1924 is next followed by a laser projector 1926. A readout 1928 is disposed on the outside of an end of the body 1902 opposite of the raw sample optional collector 1912. Also shown are a flow gate 1930, liquid calibration cycler 1932, and a light flapper 1934.

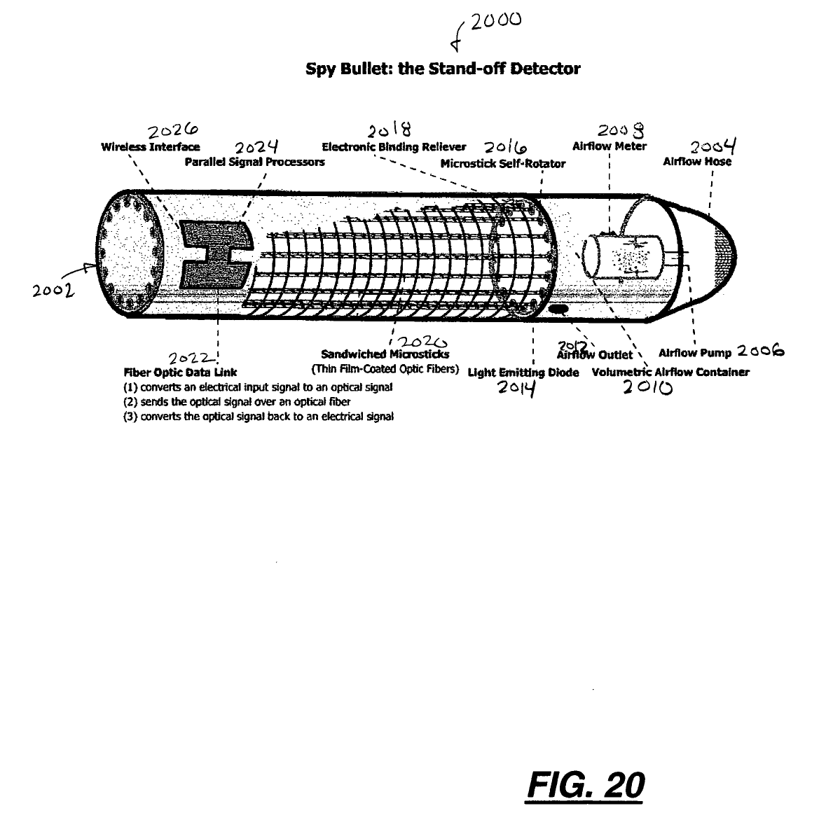

FIG. 20 illustrates one embodiment of an unattended monitoring microsystem according to one embodiment of the present invention. That which is shown is a fully automatic system that frequently or non-stop monitors biothreat agents or environmental factors at any designated area. A stand-off detector microsystem 2000 sized and shaped like a bullet is shown. The system 2000 includes a body 2002 sized and shaped like a bullet. There is an airflow hose 2004, an airflow pump 2006, an airflow meter 2008, a volumetric airflow container 2010, an airflow outlet 2012, a light-emitting diode 2014, a microstick self-rotator 2016, an electronic binding reliever 2018, a plurality of sandwiched microsticks 2020, a fiber-optic data link 2022, parallel signal processors 2024, and a wireless interface 2026.

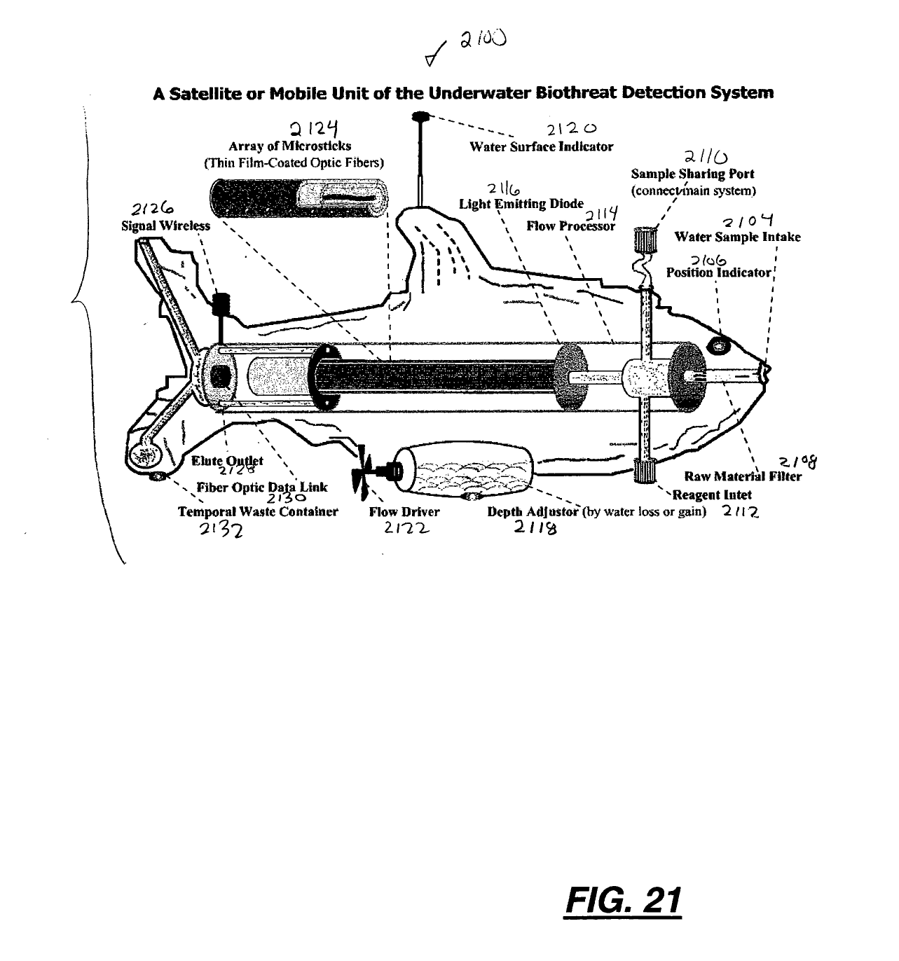

FIG. 21 illustrates one embodiment of an underwater surveillance microsystem 2100. The microsystem 2100 is a wireless-featured mobile system that swims in water for constant detection of bioagents. The underwater surveillance microsystem 2100 includes a fish body 2102 designed to resemble a fish such as by having a size, shape, or color associated with a fish. A water sample intake 2104 is positioned at the mouth. A position indicator 2106 is positioned at an eye. Disposed within the body 2102 is a raw material filter 2108. There is a sample sharing port 2110 for connection to a main system and a reagent inlet 2112. A flow processor 2104, light emitting diode 2116 are also within the body 2102. A depth adjustor 2118 allows water loss or gain to be adjusted to thereby alter the depth of the device. A flow driver 2122 is also provided. A water surface indicator 2120 is positioned on the outside of the device. An array of microsticks 2124 (such as thin-film coated optic fibers) are positioned within the device. A wireless signal 2126, elute outlet 2128, fiber optic data link 2130, and temporal waste container 2132 are also provided.

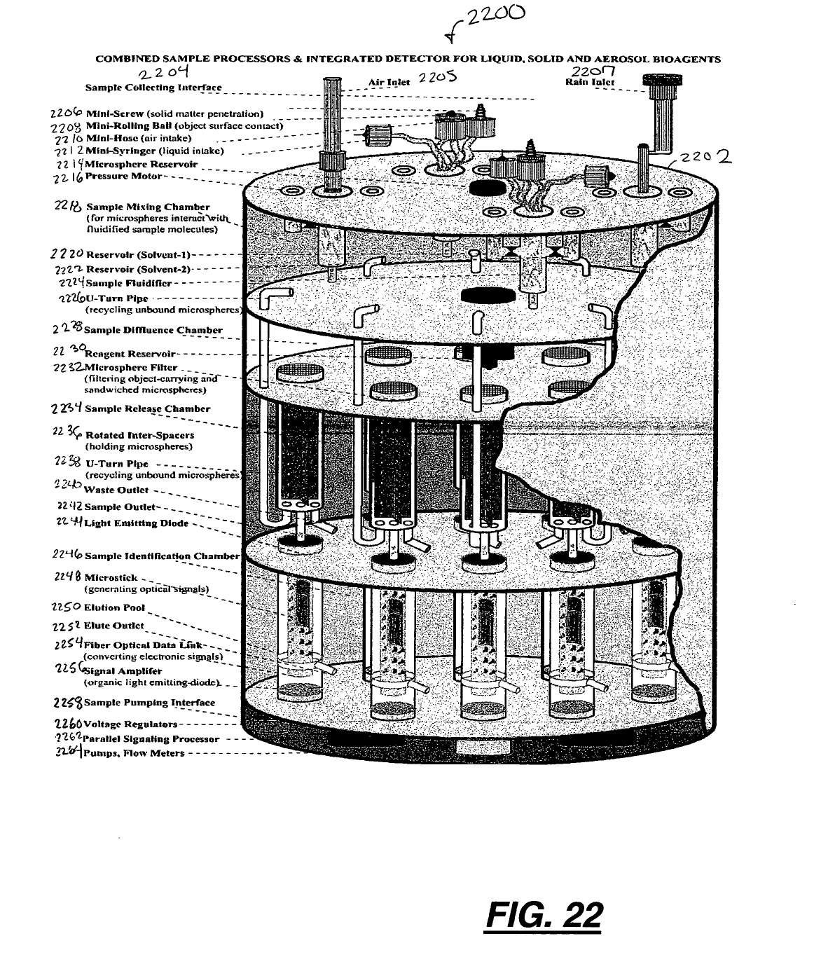

FIG. 22 illustrates one embodiment of an air surveillance microsystem 2200. It is an automatic system with combined sample processors and integrated detectors for liquid, solid and aerosol bioagents at an open environment. The first level is the sample mixing chamber 2218. It holds raw samples and mixes them with a variety of microsphere populations. Each type of microspheres is designed to bind unique objects of interest, include chemical and biological agent respectively based on each of individually fabricated molecule at the surface of the microspheres. The second level is the sample diffluence chamber 2228. Different sized filters 2232 are arranged as the opening and the correspondent microcolumns are joined at the bottom. Each filter permits only one type of microspheres to flow through. The third level is the sample release chamber 2234. Inter-spacers 2236 inside the microcolumn are made of electrostatic membranes. The membrane interacts with and binds to entered microspheres based on an electrostatic mechanism. The object which is carried by a microsphere is stripped away from the microsphere through the first elution, and the microsphere itself remains attachment with the membrane. The naked microsphere will be held by the membrane temporally until the second elution. Three types of micropipes are implemented is connected with a microcolumn at the bottom. The first pipe 2242 leads eluted samples from the container into analytical chambers, the second pipe 2240 delivers waste to an outlet after elution, and the third pipe 2238 pumps stripped microspheres back to the up-level for next run. The fourth level is the sample identification chamber 2246. It is the place where a sandwich reaction occurs between a living object bearing unique surface molecule, a biomolecule itself or a chemical agent (which are eluted from the microcolumn at the level three) and a thin film-coated microstick. Each microchamber contains an array of microsticks 2248 (numbers from 10 to 100) that can specifically recognize one unique living object or unique molecule. The microstick 2248 generates a proper optical signal when an event of molecule recognition. The optical signal will be converted to be an electronic signal through the fiber optical data link 2254. An array of microsticks is correspondent to an array of signal flashers in parallel.

As shown in FIG. 22, the air surveillance microsystem 2200 has a housing 2202. There is a sample collecting interface 2204 shown on a top portion of the housing 2202. The sample collecting interface 2204 includes an air inlet 2205, and a rain inlet 2207. A mini-screw 2206 is shown to provide for sampling via solid matter penetration. A mini-rolling ball 2208 is shown for object surface contact sampling. A mini-hose 2210 is shown for air intake. A mini-syringe 2212 provides for liquid intake. A microsphere reservoir 2214 is also provided. A pressure motor 2216 is also shown.

The sample mixing chamber 2218 allows microspheres to interact with fluidized sample molecules. There is a reservoir 2220 for a first solvent, a reservoir 2222 for a second solvent, a sample fluidifer 2224, and a u-turn pipe 2226 for recycling unbound microspheres.

The sample diffluence chamber 2228 includes a reagent reservoir 2230, and a microsphere filter 2232 that provides for filtering object-carrying and sandwiched microspheres. A sample release chamber 2234 is also shown. Within the sample release chamber 2234 are a plurality of rotated inter-spacers 2236 (holding microspheres), a u-turn pipe 2238 for recycling unbound microspheres, a waste outlet 2240, a sample outlet 2242, and a light-emitting diode 2244.

The sample identification chamber 2246 includes microsticks 2248 for generating optical signals, and elution pool 2250, an elute outlet 2252, a fiber optical data link 2254 for converting electronic signals, and a signal amplifier 2256 which includes an organic light emitting diode. A sample pumping interface 2258 includes appropriate voltage regulators 2260, a parallel signal processor 2262, and pumps/flow meter 2264.

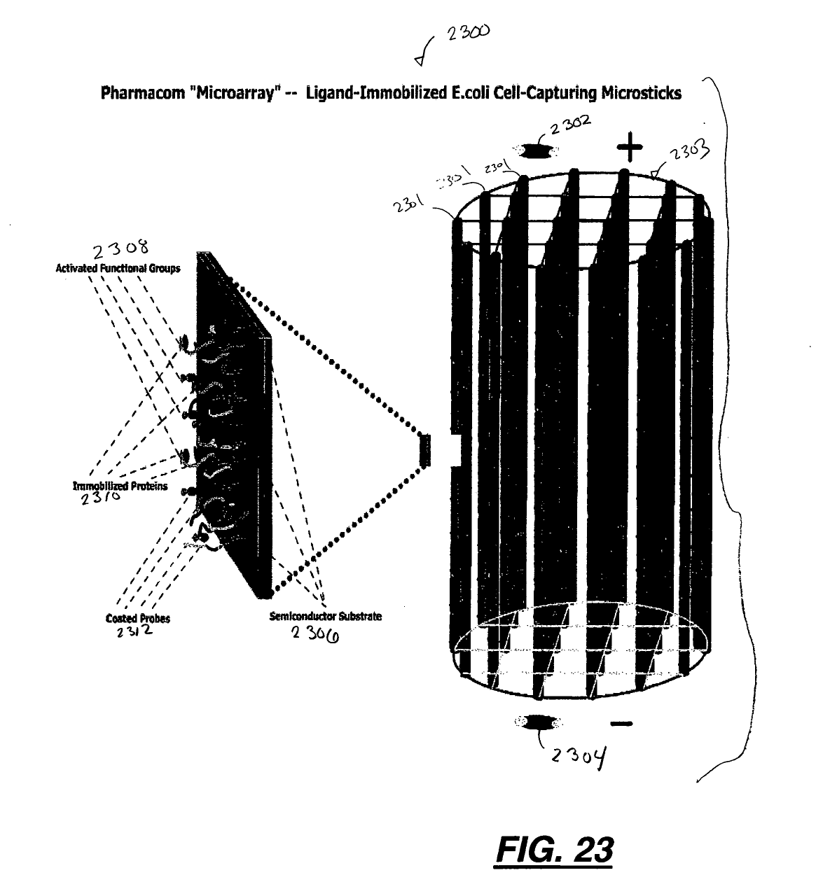

FIG. 23 illustrates one embodiment of a 3-dimensional compacted microarray (3-DCM). The microarray 2300 is an array of reactor-coated microsticks 2301 and is fabricated to have the potential to offer high-throughput detection of proteins, DNA, RNA, peptides and the entire cell respectively. One of the microsticks shows a semiconductor substrate 2306 with coated probes 2312, immobilized proteins 2310 and activated functional groups 2308. Each group of microsticks are coated with correspondent reactors, and “hosted” by each of unique microchambers 2303 that is designated to have a suitable micro-environment for an optimal molecule-molecule interaction. The self-contained microentity is called “microstick-in-column”. The microstick-in-columns can be resided in reagent-contented air or a reagent-contented liquid. The temperature outside or inside the column can be unjustified by thermal sensors, rapid thermal generators and system controlling software. Some of microstick-in-columns perform their electrostatic activity by joining with microelectrodes 2302, 2304 at two ends; some perform light transmission by linking to a miniature LED at terminal and a miniature OLED at another terminal. Multiple groups of the microstick-in-column are orchestrated by a number of integrated algorithms, a neural-network combinatorial code board and the parallel signal processor. The 3-DCM is designed to have the potential to offer high-throughput detection of proteins, DNA, RNA, peptides and whole-cell. They are able to facilitate simultaneous analysis of multiple samples for multiple analytes and improved measurement confidence through increased statistical data.

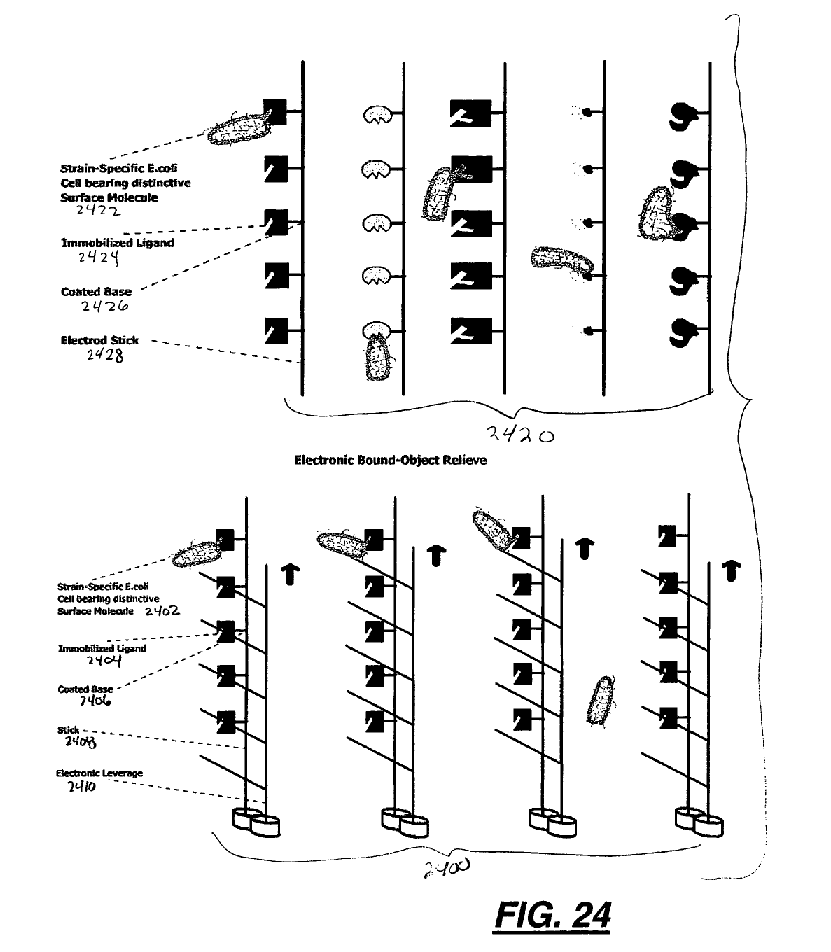

FIG. 24 illustrates one embodiment of the methodology for an object-capture and object relieve of the present invention. The methodology provides for autonomously disassociating the objects which were attaching to the receptors at microsticks. It allows a new target to be approached and bound from a subsequent wave of sample flow. In the process 2420, a strain-specific E. coli cell bearing a distinctive surface molecule 2422 is shown attached to an immobilized ligand 2426 attached to a coated base 2426 of an electrode stick 2428. The process 2400 shows a strain-specific E. coli cell 2402 bearing a distinctive surface molecule, an immobilized ligand 2404, a coated base 2406, a stick 2408, and electronic leverage 2410. Note how the electronic leverage 2410 is used to release the E. coli cell.

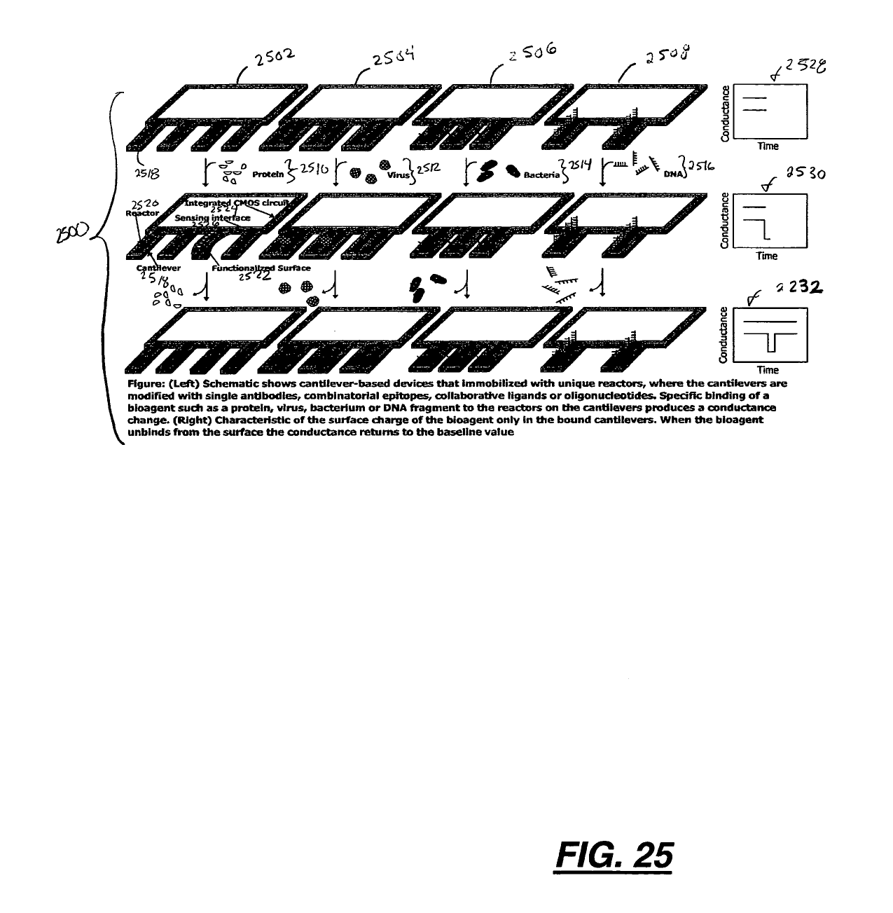

FIG. 25 illustrates one embodiment of an array of reactor-coated microsticks 2500. The array of reactor-coated microsticks 2500 are oscillations-based and nanowire-facilitated sensing elements. Each of devices 2502, 2504, 2506, and 2508 are cantilever-based devices immobilized with unique reactors. The cantilevers can be modified with single antibodies, combinatorial epitopes, collaborative ligands or oligonucleotides. Specific binding of a bioagent such as a protein 2510, virus 2512, bacterium 2514 or DNA fragment 2516 to the reactors 2520 on the cantilevers 2518 produces a conductance change. Each microstick can include an integrated CMOS circuit 2524 with a sensing interface 2526. Characteristics of the surface charge of the bioagent only in the bound cantilevers. When the bioagent unbinds from the surface the conductance returns to the baseline value. This is shown for the graphs 2528, 2530, and 2532.

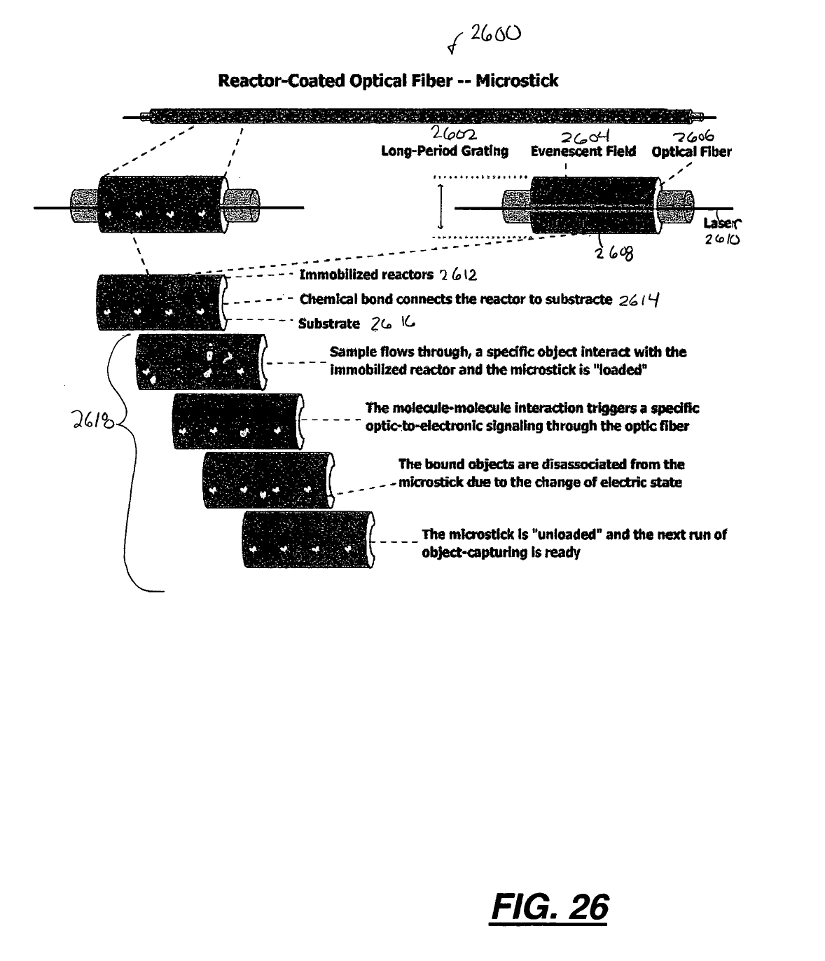

FIG. 26 illustrates one embodiment of a reactor-coated microsticks. Many essential features of a sensor such as small size, array format and cross reactivity are incorporated into microsticks—thin film-coated optical fibers. It carries excitation light produced by the light-emitting diodes through interior of an optic fiber. Excitation light produced by the blue light-emitting diodes passes through an optical fiber. Optical signals can be interrogated and collected at multiple wavelengths with different signal intensities, different phases, polarization and exited state lifetimes. An event of molecule interaction occurred at the surface of the optic fiber re-features the original patterns of the wavelength.

A microstick is a 3D optic fiber. A thin film membrane 2608 is coated at the surface of the fiber 2606. A semiconductor substrate 2616 lies at middle. Modulated light from the LED is launched and light travels through the cladding region—the interior layer of the fiber, and hits a photodetector at another end of the fiber. The output of the photodetector is processed by a digital-interface circuit board connected to the parallel input port of a computer. In essence, what one seeks to compute is the proportion of light reaching the photodetector as an indication of the amount (if any) of bioagent on the sensory layer. Any bioagent binds to the sensory layer affects the evanescent wave of the light propagating in the fiber. The effect is primarily a result of (1) a change in the index of refraction to which the evanescent wave is subject and (2) increased scattering of light. The evanescent wave is shallow enough that the microstick 2600 exhibits a significant response to the bioagent. The optical signals can be interrogated and collected at multiple wavelengths with different signal intensities, different phases, polarization and exited state lifetimes. The signals are directed to another end of the optical fiber where the variance of wavelength can be detected immediately or the energy can be further utilized by OLED.

FIG. 26 illustrates a long period grating 2602, an evanescent field 2604, an LED or laser light 2610. On the surface of thin film coating 2608 are immobilized reactors 2612, a chemical bond 2614 connecting the reactor 2612 to the substrate 2616. Process steps 2618 illustrate that when the sample flows through, a specific object interacts with the immobilized reactor 2612 and the microstick is then considered “loaded.” The molecule-molecule interaction triggers a specific optic-to-electronic signaling through the optic fiber. Then the bound objects are disassociated from the microstick due to the change of electric state. The microstick is then considered to be “unloaded” and the next run of object-capturing is ready.

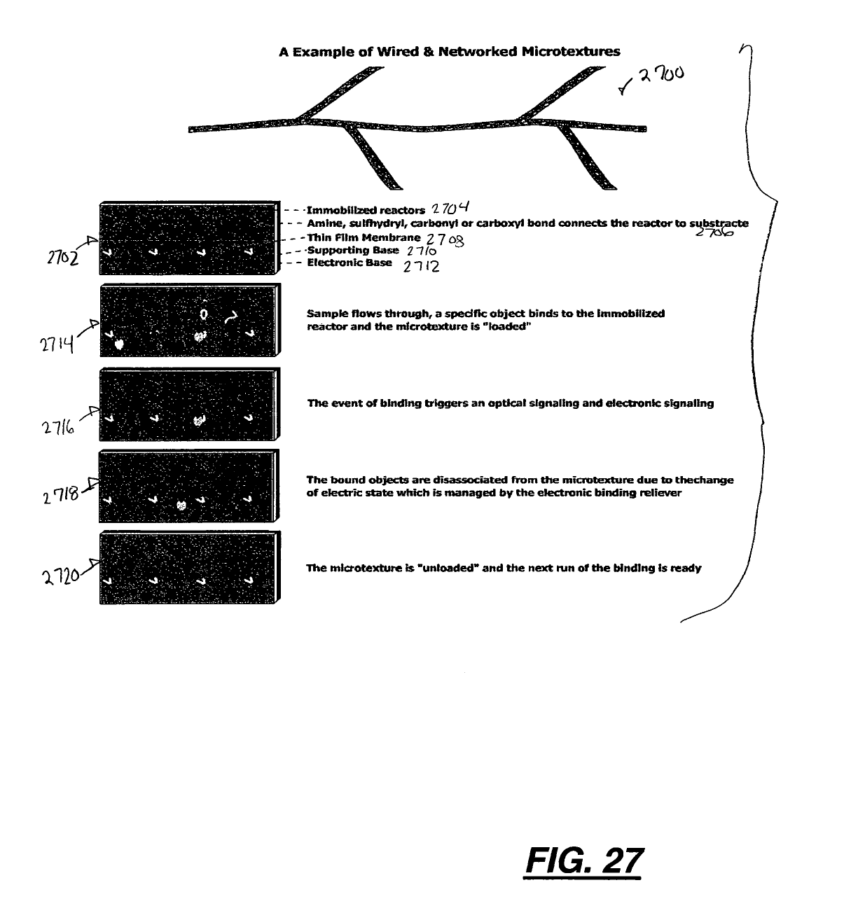

FIG. 27 illustrates one embodiment of CMOS-based, florescence-featured and OLED-supported sensing elements. As shown in FIG. 27 a wired or networked system 2700 of microtextures is shown. In step 2702, there is an electronic base 2712 overlaid by a supporting base 2710 which overlaid by a thin film membrane 2708. Immobilized reactors 2704 are shown. Amine, sulfhydrl, or carbonyl bond 2706 is used to connect a reactor to the substrate. In step 2714, the sample flows through and a specific object binds to the immobilized reactor and the microtexture is considered “loaded.” In step 2716, the event of binding triggers an optical signaling and electronic signaling. In step 2718, the bound objects are disassociated from the microtexture due to the change of electric state which is managed by the electronic binding reliever. In step 2720, the microtexture is “unloaded” and the next run of the binding is ready.

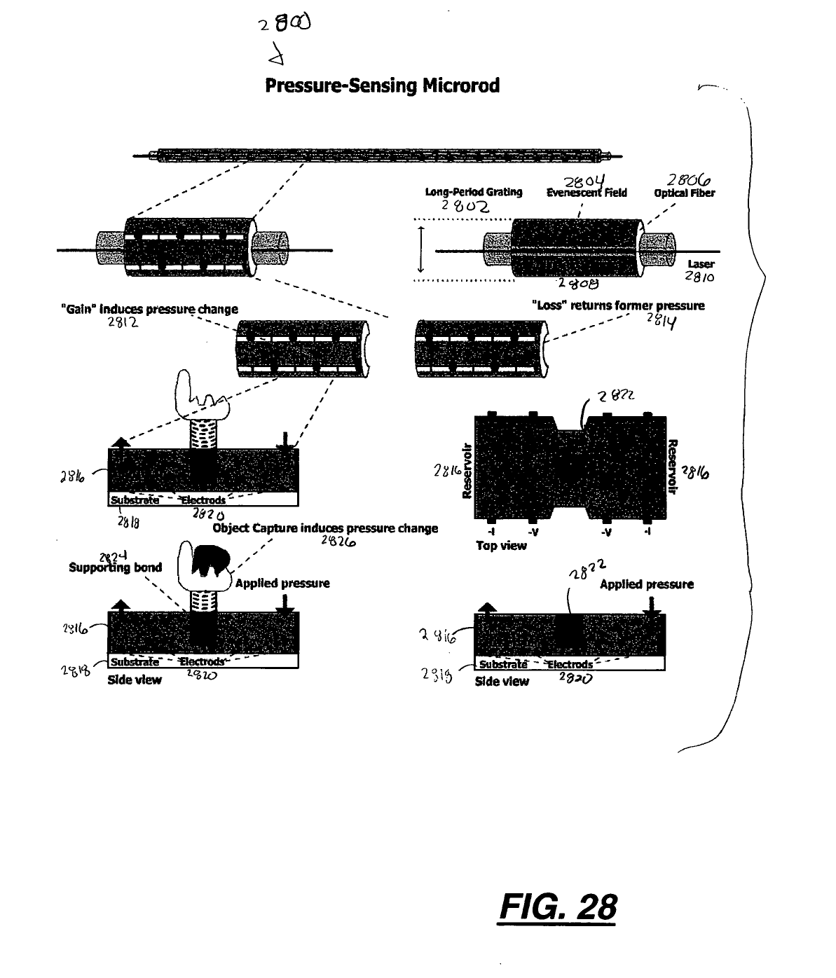

FIG. 28 illustrates one embodiment of pressure-based and electrostatic-mediated sensing elements. The pressure-sensing microrods are pressure-based and electrostatic-mediated sensing elements. The microrod 2800 shown is based on a binding triggered pressure. A long-period grating 2802 is shown with an evanescent field 2804, an optical fiber 2806, a thin film coating 2808, and a laser 2810. The capture of an object or “gain” induces pressure change as shown in step 2812. The “loss” returns the former pressure as shown in step 2814. Note there is a reservoir 2816, electrode 2820, substrate 2818. A pore 2822 is also shown.

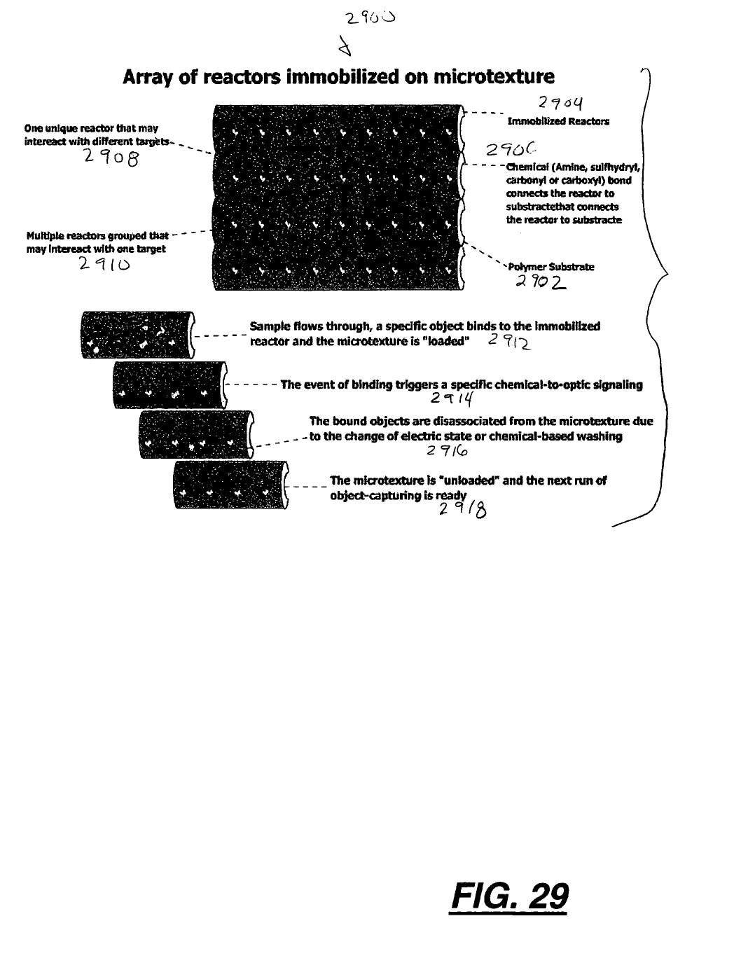

FIG. 29 illustrates an array of reactors 2900 immobilized on microtexture The array of reactors 2900 provide CMOS-based, thermal-featured and OLED-supported sensing elements. As shown in FIG. 26, there is a polymer substrate 2902. Immobilized reactors 2904 are shown on the substrate 2902. Chemical bonds such as amine, sulfhydryl, carbonyl, or carboxyl bonds connect the reactors to the substrate 2902. One unique reactor 2908 may interact with different targets. A group of multiple reactors 2910 may interact with one target.

As shown in step 2912, a sample flows through and a specific object binds to the immobilized reactor and the microtexture is now considered “loaded.” In step 2914, the event of binding triggers a specific chemical-to-optic signaling. In step 2916, the bound objects are disassociated from the microtexture due to the change of electric state or chemical-based washing. In step 2918, the microtexture is “unloaded” and the next run of object-capturing is ready.

The microtexture is a CMOS-based sensing element. Zinc 5,10,15,20- tetra phenylporphyrin (ZnTPP) (or other suitable chemicals) is selected as coating material in the sensor by immobilizing it on the surface of silicone rubber. Absorbance and fluorescence emission were the mode of detection. A spectral change occurs due to the co-ordination of NH3 molecules to the ZnII ion in the immobilized metalloporphyrins. They claim that such optical sensors have advantages over other sensors like immunity to electrical and electromagnetic interference, ruggedness, small size and their low cost and the problem such as less selectivity, signal drift over long periods can also be avoided by using these optical sensors with immobilized metalloporphyrins as sensing films. The thin film of porphyrins has been previously used for the detection of toxic gases based on fluorescent measurements. Sensing films made from the ZnTpp immobilized in silicone rubber were found to be the most sensitive for NH3 sensing.

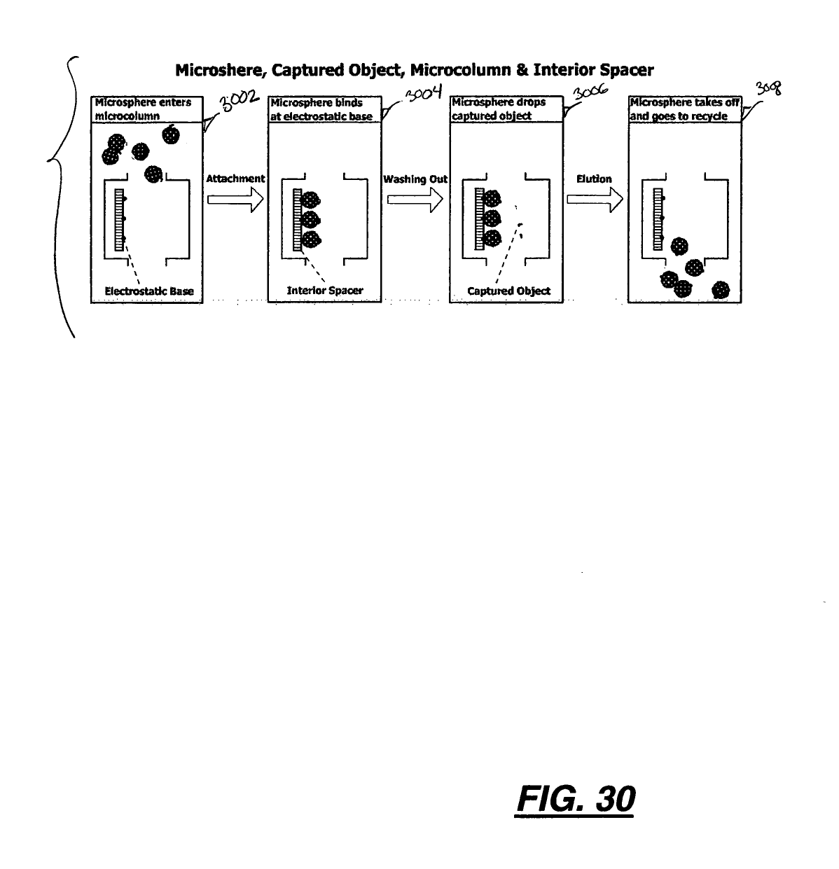

FIG. 30 illustrates one embodiment of a miniaturized laser setup of the present invention for the object capture of a microsphere-in-microcolumn. In FIG. 30, step 3002, a microsphere enters a microcolumn. In step 3004, the microsphere binds at an electrostatic base. In step 3006, the microsphere drops the captured object. In step 3008, the microsphere takes off and goes to recycle. Interspacers inside the microcolumn are made of electrostatic membranes. The membrane interacts with and binds to entered microspheres based on an electrostatic mechanism. The object which are carried by a microsphere is stripped away from the microsphere through the first elution, and the microsphere itself remains attachment with the membrane. The naked microsphere will be held by the membrane temporally until the second elution. Three different micropipes are implemented here. Each three pipes are connected with a microcolumn at the bottom. The first pipe leads eluted samples from the container into analytical chambers, the second pipe delivers waste to an outlet after elution, and the third pipe pumps stripped microspheres back to the up-level for next run. The micro-chromatography steps involve: (1) samples from the Sample Fluidifier entered into an pre-equilibrated affinity chromatography microcolumn; (2) the interior spacer temporally holds object-carrying microspheres based on electrostatic force; (3) the attached microspheres remains attachment with the interior spacer but the objects which microspheres were carrying are eluted by changing pH value and organic solvent concentration; (4) the unbound microspheres are released from the microcolumn by adjusting electrostatic strength.

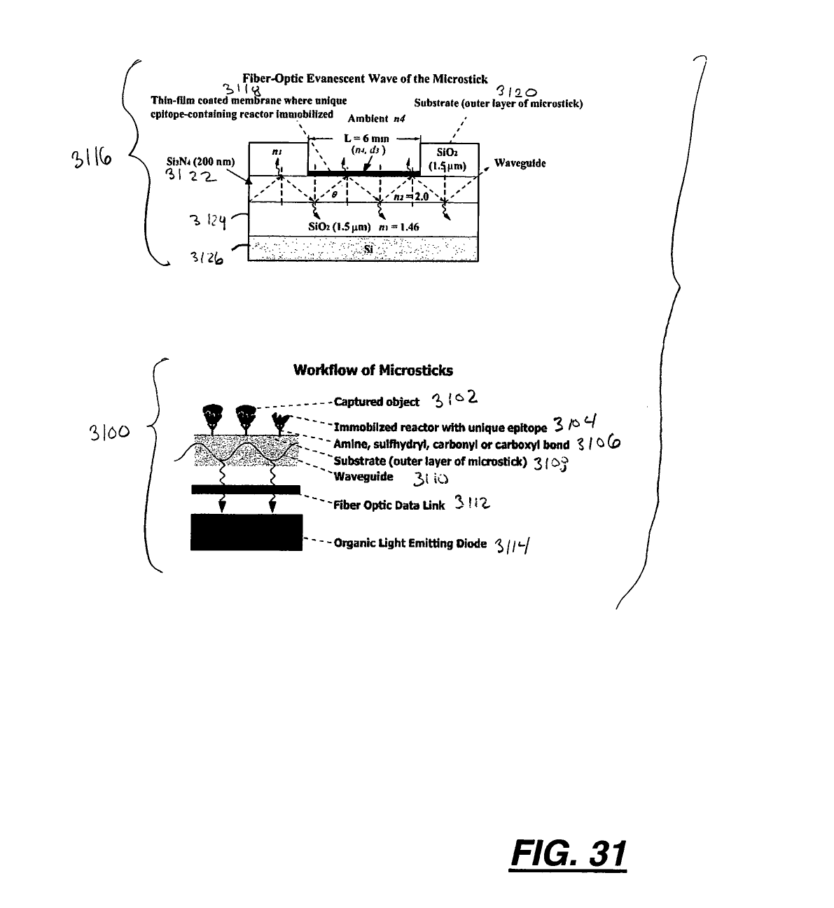

FIG. 31 illustrates another embodiment of the present invention showing the workflow of microsticks, including the functional layers of the microstick and the configuration of the waveguide on a silicon substrate of the microstick. The microstick 3100 is shown with a captured pathogen 3102, an immobilized reactor 3104 with unique epitope. An amine, sulfhydryl, carbonyl or carboxyl bond 3106 is also shown connecting the immobilized reactor 3104 to a substrate 3108 which forms an outer layer of the microstick. A waveguide 3110 is shown, as is a fiber optic data link 3112 and an organic light emitting diode 3114. A cross-section 3116 illustrates a thin-film coated membrane 3118 where unique epitope-containing reactor is immobilized with the substrate 3120 on either side. A layer 3122 is shown which serves as a waveguide. A layer 3124 such as formed by SiO₂ is also shown. A layer of silicon 3126 is also provided.

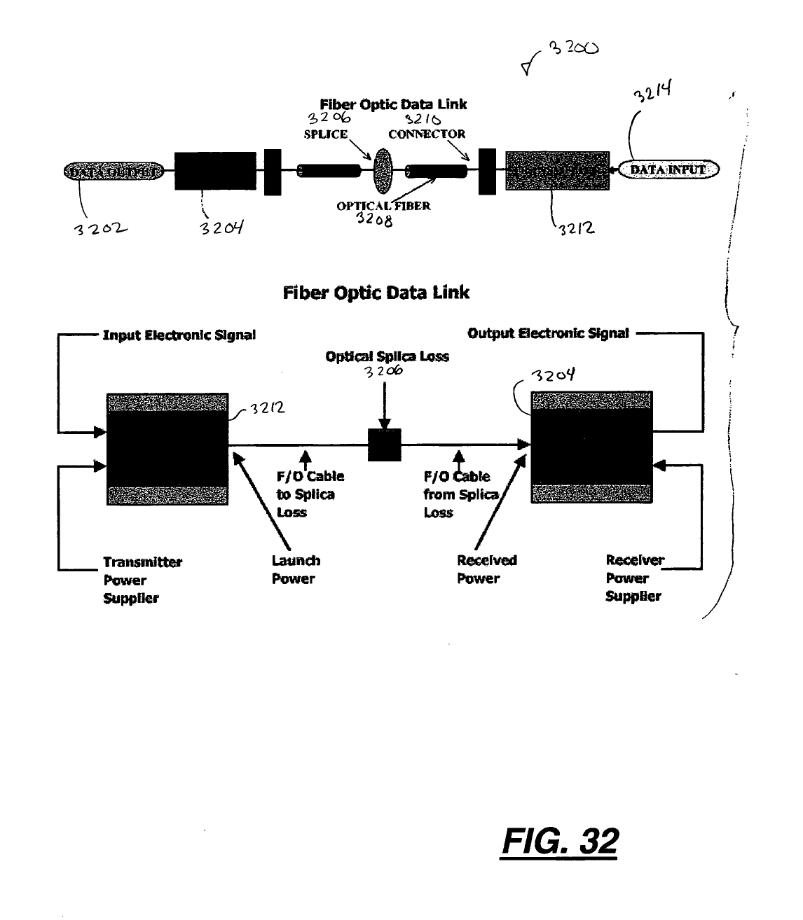

FIG. 32 illustrates one embodiment of a fiber optic data link of the present invention. A data input 3214 is operatively connected to a transmitter 3212 which is operatively connected through a connector 3210 to an optical fiber 3208 which is connected through a splice 3206 to a receiver 3204 and then to a data output 3202. The fiber optic data link is implemented for enabling the microsystem for remote communication. The transmitter, optical fiber, and receiver perform the basic functions of the fiber optic data link. Each part of the data link is responsible for the successful transfer of the data signal. The transmitter is needed to effectively convert an electrical input signal to an optical signal and launch the data-containing light down the optical fiber. The receiver is needed to effectively transform this optical signal back into its original form. The electrical signal provided as data output needs to exactly match the electrical signal provided as data input. The transmitter converts the input signal to an optical signal suitable for transmission.

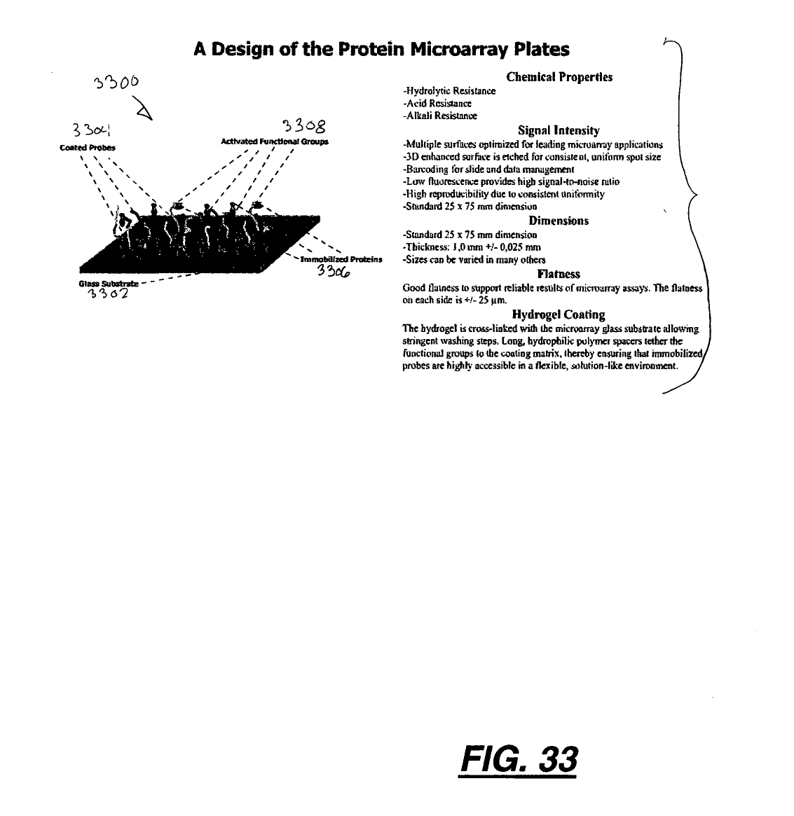

FIG. 33 illustrates one embodiment of a design for protein microarray plates. The microarray plate 3300 shown includes a glass substrate 3302. Coated probes 3304 are disposed on the glass substrate 3302. Immobilized proteins 3306 are shown with activated functional groups 3308. The microarray plate 3300 has desirable chemical properties such as being hydrolytic resistant, acid resistant, and alkali resistant. Preferably, the microarray plate has multiple surfaces optimized for leading microarray applications, a 3D enhanced surface etched for consistent, uniform spot size, barcoding for slide and data management, low fluorescence to provide high signal-to-noise ration, high reproducibility due to consistent uniformity, and standardized dimensions. One standardized dimension that can be used is 25 mm×75 mm with a thickness of 1.0 mm+/−0.025 mm. Of course, the size can be varied. Preferably the microarray plate 3300 provides good flatness to support reliable results of microarray assays. The flatness on each side is +/−25 um. A hydrogel coating is preferably used which is cross-linked with the microarray glass substrate 3302 allowing stringent washing steps. Long, hydrophilic polymer spacers tether the functional groups to the coating matrix, thereby ensuring that immobilized probes are highly accessible in a flexible, solution-like environment.

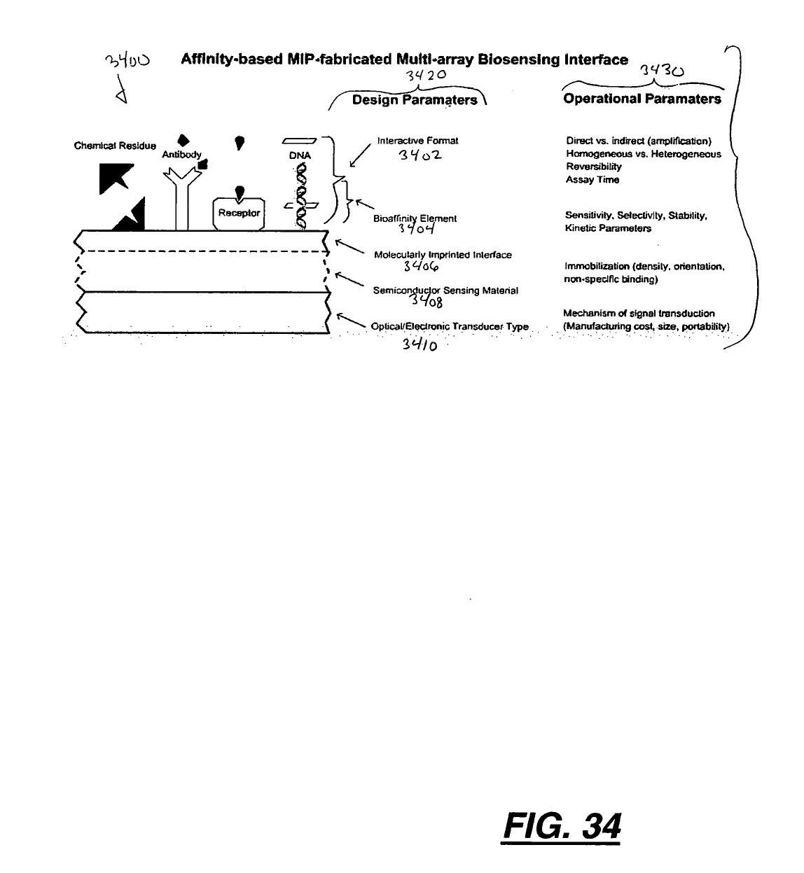

FIG. 34 illustrates an affinity-based MIP-fabricated multi-array biosensing interface 3400. Both design parameters 3420 as well as operational parameters 3430 are shown. Note that the design parameters 3420 include an interactive format 3402, a bioaffinity element 3404, a molecularly imprinted interface 3406, a semiconductor sensing material 3408, and an optical/electronic transducer type 3410. The multiple detective interfaces of the present invention are designed to have the potential to offer high-throughput detection of proteins, DNA, RNA, peptides and whole-cell. They are able to facilitate simultaneous analysis of multiple samples for multiple analytes and improved measurement confidence through increased statistical data.

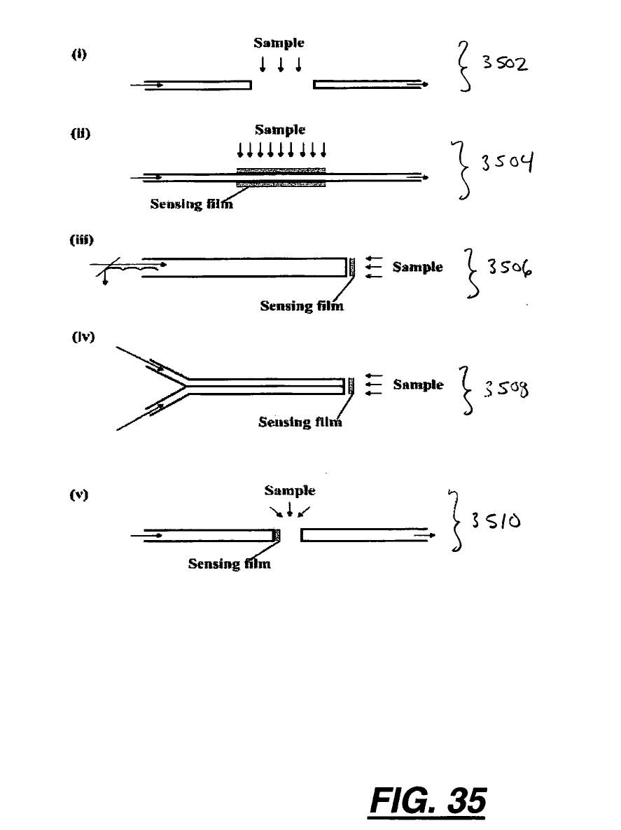

FIG. 35 illustrates different configurations of light sources that can be used in various embodiments of the present invention. A first configuration 3502, a second configuration 3504, a third configuration 3506, a fourth configuration 3508, and a fifth configuration 3510 are shown. Thus, it should be clear that there are various light source settings that can be used in various embodiments of the present invention. A miniature light source can be set up according to particular needs of each microsystem.

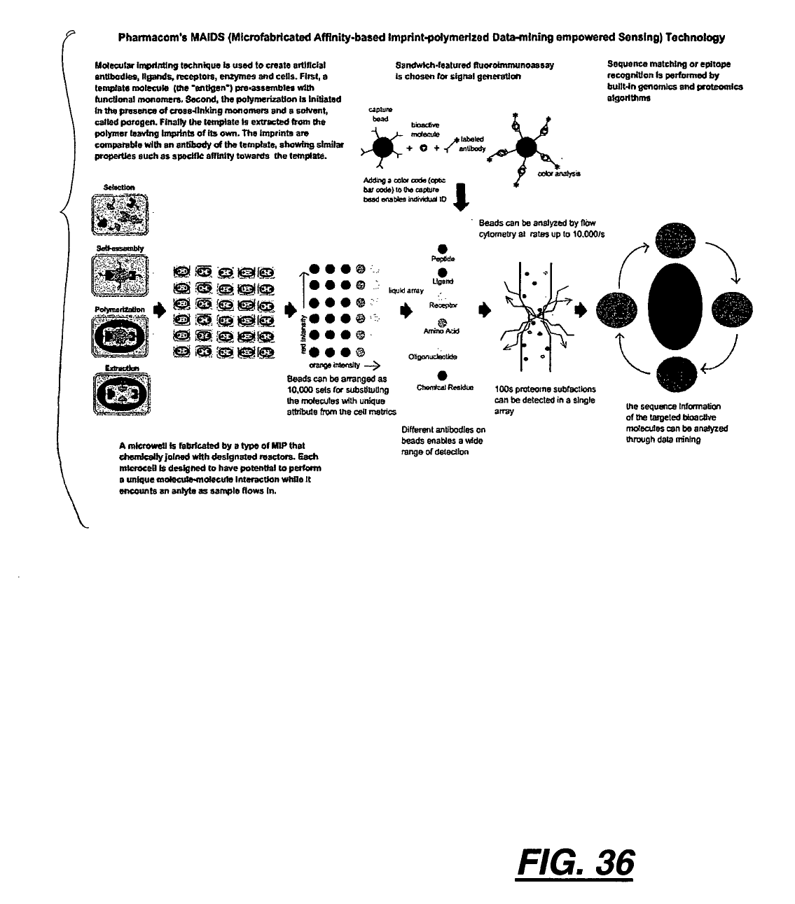

FIG. 36 demonstrates the MAIDS (Microfabricated Affinity-based Imprint-polymerized Data-mining empowered Sensing) Technology. Molecular imprinting technique is used to create artificial antibodies, ligands, receptors, enzymes and cells. First, a template molecule (the “antigen”) pre-assembles with functional monomers. Second, the polymerization is initiated in the presence of cross-linking monomers and a solvent, called pathogen. Finally the template is extracted from the polymer leaving imprints of its own. The imprints are comparable with an antibody of the template, showing similar properties such as specific affinity towards the template. A microwell is fabricated by a type of MIP that chemically joined with designated reactors. Each cell metrics is designed to have potential to perform a unique molecule-molecule interaction while it encounters an analyte as sample flows in. Sandwich-featured fluoroimmunoassay is chosen for signal generation. Sequence matching or epitope recognition is performed by built-in genomics and proteomics algorithms.

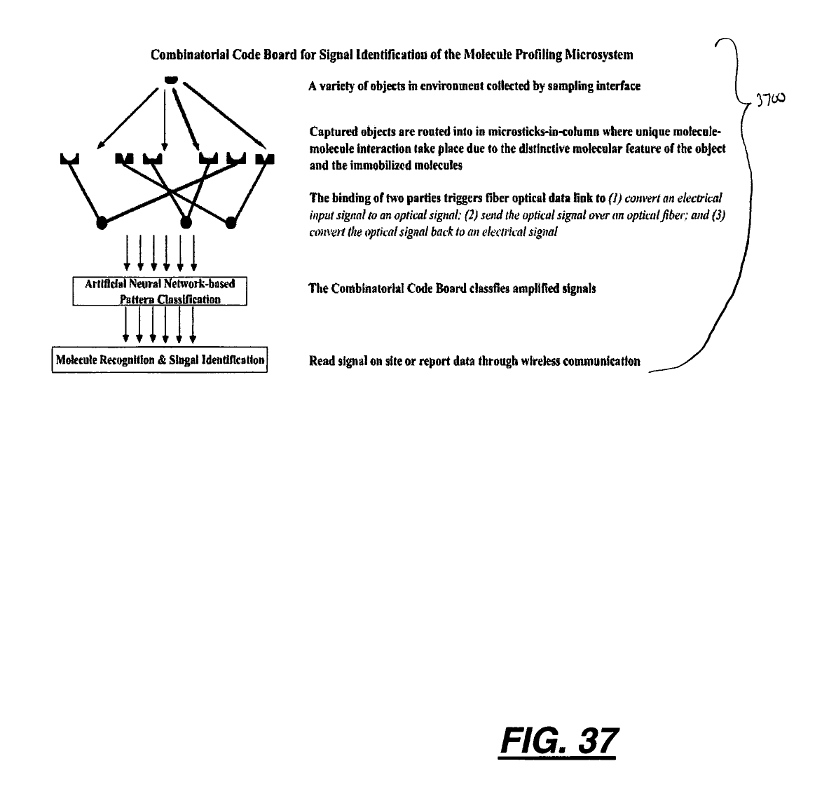

FIG. 37 illustrates one embodiment of a neural network algorithm-based combinatorial code board 3700 according to one embodiment of the present invention. The neural network algorithm-based combinatorial code board 3700 works as the core part of the parallel signal processor, which allows orchestrating data flow generated from hundreds of samples and their signaling channels simultaneously. It leads the workflow of the multiple target recognition and the multiple channel signal reporting. (1) Multiple molecules to be detected in an open environment; (2) Microarray of the molecules featured with distinctive motifs that will cause unique antigen/antibody interaction; (3) The detected molecules react with a built-in enzyme-based reporting system and the chemical reaction triggers electronic signal; (4) Neural-Network-based Pattern Classification; (5) The Combinatorial Code Board for interpreting and classifying the amplified signals; (6) Molecule Recognition and Signal Identification; (7) Signal reading and data reporting through wireless communication.

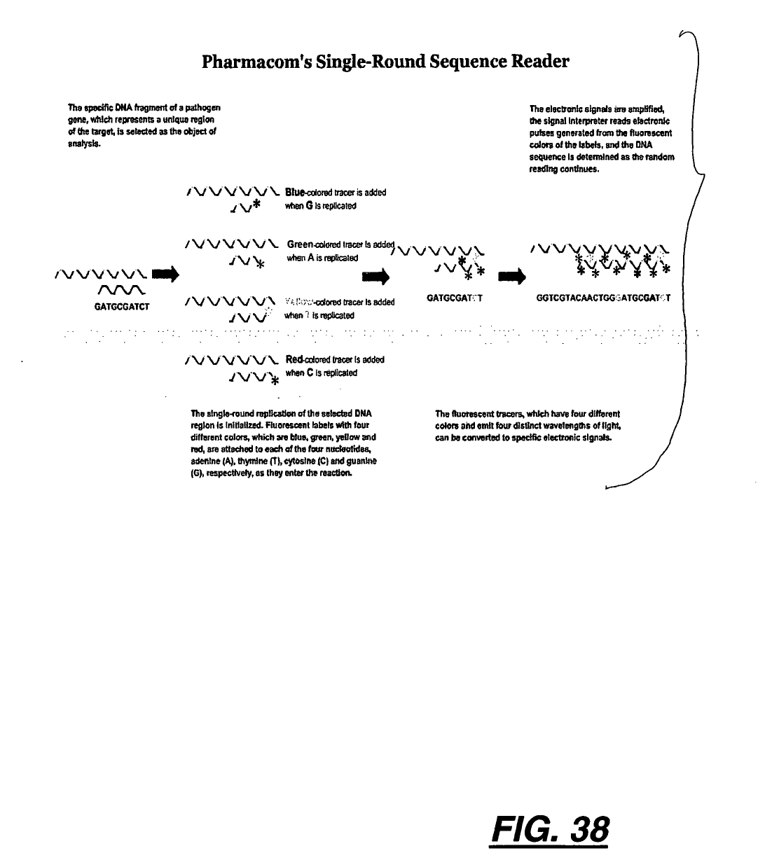

FIG. 38 demonstrates one embodiment of the procedure for single-round sequence reading and signal generation. Each of the four nucleotides is labeled with four different fluorescent tags and the resulting fluorescent signals with their different wavelengths are converted to specific electronic signals. The cascade of the overall reaction with respect to analysis of DNA consists of the following steps: (I) The specific DNA fragment of a pathogen gene, which represents a unique region of the target, is selected as the object of analysis; (ii) The single-round replication of the selected DNA region is initialized. The four nucleotides, adenine (A), thymine (T), cytosine (C) and guanine (G) are labeled with fluorescent tags with four different colors, which are green, yellow, red and blue, respectively, as each nucleotide enters the reaction; (iii) The fluorescent tracers, which have four different colors and emit photons with four distinct wavelengths of light; (iv) A photon with a certain wavelength strikes a light-sensitive material and kicks out a single electron which then instigates an avalanche of millions of electrons in a kind of sparking process within a microvacuum tube; (v) Once it is excited by absorption of a photon, the electron can leap onto the terminal of a single-electron transistor, where it “throws the switch” and is detected. The electronic signal can be measured using a nanoscale electron counter.

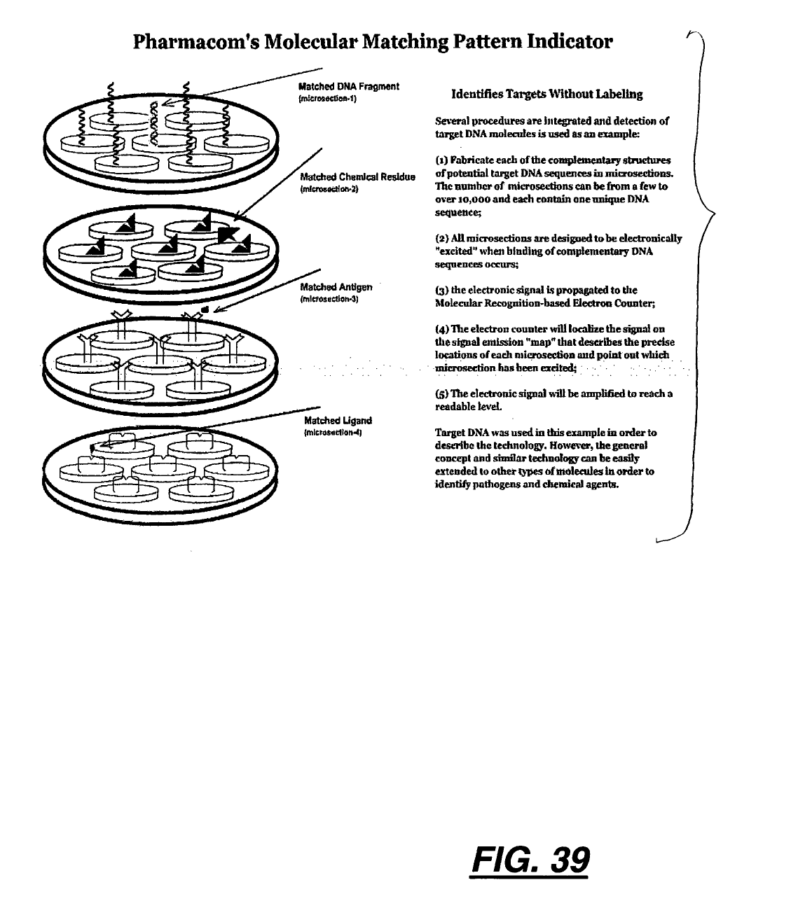

FIG. 39 demonstrates the Molecular Matching Pattern Indicator according to one embodiment of the present invention. The turnplate-featured technology uses color to read sequences: (1) the complementary structures of potential target DNA sequences are immobilized in the metrics of microwells. The number of microwells can be from a few to over 10,000 and each can contain one unique DNA sequence; (2) All microwells are designed to be electronically “excited” when binding of complementary DNA sequences occurs; (3) Once it is excited by the absorption of a photon which is designed to be resulted from a perfect molecular matching, the electron leaps onto the terminal of the single-electron transistor, where the electronic signal is propagated to the Molecular Recognition-based Electron Counter; (4) The Counter will localize the signal on the signal emission “map” that describes the precise locations of each microwell and point out which microwell has been excited; (5) The electronic signal will be amplified to reach a readable level. Although target DNA was used in this example in order to describe the technology, the technology can be easily extended to other types of molecules in order to identify bioagents.

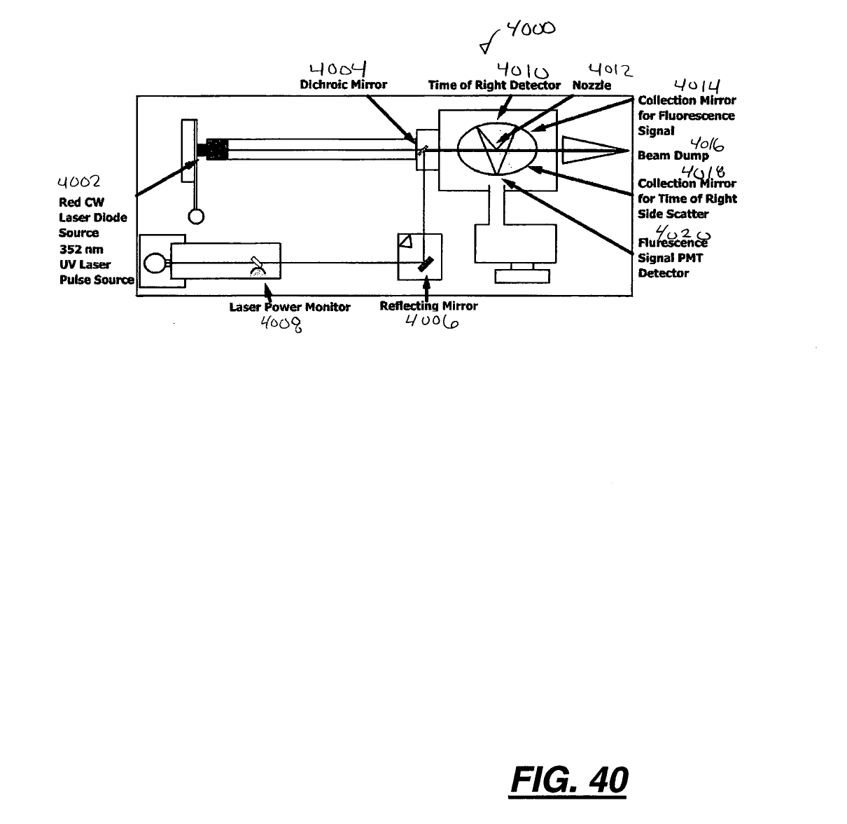

FIG. 40 demonstrates a miniaturized laser setup according to one embodiment of the present invention. As shown in FIG. 40, the laser setup 4000 includes a red CW laser diode source 4002 which is directed towards a dichroic mirror 4004. A time of right detector 4010 is shown. A collection mirror 4014 for fluorescence signal is provided with a beam dump 4016. There a collection mirror 4018 for time of right side scatter and a fluorescence signal PMT detector 4020. A UV laser pulse source 4003 is shown with a laser power monitor 4008 and a reflecting mirror 4006 in alignment with the reflecting mirror 4006.

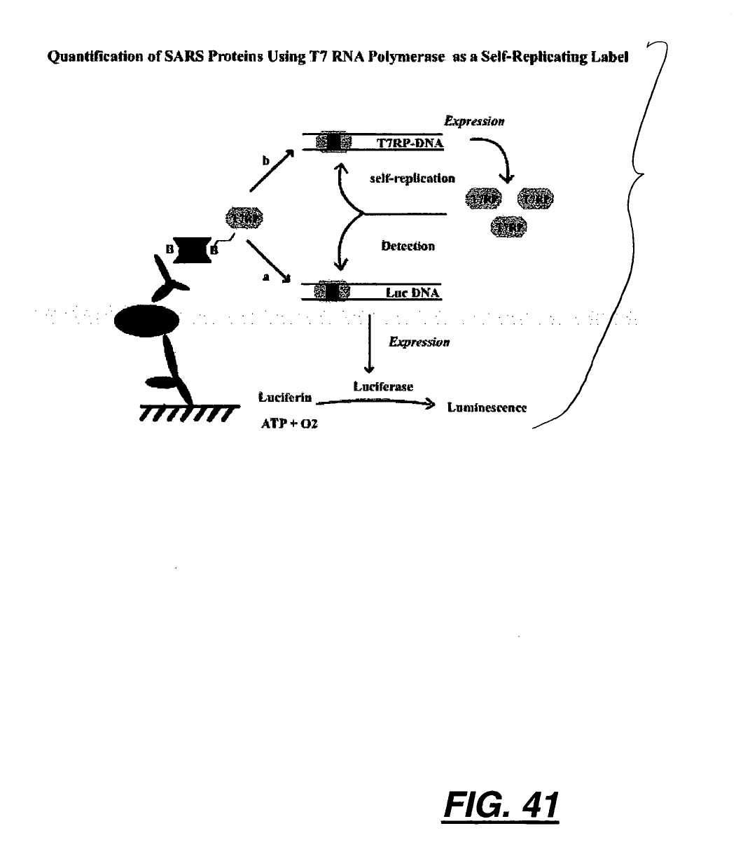

FIG. 41 demonstrates one embodiment of the integrated IDAT and T7RP-SRLPQ assay. An antigen is bound simultaneously to an immobilized capture antibody and a biotinylated detection antibody. BT7RP complexed with streptavidin (SA) is then added to the immunocomplex. The bound T7RP is determined by in vitro coupled transcription/translation. Two approaches will be explored. (a) T7RP acts on firefly Luc-DNA, located downstream of the T7 promoter, to produce several molecules of active luciferase which is measured by its characteristic bioluminogenic reaction. (b) T7RP acts on T7RP cDNA (T7RP-DNA), positioned downstream of the T7 promoter, to generate several T7RP molecules (self-replication phase) which, in turn, act on Luc-DNA to produce luciferase (detection phase). B, biotin. The T7 promoter is represented by a hatched square as the figure above.

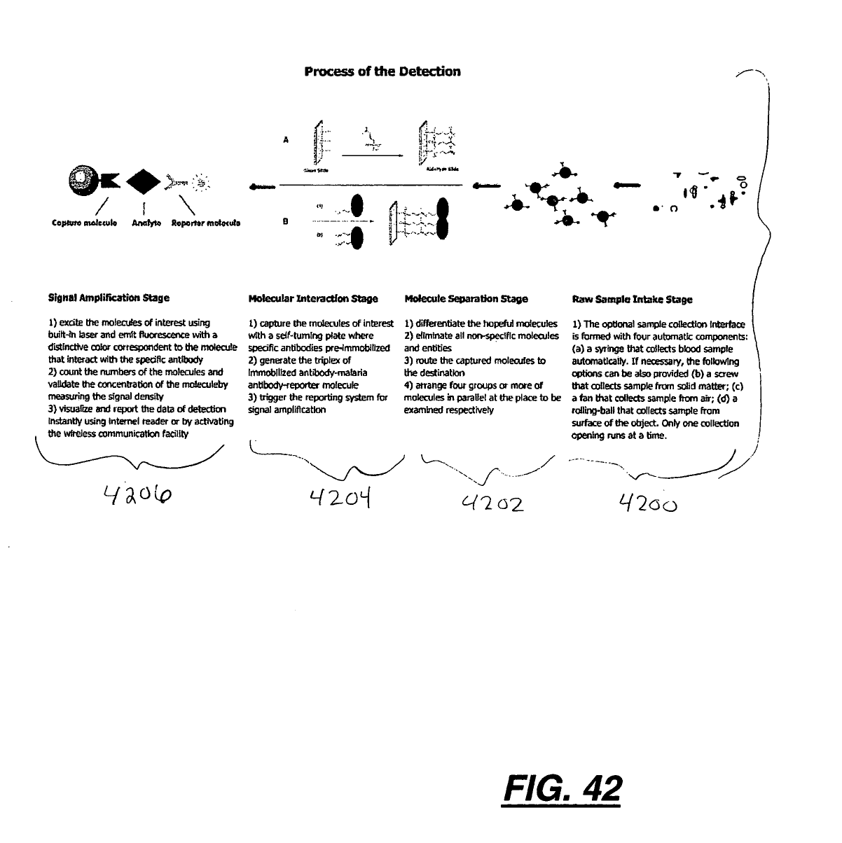

FIG. 42 illustrates the stages of detection according to one embodiment of the present invention. The stages include a raw sample intake stage 4200, a molecule separation stage 4202, a molecular interaction stage 4204, and a signal amplification stage 4206. In the raw sample intake stage 4200, the optional sample collection interface is formed with four automatic components: (a) a syringe that collects blood sample automatically. If necessary, the following options can be also provided (b) a screw that collects sample from solid matter; (c) a fan that collects sample from air; (d) a rolling-ball that collects sample from surface of the object. Only one collection opening runs at a time.

The molecule separation stage 4202 provides for 1) differentiating the targeted molecules; 2) eliminating all non-specific molecules and entities; 3) routing the captured molecules to the destination; and 4) arranging four groups or more of molecules in parallel at the place to be examined respectively.

The molecular interactions stage 4204 provides for 1) capturing the molecules of interest with a self-turning plate where specific antibodies pre-immobilized; 2) generating the triplex of immobilized antibody-malaria antibody-reporter molecule; and 3) triggering the reporting system for signal amplification.

The signal amplification stage 4206 provides for 1) exciting the molecules of interest using built-in laser and emit fluorescence with a distinctive color correspondent to the molecule that interact with the specific antibody; 2) counting the numbers of the molecules and validate the concentration of the molecule by measuring the signal density; 3) visualizing and reporting the data of detection instantly using internal reader or by activating the wireless communication facility.

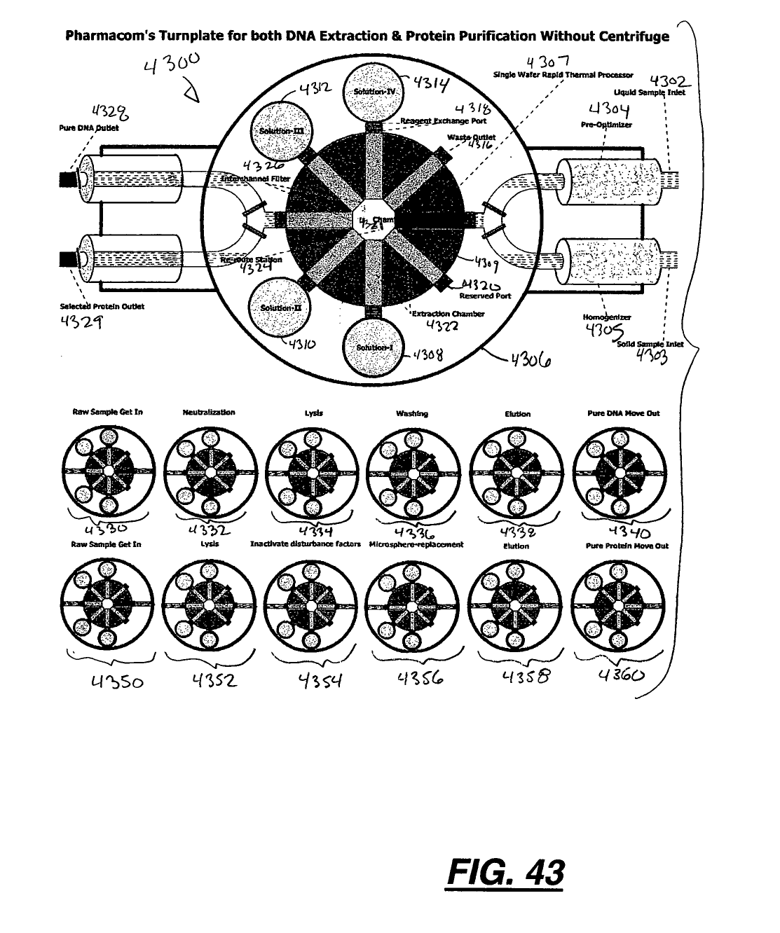

FIG. 43 illustrates one embodiment of a turnplate system 4300 for DNA extraction and protein purification at a microscale without a centrifuge. The turnplate system 4300 is an automated platform that is built at integrated circuits and coordinated by a central microprocessor. It contains five types of microdevices. The homogenizer 4305 that uses a glass beads is coupled to disrupt cellular materials through abrasion. The resulting pulp is used for DNA analysis or protein isolation. The Single Wafer Rapid Thermal Processor 4307 facilitates measurable and well-controlled thermal changes while each reaction chamber turns to be its designated operation. The reagent suppliers (4308, 4310, 4312, 4314) inject solutions into reaction chamber 4321 according to a pre-defined time-table. The reaction chambers 4321 host processes of digestion, catalysis, dilution, washing, elution or others. The waste collector sucks solution from the reaction chamber 4321 when its port 4316 switches over.

The turnplate 4306 moves clockwise starting from raw sample pumping-in to pure DNA pumping-out during the extraction. In a first step, blood drops, cells, leaf punches or other liquid or solid materials in small quantity are placed into the “Raw Sample Inlet” 4302. In a second step, the sample is moved into the pre-optimizer 4304 that contains PCR-compatible lysis buffer. In a third step, a single wafer rapid thermal processor 4307 heats up the pre-optimizer 4304. In a fourth step, the sample is pumped into the turnplate 4306. In a fifth step, the lysate is routed into the “Solution Chamber-I” 4308 through a filtered gate for neutralization. In a sixth step, the lysate is routed into the solution chamber-II 4310 through a filtered gate for dilution. Note that an interchannel filter 4326 is shown. In a seventh step, the lysate is routed into the solution chamber-III 4312 through a filtered gate for clean-up. In an eighth step, the lysate is routed into the solution chamber-IV 4314 through a filtered gate for elution. In a ninth step, pure DNA is pumped into the DNA outlet 4328. Next, pure DNA enters the phases of Real-Time PCR for signal generation.

Note that reserved port 4320 is shown as well as reagent exchange ports 4318. Also a waste outlet 4316 is provided. There is an extraction chamber 4322 connected to each port. A re-route station 4324 is shown in the center of the turnplate 4306.

Step 4330 shows the raw sample being received, step 4332 shows a neutralization step, step 4332 shows a lysis step, step 4336 shows washing, step 4338 shows elution, and step 4340 shows pure DNA moving out. Thus, the system and process for DNA extraction without a centrifuge allows for a raw sample taking at the raw sample inlet 4302 to be processed to provide pure DNA at the pure DNA outlet 4338.

The turnplate system 4300 can also be used for protein purification. For protein purification, the sample in small quantity is placed in the solid sample inlet 4303 and enters the homogenizer 4305. The lysate is routed into the solution chamber-I 4308 through a filtered gate for lysozyme and EDTA. The lysate is routed into the solution chamber-II 4310 through a filtered gate for inactivation of interfering substances. The lysate is routed into the solution chamber-III 4312 through a filtered gate for microsphere-based isolation. The lysate is routed into the solution chamber-IV 4314 through a filtered gate for elution. Candidate proteins are pumped into the selected protein outlet 4329. Next, the target protein enters the phase of bioaffinity-based signal generation.

The step of the raw sample entering for protein purification is shown in step 4350. The step of lysis is shown in step 4352. The step of inactivate disturbance factors is shown in step 4354. The step of microsphere replacement is shown in step 4356. The step of elution is shown in step 4358. The step of pure protein moving out is shown in step 4360.

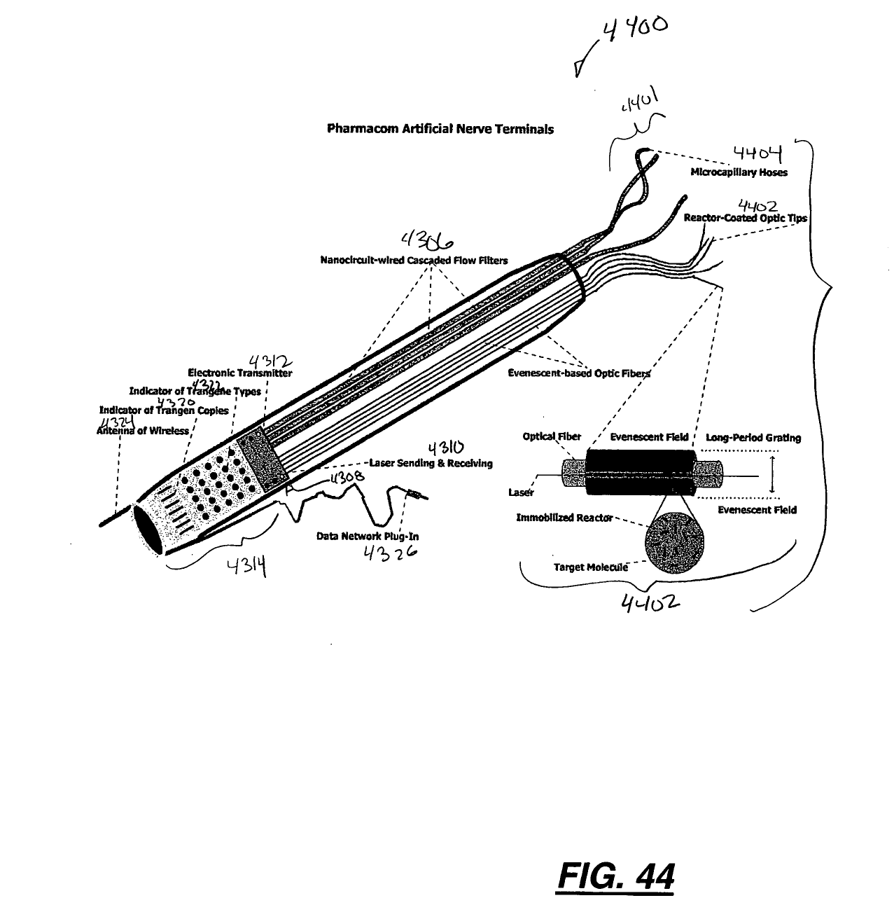

FIG. 44. illustrates one embodiment of artificial nerve terminals (ANT) 4400. ANT is a second form of plant GMO detectors that preferably comprises eight components situated at four nodes. The first node 4401 included the detection tip 4402 and aspiration hose 4404. The second node 4306 includes the sample filter and flow cascade. The third node 4308 is the laser station 4310 and electric center. The fourth node 4314 is the message reader and signal transmitter.

Node-I (4401): Detection Tip and Aspiration Hose. It contains a branch of tips. It contains two types of tips: (1) the tip coated with thin-film membrane and proper reactors that directly interfaces with plant liquid. Nanowire that has sensitive conductivity is implemented under the membrane. Many tips which are made up with different reactors can be used to target different objects or a same object in a time sequence. (2) the tip looks like a microcapillary that sucks small quantity of liquid sample from plant objects within a distance. Nanowire that has sensitive conductivity is joined with each nodes of the polymers. Many tips which are made up with different filtering polymers can be used to obtain different qualities of liquid samples.

Node-II (4306): Sample Filter and Flow Cascade. They are formed as a branch of extendable and flexible pipes. It contains two types of pipes. The first type of pipe is where the two-way optical fibers lie and the light from projected from the Laser station goes through one line of optical fibers and brings back signals from the reactor-coated tip through another line of optical fibers. The second type of pipe is the pipe in which samples with distinctive physiochemical properties are filtered through polymers, carried by different groups of microsphere and transferred from the capillary tip to another direction based on mechanical, optical, or electrokinetic forces which is involved subsequentially following phases of the movement. Samples are neutralized, digested, step-by-step eliminated in cascaded polymer sections and targeted analytes reach their destination where the reactions of biocatalyst, bioaffinity or hybridization occur.

Node-III (4308): Laser Station and Electric Center. They are two stand-alone units but bridged together through an interface. The laser station 4310 projects laser light through optical fibers to the tips and carries scattered lights back to the station. The electric center monitors events that the nanowire network has encountered and filters signals at the center.

Node-IV (4314): Message Reader and Signal Transmitter. They perform two different tasks based on different mechanisms. The message reader displays the signals right at the handle. This can include an indicator of transgene types 4322 as well as an indicator of transgene copies 4320. The signal transmitter 4312 transfer the signals between the device and remote databases through wireless communication. An antenna 4324 is shown as well as a data network plug-in 4326.

Having now illustrated a number of specific embodiments of the present invention, the flexibility of the present invention in numerous applications should be readily apparent. It should also be apparent that the present invention provides for different aspects of the embodiments shown to be substituted with other aspects of other embodiments shown.

According to one aspect, the present invention includes a merged system of microarray and microfluidics, empowered by a dual mode of genomic and proteomic processing, the workflow of the Microsystems is coupled with four stages in a streamline.

1) Stage of Sample Selection & Collection. Suitable technologies are chosen for enable the process: (i) Solid Phase Microextraction; (ii) Steam Distillation; (iii) Ultrasonic Rupture; (iv) Subcellular Fractionation; (v) Cell Cycle Synchronization (vi) Permeabilization; (viii) Microcapillaries; and Single Cell Extraction Without Centrifuge.

2) Stage of Sample Separation & Diffusion. Suitable technologies are chosen for enable the process: (i) Recombinant Antibody Phage Display; (ii) Microfluidic electroporation; (iii) Microsphere-facilitated biocatalyst; (iv) Microsphere-facilitated bioaffinity; and (v) Microsphere-facilitated hybridization.

3) Stage of Detection & Signaling. Suitable technologies are chosen for enable the process: (i) Single-cell/molecule-based Flow Cytometry; (ii) Self-Replicating Label for Protein Quantification; (iii) Immuno-Detection Amplified by T7 RNA Polymerase; (iv) Fluorescence Quenching; (v) Evanescent Scattering; (vi) Surface Plasmon Resonance; (vii) (7) Fluorescence-Activated Cell Sorting; (viii) Cantilever Oscillation, and (ix) Single Cell-based Real-Time PCR

4) Stage of Data Mining & Reporting. Suitable technologies are chosen for enable the process: (i) Reduced Instruction Set Computers; (ii) Neural-Network Combinatorial Code board; (iii) Wireless Interface; and (iv) Global Genomic/Proteomic databases.

The advanced features and functions of the microsystems include: (i) ability to identify a variety of bioagents with high sensitivity and selectivity; (ii) ability to analyze samples in multiple environments or metrics; (iii) ability to detect all classes of biological entities (include molecule residue, molecule, molecule complex, cellular faction, cell, bacterium, and virus); (iv) ability to perform multiplexed detection (at least 10 and up to 200 bioagents simultaneously); (v) minimal requirement for operator training; (vi) minimal requirement for external sampling process required; (vii) no special storage or set-up requirements. The system enables transporting the entire traditional biological detection laboratory to a portable device and offers significant advantages in terms of speed, efficiency, cost, use of small sample sizes and automation.

The innovation is reflected in the following aspects: (1) dynamic merge of distinctive science fields; (2) architecture of the dual mode system for both genomic test and proteomic test; (3) building blocks of principal components; (4) strategies of self-sampling, preconcentration, fluidization and microflow cytometry; (5) procedures of sensing element fabrication and surface molecule immobilization; (6) miniaturized Laser setup and optic component integration; (7) workflow of signal generation, processing and reporting; (8) implementation of software for general system operation and subcomponent manipulation; (9) extendable applications of the microsystem; and (10) attributes and features of critical microdevices in the microsystem. This innovative technology will have an enormous impact on the way DNA, RNA, protein, bacterial, and viruses and all rest of bioentities are collected, detected, analyzed and reported.

To further aid in understanding the invention, different possible subsystems of various embodiments are now discussed in detail. These include: (1) the system architecture; (2) the LOSM platform; (3) the “living” reactors; (4) the “micromachinery” reactors; (5) reactor-coated membranes; (6) immobilization of the reactors; (7) kinetic tuning of the microenvironment for reactor-target interaction; (8) dynamic sample collection interfaces; (9) the autonomous reagent supplier; (10) single cell extraction without centrifugation; (11) RNA polymerase-mediated Self-Replicating Label for Protein Quantification; (12) fractional separation and parallel sampling of a single cell's content; (13) the flow manipulator; (14) array of reactor-coated microsticks; (15) array of reactor-coated microcantilevers; (16) array of reactor-coated microtextures; (17) array of reactor-coated microbranches; (18) object-signaling microspheres; (19) object-capturing microspheres; (20) the 3-dimensional compacted microstick; (21) the spacers-in-microcolumn; (22) the electronic binding reliever; (23) the single-round DNA sequencer; (24) the CMOS circuits; (25) demonstration of single cells in microfluidic environments; (26) demonstration of single molecules in microfluidic environments; (27) The Artificial Nerve Terminals; (28) setup of signal measurement & configuration of light sources; (29) miniaturized laser setup; (30) the piezoelectric metrics-based energy reservoir; (31) miniaturized organic light emitting diodes & array of signal flashers; (32) the fiber optic data link; (33) the neural-network combinatorial code board; (34) the parallel signal processor; (35) Customized materials used for microfabrication.

1. The System Architecture. Microsystems are designed in purpose to be able to capture electronic, optical, chemical, biochemical, or human attribute data; mine captured data using algorithms; execute high-speed data transfer through an infrastructure network, internet or wireless interface; and trigger a predefined chemical reaction, biological procedure, mechanical motion or human response. The device combines the four principal components (Signal Acquisition, Signal Interpretation, Signal Representation and Signal Execution) of Pharmacom's 4-D architecture into an operational multiplex system. It allows all procedures (including sample capture, cellular preparation, molecule separation, target selection, etc.) to be carried out within a few minutes.

2. Living Object-Based and Surface Molecule-Mediated (LOSM) Platform

The optimal performance of a biodetection system in its speed, sensitivity and selectivity can be achieved only through a system design based on a maximum simulation of the “natural” situation of molecule-molecule interaction; and a maximum imitation of the natural response mechanism, natural molecule complimentary, natural messaging flow, and natural signaling pathway when a target approaches to and interacts with its potential receiver at a molecular, cellular or organic level. Bioagent detection and identification of the system is accomplished through the LOSM platform and not by artificially extracting living objects and forcefully changing their natural attributes. Living Object-based and Surface Molecule-mediated (LOSM) platform is particularly designed to capture and detect bioagents at single molecule/single bioentity level.

3. The “Living” Reactors

The reactors are the one of most critique components in the Microsystems. We are referred are not simply limited to a group of antibodies or ligands, or those molecules that can bind specifically to the target without displaying significant nonspecific binding with other solution molecules.

A “reactor” having affinity for a target molecule is covalently attached to an insoluble support and functions as bait for capturing the target from complex solutions. The reactors we designed for affinity separations include small organic compounds that are able to dock into binding sites on proteins, inorganic metals that form coordination complexes with certain amino acids in proteins, hydrophobic molecules that can bind nonpolar pockets in biomolecules, proteins with specific binding regions that are able to interact with other proteins, and antibodies, which can be designed to target any biomolecule through their antigen binding sites.

The reactors are fabricated based on (1) the origin of the cells or bacteria and individuality of the substrains in which narrows down targets; (2) the structural uniqueness of the biomarkers in which designs high sensitivity reactors; (3) the surface molecules of the cells or bacteria in which optimizes the binding conditions of reactors; and (4) the cell or bacterium-produced proteins, -released toxins and -induced substrates at metabolic pathways, in which designs high selectivity reactors that enable subtypes distinguish.

4. The “Micromachinery” Reactors

They are artificial objects and designed to act at the situation where a “living” reactor is not available or does not function well.

Tomographic Model. In the tomographic receptor model, the receptor engineer again starts with a known target molecule topography and designs a series of thin planar sections which, when stacked together in the correct order (using positionally-coded docking pins) and bonded, create a solid object containing the desired optimum binding cavity. As in the mosaic model, point charges or dislocations in each planar segment can be used to manipulate cavity features and dimensions to precise tolerances. Unlike the mosaic model, a tomographic receptor can be reconfigured by partial disassembly and replacement of specific planar segments, each of which contributes only locally to the total receptor structure. Hybrid or modular artificial enzymes and two-dimensional sheet like hydrogen-bonded networks are crude analogies in current research.

Imprint Model. Molecular imprinting is an existing technique in which a cocktail of functionalized monomers interacts reversibly with a target molecule using only noncovalent forces. The complex is then cross-linked and polymerized in a casting procedure, leaving behind a polymer with recognition sites complementary to the target molecule in both shape and functionality. Each such site constitutes an induced molecular “memory,” capable of selectively binding the target species. In one experiment involving an amino acid derivative target, one artificial binding site per (3.8 nm)³ polymer block was created, only slightly larger than the (2.7 nm)³ sorting rotor receptors described by Drexler. Chiral separations, enzymatic transition state activity, and high receptor affinities up to K_{d}˜10⁻⁷ have been demonstrated, with specificity against closely competing ligands up to Δ K_{d}˜10⁻² (˜20 zJ). Several difficulties with this approach from a diamondoid engineering perspective include: (1) a sample of the target molecule is required to make each mold; (2) it is currently unknown how to prepare diamondoid castings; and (3) once the imprint has been taken, the site cannot easily be further modified.

Solid Mosaic Model. In the solid mosaic receptor model, the precise shape and charge distribution of the target molecule is already known. Working from this information, a set of diamondoid components could be fabricated which, when fitted together like a Chinese puzzle box, create a solid object having a cavity in the precise shape of the optimum negative image of the target molecule. The mosaic may contain point charges, voids, stressed surfaces, or dislocations to achieve fine positional control. Mosaic components may be as small as individual atoms, so this model is conceptually similar to 3D printing or raster-scan techniques in which the desired cavity formation is constructed atom by atom inside a nanofactory. This model, like the imprint model, cannot easily be reconfigured once it has been constructed because each of the many unique parts may contribute to the entire structure. A protein mosaic model has been designed using cyclic peptides that assemble spontaneously into nanotubes of predefined diameter; incorporation of hydrophobic amino acid side chains on the outside of these tubes leads to spontaneous insertion into bilayers, allowing the tubes to function as transmembrane ion channels. Other examples of mosaic model receptors are mesoporous silica filters with functionalized organic monolayers forming 5.5-nm sieve-like pores, and zeolites and zeolite-like molecular sieves. Zeolites are artificial crystal structures with precise and uniform 0.4-1.5 nm internal void arrays which can also be used as shape-selective catalysts able to favor one product over another that differs in size by as little as 0.03 nm, such as p-xylene and o-xylene. Rational de novo computational design of artificial zeolite templates and crystal engineering has begun.

Pin Cushion Model. The pin cushion receptor is a hemispheroidal or hemiellipsoidal shell through which a number of rods protrude, each of which may be moved radially. When inserted through the shell to varying depths, the endpoints of the rods define a negative image surface which may be made to mirror the topography and charge distribution of a known target molecule. Rods may be tipped with positive, negative or no charge, or they may terminate in any number of functionalized surface segments designed to optimally match parts of the target molecule shape. Other configurations such as a rectangular box, hinged plates with protruding rods, counterrotating rollers, or time-varying rod positioners are readily conceivable. Pin cushion receptors are easily reconfigured to bind different target molecules, hence may be regarded as fully programmable “universal” binding sites. The principal difficulty with the pin cushion receptor is its excessive size (compared to other receptor models) and its greater complexity (since each rod must be controllable individually). Pin cushion receptors can also be used to discover the shapes of unknown molecules: A target molecule is placed in the central cavity with all rods fully retracted, and the rods are slowly slid forward using nanopistons with force reflection feedback, until all pistons register zero force, indicating balance between attractive and repulsive van der Waals interactions, at which point all rod positions are recorded. Rods of differing end tip charge may then be tested for additional attractive potential. The final result is a precise mapping of the target molecule, which data may be stored or transmitted elsewhere for future use.

5. Reactor-Coated Membranes

FT-IR-absorsive films. Special materials (with a 0.1 cm−1 resolution) can be composed and customized for the characterization of explosives in the mid-infrared (7400 cm−1 to 350 cm−1) spectral range. These materials selectively absorb infrared radiation to varying degrees depending upon the chemical nature of the material, producing a vibrational infrared spectrum characteristic of the material. This spectrum provides information on the presence or absence of functional groups and gives an overall characterization (chemical bonding and molecular structure) of the material being examined. Butterfly of FT-IR is a non-destructive analytical technique which can provide both qualitative and quantitative (standards required) information. Unknown samples can be identified either by comparing to the spectra of known substances or to a spectra library in a remote database through wireless communication.

Fluorescent-generating films. The fluorescent film contains dyes with excitation and emission wavelengths that cover the entire spectrum from the near UV to the near infrared. The film with four different surface functional groups can be prepared and that make them compatible with a variety of conjugation strategies. The fluorescent dyes have negligible effect on the surface properties of the polystyrene beads or on their protein adsorption. In order to both decrease nonspecific binding and provide additional functional groups for conjugation, those beads are designed to have a high density of carboxylic acids on their surfaces. Sulfate films are relatively hydrophobic particles that will passively adsorb almost any protein, including albumin, IgG, avidin and streptavidin. Aldehyde-sulfate film, which are sulfate films that have been modified to add surface aldehyde groups, are designed to react with proteins and other amines under very mild conditions. Amine-modified film can be coupled to a wide variety of amine-reactive molecules, including the succinimidyl esters and isothiocyanates of haptens and drugs or the carboxylic acids of proteins, using a water-soluble carbodiimide. The amine surface groups can also be reacted with SPDP (S1531) to yield (after reduction) microspheres with sulfhydryl groups.

Metalloporphyrins films. The role of metalloporphyrins as chemically interactive material in chemical sensors has interested researchers for long time. The rich coordination chemistry of the porphyrin is responsible for their use in chemical sensor applications using the changes induced in their physicochemical properties by the addition of axial ligands. Normally the four nitrogen molecules define a coordination plane, which is called ‘equatorial’. If the metal is coordinatively unsaturated, additional ligands can be linked at the left axial positions. It has also been reported that recently metalloporphyrins have been introduced as coating materials of quartz microbalances to obtain chemical sensors. The main features of such sensor's properties in terms of selectivity and sensitivity are extremely good. The nature of the central metal and the lateral groups are responsible for sensor properties. With only a little variation in synthesis it is possible to obtain sensors with different responses. This feature makes these compounds extremely suitable for sensor applications.

6. Immobilization of the Reactors

Five requirements are considered for determining whether a surface is suitable as a substrate for fabricating reactors. (1) the surface has to be flat on a nanometer scale over a micrometer range, in order to distinguish single proteins from the roughness of the supporting surface. Suitable surfaces in this respect are, for example, mica, graphite, ultraflat gold, and silicon nitride; (2) a strong bond should be formed between the surface and the proteins to avoid that the probing tip removes the proteins; (3) nonspecific adhesion between tip and surface should be minimized; (4) the density of proteins on the surface should be high enough, at least 100 proteins/_m2 to achieve a high frequency of recognition events; (5) in order to distinguish individual proteins, the density should not be too high.

Four approaches are conducted for coupling reactors to substrate: (1) Coupling Affinity Ligands to through Amine Groups. The most common functional target for immobilizing protein molecules is the amine group, which is present on the vast majority of proteins due to the abundance of lysine side chain □-amines and N-terminal □-amines. The immobilization reaction using reductive amination involves the formation of an initial Schiff base between the aldehyde and amine groups, which then is reduced to a secondary amine by the addition of sodium cyanoborohydride; (2) Coupling Affinity Ligands through Carbonyl Groups. Most biological molecules do not contain carbonyl ketones or aldehydes in their native state. However, it might be useful to create such groups on proteins in order to form a site for immobilization that directs covalent coupling away from active centers or binding sites. Glycoconjugates, such as glycoproteins or glycolipids, usually contain sugar residues that have hydroxyls on adjacent carbon atoms, which can be periodate oxidized to create aldehydes. Aldehydes on the carbohydrate portion of glycoconjugates may be used to covalently link with affinity supports through an immobilized hydrazide, hydrazine or amine group by Schiff base formation or reductive amination; (3) Coupling Affinity Ligands through Sulfhydryl Groups. It is often advantageous to immobilize affinity ligands through functional groups other than just amines. In particular, the thiol group can be used to direct coupling reactions away from active centers or binding sites on certain protein molecules. Since amines occur at many positions on a protein's surface, it is usually difficult to predict where a coupling reaction will occur. However, if sulfhydryl groups which typically are present in fewer numbers are targeted for immobilization, then coupling may be done at discrete sites in a protein or peptide. Thiol groups (sulfhydryls) can be indigenous within a protein molecule or they may be added through the reduction of disulfides or through the use of various thiolation reagents. Sulfhydryls also can be added to peptide affinity ligands at the time of peptide synthesis by adding a cysteine residue at one end of the molecule. (4) Coupling Affinity Ligands through Carboxyl Groups. The carboxyl group is a frequent constituent of many biological molecules. Particularly, proteins and peptides typically contain numerous carboxylic acids due to the presence of glutamic acid, aspartic acid and the C-terminal a-carboxylate group. Carboxylic acids may be used to immobilize biological molecules through the use of a carbodiimide-mediated reaction. Although no activated support contains a reactive group that is spontaneously reactive with carboxylates, chromatography supports containing amines (or hydrazides) may be used to form amide bonds with carboxylates. Molecules containing carboxylates may be activated to react with an immobilized amine (or hydrazide) through reaction with the water-soluble carbodiimide EDC. EDC reacts with carboxylates to form an intermediate ester that is reactive with nucleophiles such as primary amines.

Four Techniques are Utilized for Immobilizing Reactors:

Photochemically immobilize reactors onto a fibre-optic silica surface. The approach is based on a photoreactive benzophenone derivative that is bound to SiO₂ surfaces of the optical fibre via a silane anchor. The benzophenone derivative is 4-allyloxybenzophenone, synthesized by standard procedures which will be further used to synthesize the 4-(3′-chlorodimethylsilyl) propyloxybenzophenone and 4-(3′-dichloromethylsilyl) propyloxybenzophenone by regular hydrosilation procedures. After silanization with the benzophenone derivatives, the fibres will be immersed in a cholera toxin B subunit solution and illuminated with UV light (wavelength>345 nm). As a result of the photochemical reaction, a thin layer of the antigen will be covalently bound to the benzophenone-modified surface. As the control, the photochemically modified fibre-optics will be tested as immunosensors in the detection of cholera anti-toxin antibody and gone through chemiluminescence measurements. A secondary antibody labeled with horseradish peroxidase acted as the marker for the cholera toxin antibody. A photo-electronic set-up will be designed specifically to monitor the signal.

Immobilize reactors on to a fiber-optic silica surface by chemical oxidation. The procedure consists in the chemical oxidation of pyrrole-biotin monomers that are readily deposited as a thin film of poly(pyrrole-biotin) polymer on to the end-face of the fiber. The film is designed to be translucent to enable photon coupling within the fiber transducer and its presence is demonstrated by means of fluorescent micrographs of bound rhodamine-labeled avidin. Fiber-optics modified with cholera toxin B subunit molecules is tested for sensitivity, non-specificity, and overall practicality. As expected, the fiber-optic immunoassay for the detection of anti-cholera toxin antibody should be up to three orders of magnitude more sensitive than the classical enzyme-linked immunosorbent assay (ELISA).

Layer-by-layer electrostatic self-assembly. The LBL/ESA will be used for developing the sandwiched microsticks. LBL/ESA utilizes the electrostatic interactions between molecules to assemble thin films one monolayer at a time. LBL/ESA permits precise control of the optical characteristics of the sensing surface nanostructure, and produces ordered immunoglobulin monolayers with optimal packing density for analyte binding. Analyte binding alters the optical properties of the attached thin film, immediately modifying the transmission and reflection characteristics of the fiber. This produces an observable output that indicates the presence and concentration of a given target analyte. In this particular project, different types of “reactors” that bind to corresponding targets in distinctive format will be immobilized on the thin film that is attached to the optical fiber.

Biotinylation. To minimize the overwhelming shortcomings such as unstable peptide/protein attachment and “orientation and effective interaction” problem in the immobilization, a strategy that site-specifically immobilize peptides on a glass plate using avidin-biotin interaction will be adopted. In our procedure, N-terminally biotinylated receptor proteins are immobilized onto an avidin-coated substrate using a conventional microarray spotter. Avidin is an extremely stable protein, making it an excellent candidate for slide derivatization and immobilization. Each avidin/streptavidin molecule can bind rapidly and almost irreversibly up to four molecules of biotin. Avidin also acts as a molecular layer that minimizes non-specific binding of proteins to the surface, thereby eliminating blocking procedures and minimizing background signals in downstream screenings. The some of reactor proteins can be site-specifically biotinylated at their C-terminal end using an intein-mediated expression system. The expressed C-terminal fusion protein can be biotinylated in a single step on a suitable substrate. This highly robust novel protein array features uniformly oriented proteins, which ensures all immobilized proteins to retain their full biological activities. The use of biotin-avidin interaction for immobilization also allows the proteins to withstand even the most harsh conditions used for downstream screenings.

(5) Stabilize hydrogen-bonded poly(N-isopropylacrylamide) multilayers using a dual electrostatic/hydrogen bonding copolymer. Multilayer thin films were prepared based on hydrogen bonding between poly(N-isopropylacrylamide) (PNiPAAm), and poly(styrene sulfonate-co-maleic acid) (PSSMA). Intercalated PAH layers were included to improve the pH stability of the film by introducing electrostatic linkages into the assembly. Film construction was studied as a function of pH of the deposition solution and the number of inserted PAH layers. Film morphology varied significantly with incorporation of PAH into the film. By intercalating several PAH layers within the PNiPAAm/PSSMA assembly, the pH stability of the films at pH 5.8 has also been substantially improved.

7. Kinetic Tuning of the Microenvironment for Reactor-Target Interaction

Selectivity is determined by the nature of the receptor preparation, the type of target molecule used in the assay and the binding properties of the analyte being assayed. Reactor preparations containing a single reactor or a heterogeneous population of binding sites can be used. If a heterogeneous receptor population is used, it is important to choose the target molecule with care. If the target molecule and the analyte bind to more than one class of reactor, depletion of both target molecule and analyte might occur when using heterogeneous reactor.

Kinetics of the binding. The relationship between a fixed amount of reactor and target molecule and the formed complex can be estimated with the equation, where [R], [L*] and [RL*] are the concentrations of reactor, target molecule and complex, respectively, and k₋₁ and k₁ are the dissociation and association rate constants in the equation (2), respectively:
[R]+[L*][RL*]
Since the amount of reactor is limited, saturation of the binding sites will occur at high concentrations of target molecule. The figure demonstrates a typical saturation curve for reactor-target binding. The total amount of binding sites (Bmax) is found on the ordinate at the point where the curve reaches its plateau. The amount of target molecule that gives a 50% saturation of the reactor represents the dissociation constant K_{d}.
Bmax and Kd can also be calculated using the equation (3):

$$[ {RL}^{*} ] = \frac{[ {L}^{*} ] * {B}_{max}}{[ {L}^{*} ] + {K}_{d}}$$

The dissociation constant K_{d} is inversely proportional to the affinity of the target molecule for the reactor and is defined by the ratio of the dissociation and association rate constants in the equation (4):

$${K}_{d} = \frac{{K}_{1}}{{K}_{2}}$$

Addition of a competitive analyte will displace a certain amount of the target molecule, depending on the concentration of the former and on its equilibrium binding constant K_{d}, resulting in two types of receptor complexes, as described in equation (1). By varying the amount of analyte and keeping the concentration of target molecule and reactor constant, calibration curves can be constructed. The IC₅₀ value, i.e. the amount of analyte displacing 50% of the bound target molecule can be determined from these curves. The affinity constant of the analyte (Kᵢ) is related to the IC₅₀ as described by the Cheng-Prusoff equation (5):

$${IC}_{50} = {K}_{i} * ( 1 + \frac{[ {L}^{*} ]}{{K}_{d}} )$$

Measure the sensitivity of reactor-target assays. The sensitivity of specific binding of a target molecule to its reactor depends on the ratio of concentration/K_{d} of the target molecule being assayed. An important parameter is the limit of detection (LOD), which can be defined as the minimum concentration of analyte at which the fraction of bound target molecule is significantly smaller than the fraction of bound target molecule in the absence of analyte, and can be calculated using the equation (6):

$$LOD = {γ}_{1} [ 1 + \frac{{K}_{n}^{*} * {K}_{d}^{*}}{{[ R ]}_{0}} ( 1 + \frac{[ {L}^{*} ]}{{K}_{d}^{*}} ] ( 1 + \frac{[ {L}^{*} ]}{{K}_{d}^{*}} ) {K}_{d}$$

where:
γ₁=a parameter from Student t-test which characterizes the error of the determination of the concentration of analyte in the absence of analyte
Kₙ*=the constant for non-specific binding of the target molecule
K_{d}*=the dissociation constant of the analyte
[R]₀=the total reactor concentration
[L*]=the concentration of the free target molecule
K_{d}=the dissociation constant of the analyte
It can be seen from equation (6) that several factors determine the sensitivity of a receptor assay.
(a) The LOD is directly proportional to the K_{d} of the analyte. In other words, when the analyte has a high affinity (low K_{d}), less of it can be detected. (b) Since the LOD is also directly proportional to the concentration of target molecule, a minimum of the target molecule should be used to increase sensitivity. A free concentration of target molecule equal or close to the dissociation constant is considered to be a good compromise. (c) The lower the amount of non-specific binding, the higher the sensitivity, resulting in a decrease in the LOD.
Measure the selectivity of reactor-target assays. During a receptor assay only pharmacologically active compounds will bind to its reactor, while inactive compounds belonging to the same structural class or compounds belonging to another structural class will not bind. Selectivity is determined by the nature of the receptor preparation, the type of target molecule used in the assay and the binding properties of the analyte being assayed. Reactor preparations containing a single reactor or a heterogeneous population of binding sites can be used. If a heterogeneous receptor population is used, it is important to choose the target molecule with care. If the target molecule and the analyte bind to more than one class of reactor, depletion of both target molecule and analyte might occur when using heterogeneous reactor.
8. Dynamic Sample Collection Interfaces

The sample collection module also include: 1) a mini-syringe for scaled collection of liquid; 2) a mini-pressure hose for volumetric breath of air; 3) a mini-screw for penetration of solid matter; 4) a pin-tip for scratch of object surface; 5) extendable pipes that are jointed with the sampling interfaces described as above for extending to different locations within a certain range; 6) preconcentrator: for concentrate particles of interest from a small volume of air; and 7) volumetric container: for concentrate agents of interest from a small volume of liquid.

Liquid phase sampling: It is a form of liquid chromatography to separate compounds that are dissolved in solution. It consists of a reservoir of mobile phase, a pump, an injector and a separation column. The molecules of sample are separated by autonomously injecting a plug of the sample mixture onto the column. The different components in the mixture pass through the column at different rates due to differences in their partitioning behavior between the mobile liquid phase and the stationary phase.

Solid phase sampling: It is a unique solid phase extraction cartridge (SPE) designed for repeated extractions of drugs from complex matrices, such as plasma, serum, supernatants of cell cultures and fermentation broth. It is based on polymeric particles with a hydrophobic internal surface and a biocompatible external surface. The biocompatibility can be obtained by attachment of the plasma protein □1-acid glycoprotein (AGP) on the external surface of the particles. Immobilized AGP is an extremely stable protein, which tolerates all the organic solvents used in off-line solid phase extractions (SPE). The pores of the particles can be designed small enough to exclude the plasma proteins and other macromolecular compounds, whereas particle molecules and other low-molecular-mass compounds can penetrate the pores and be adsorbed to the hydrophobic inner surface. Since the cartridge is polymer based, it can be used between pH 2-13. This property will give possibilities of extracting ionized analytes in their uncharged form. The uncharged analyte has higher affinity to the hydrophobic inner surface of the cartridge, it gives an improved recovery.

Aerosol phase sampling: Collecting aerosol samples demonstrates the greatest challenge among all types of sampling situation that we may encounter. The efficiency of the aerosol collector depends on the size of the aerosol agent, the type of filter, the velocity of the air, and the type of microbe. Four different collection mechanisms govern particulate air filter performance: inertial impaction, interception, diffusion, and electrostatic attraction. The first three mechanisms are the most important for mechanical filters and are influenced by particle size. Impaction occurs when a particle in an air stream passing around a filter fiber, because of its inertia, deviates from the air stream and collides with a fiber. Interception occurs when a particle in the air stream passing around filter fibers comes in contact with a fiber because of its size. Impaction and interception are dominant for large particles (>0.2 microns). Diffusion occurs when the random (Brownian) motion of a particle causes that particle to contact a fiber. Diffusion is the dominant collection mechanism for smaller particles (<0.2 microns). The combined effect of these three collection mechanisms results in the classic collection efficiency curve. The fourth mechanism, electrostatic attraction, plays a minor role in mechanical filtration because, after fiber contact is made, small particles are retained on the fibers by a weak electrostatic force.

9. Autonomous Reagent Supplier

It is designed to contain injectors, fluid droplets and electronic triggers. Injectors are attached directly to adjacent reservoirs containing reagents. Droplets are 20-100 μm in diameter and have volumes in the 0.1 to 1 nL range. Some of those on-chip reservoirs are designed to have 15 μL volumes thus they are able to provide up to 10⁵ reagent droplets, enough for 1 assay per minute for 90 days.

10. Protein Extraction without Centrifugation

The sample is collected and analyzed in a manual of point-of-care at real time, several customized reagent kits are used for the protein extraction from the target cells. The reagent is a 10×-concentrated mixture of specialized detergents and buffer that enables gentle extraction and purification of target proteins directly from culture media without cell harvest, mechanical disruption, or extract clarification. Recombinant proteins can be directly screened in the crude extract or purified by adding an affinity matrix, washing the matrix-target protein complex, and eluting the purified protein from the matrix. The ability to perform the entire procedure in the original culture tube or multi-well plate leads to increased convenience and speed when processing multiple samples. By using the reagent, experimenters no need to separate cells from culture media, no need to mechanically disrupt cells, and no need to clarify extracts prior to purification since it is compatible with popular purification methods such as IMAC, GST, immunoaffinity.

11. T7RNA polymerase-Mediated Self-Replicating Label for Protein Quantification

T7RP and SRLPQ are merged to be a single-cell based proteomic profiling technology and integrated together to fit into a microarray platform. The three-step assay includes coating of the wells and immunocomplex formation; reaction with the SA-T7RP complex and finally self-replication of T7RP; and luciferase synthesis.

The proteins present at low concentrations are usually the ones that mediate the cellular response to various stimuli and might be the ones that involves in the early-stage disease development. The direct quantitative analysis of the target proteins provides more accurate information about cellular systems. The comparison of protein expression profiles in patients and normal samples (differential profiling) can reveal potential biomarkers for diagnosis, prognosis and monitoring of disease progression at early stages.

T7 RNA polymerase (T7RP), which has the unique ability to self-replicate in vitro and catalyze the in vitro synthesis of a second enzyme (firefly luciferase), is cloned and amplified in E. coli. Biotinylation of T7 RNA polymerase (BT7RP) complexed with streptavidin (SA) is added to the immunocomplex. The bound T7RP is determined by in vitro coupled transcription/translation.

Two approaches are explored. T7RP-DNA, placed downstream of the T7 promoter, served as the template for the first reaction (self-replication). Then, T7RP is transferred to the second expression reaction in which the Luc-DNA served as template (detection. (a) T7RP acts on firefly Luc-DNA, located downstream of the T7 promoter, to produce several molecules of active luciferase which is measured by its characteristic bioluminogenic reaction. (b) T7RP acts on T7RP cDNA (T7RP-DNA), positioned downstream of the T7 promoter, to generate several T7RP molecules (self-replication phase) which, in turn, act on Luc-DNA to produce luciferase (detection phase. The resulting signal amplification is due to the generation of many enzyme molecules in solution.

The coupled transcription/translation process consists of a series of complex reactions that require the concerted action of numerous factors, such as RNA polymerase, ribosomal subunits, translation initiation, elongation and termination factors, aminoacyl-tRNA synthetases, etc. The final outcome is a simple linear relationship between input T7RP and the in vitro synthesized protein over a wide range of T7RP concentrations. This forms the basis for the development of a T7RP-based signal amplification system exploiting T7RP as a label.

The experiment of coupling T7 RNA polymerase with in vitro transcription and translation establishes a relationship between the input T7RP and the synthesized protein in an in vitro transcription/translation system. The expression of firefly luciferase is chosen because this enzyme can be detected with high sensitivity by using its characteristic bioluminogenic reaction. The coupled transcription/translation process consists of a series of complex reactions that require the concerted action of numerous factors, such as RNA polymerase, ribosomal subunits, translation initiation, elongation and termination factors, aminoacyl-tRNA synthetases, etc.

The modified technology offers a number of significant advantages. (i) The assay does not use radioactive isotopes. (ii) In contrast to IDAT, which requires tedious denaturing gel electrophoresis and autoradiography, the present assay is performed entirely in microtiter wells, thereby allowing for automation and high-throughput analysis. (iii) A quantitative relationship is established between the luminescence signal and the amount of antigen with a dynamic range covering almost four orders of magnitude. (iv) Compared to IDAT, the proposed assay is much shorter. Indeed, after immunocomplex formation, IDAT requires a 4 h transcription step followed by time-consuming electrophoresis and autoradiography. In contrast, the present technique requires much less time for quantification of the immunocomplexes. (v) Because of the self-replication reaction, amplification in the proposed system is exponential, whereas IDAT involves linear amplification of the label. Suitable enhancer and transcription termination sequences can be incorporated to increase the yield of both self-replication of T7RP and luciferase synthesis. (vi) Insertion of both T7RP-DNA and Luc-DNA templates, under the control of the T7 promoter, into a single vector can be used for even higher yields. Besides firefly luciferase DNA, cDNAs for other highly detectable proteins can be employed, e.g. green fluorescent protein, alkaline phosphatase, aequorin, etc.

12. Fractional Separation and Parallel Sampling of a Single Cell's Content

The strategy enables observing functional related cellular entities and metabolic related molecules at a fashion of section plane. The procedures includes:

(i) a drop or pierce of sample is collected from a single resource;

(ii) the sample is divided as five portions;

(iii) fractional process of the sample is proceeding within a unified sample processor that jointed with separated chambers;

(iv) general procedures that process the sample in different modes involve (1) adjusting a liquid's volume in a segment; (2) releasing a reagent to the adjacent segment; mixing reagents and samples; (3) agitating and incubating a reaction mixture under a given thermal condition; and (5) washing and removing waste from a segment;

(v) the sample from single resource is divided as five classes ranged from the biggest entity to the smallest entity. The divided or “pre-classified” samples are pumped into five different chambers:

(1) Chamber-in-Mode-1: different cells from different sampling points are routed to different reaction-chambers.

(2) Chamber-in-Mode-2: same cells from a single sampling point are routed individually as a single cell to different reaction-chambers.

(3) Chamber-in-Mode-3: fractions of a single cell are routed to different reaction-chambers.

(4) Chamber-in-Mode-4: different molecules from a single cell are routed to different reaction-chambers.

(5) Chamber-in-Mode-5: same molecules from a single cell are routed to different reaction-chambers

13. Flow Manipulator

The microcomponents are integrated and optimized based on computational simulations. The simulation on flow in micro channels and capillaries; fluid-structure-electrostatics; fluid-structure interaction in a passive microvalve, and microfluidic oscillator; and other fashions of flow movements are conducted in order to determine a simulation capability appropriate for fluid flow through smooth and textured channels, and optimum flow or mixing characteristics.

The carrier inlets and sample inlets is generated based on laminar behaviors. Since sample flow is controlled by the flow ratio of the right and left carriers and a multi outlet flow switch is implemented. The samples flow along the top and bottom wall of the channel where the flow rate is normally small, and a miniaturized sample transfer system realizing perfect sheath flow is designed for flow cytometers.

Use the unique capabilities of 3D excimer laser ablation, to simultaneously create textures and features within the flow channels. The coaxial sheath flow in the cylindrical channel is designed since the flow distribution of the sample is critically changed with the pressure and the flow rate of the carrier, a 3-D finite element fluidic analysis is indispensable to design the microflow cell. To realize the vertical sheath flow with simple inlet structure of carrier and sample, the strategy of the two steps introduction of carrier flows will be adopted.

Use injection molding or embossing techniques for capping the devices without blocking or restricting microfluidic channels. Connect the patterned devices to other components of the system in a way which ensures minimal dead-volume, no leakage and no defects within channel and reservoirs.

14. Array of Reactor-Coated Microsticks

A microstick basically is an optic fiber. It is coated with a thin film-membrane and appropriate reactors are stabilized on the surface. It is designed to carry excitation light produced by the miniature light-emitting diodes (LED) through interior of the optic fiber. An event of molecule interaction occurred at the surface of the optic fiber re-features the original patterns of the wavelength. The optical signals can be interrogated and collected at multiple wavelengths with different signal intensities, different phases, polarization and exited state lifetimes. The signals are directed to another end of the optical fiber where the variance of wavelength can be detected immediately or the energy can be further utilized by OLED.

(1) The Long Period Grating (LPG) technology is used for the fabrication of the microstick. It is a spectral loss element that scatters light out of an optical fiber at a particular wavelength based on grating period, fiber refractive index, and the refractive index of the surrounding environment. The microstick with specially designed affinity coatings or swellable polymers will cause selective, quantitative changes in the refractive index ‘seen’ by the LPG in the presence of target molecules. The epitope-containing reactor is immobilized on the surface of a planar waveguide. The sample containing the target molecules is flowed over the surface. Some of them bind to the receptors. Laser light is directed through the waveguide within the optical fiber using total internal reflection. An evanescent wave extends a short distance outside of the waveguide. As the coating absorbs target molecules the refractive index changes, causing a shift in the wavelength of the scattered light. This wavelength change is demodulated to (1) excite the florescence material applied in a signal amplification well for an immediate signal flashing; (2) activate the organic light emitting diodes which serve as an array of signal flashers; or (3) trigger an event of the light-to-electric conversion for a remote data transmission through the fiber optical data link.

(2) The evanescent waveguide is made of a 200 nm thick silicon nitride (Si3N4) core layer, sandwiched between two 1.5 μm thick silicon dioxide (SiO2) layers. The refractive indices are typically n1^{˜}1.46 and n2^{˜2} for SiO2 and Si3N4 layers, respectively so that the difference in the refractive indices of the core and cladding is large. The advantage of this sensor lies in the fact that it can be used directly in liquid environments to possess a high degree of selectivity and sensitivity, by exploiting multiple reflection technique of light in silicon dioxide (SiO2)/silicon nitride (Si3N4) waveguide structure as the optical transducer of the sensor.

(3) Any bioagent binds to the sensory layer affects the evanescent wave of the light propagating in the fiber. The effect is primarily a result of (1) a change in the index of refraction to which the evanescent wave is subject and (2) increased scattering of light. The evanescent wave is shallow enough that the microstick exhibits a significant response to the bioagent. The optical signals can be interrogated and collected at multiple wavelengths with different signal intensities, different phases, polarization and exited state lifetimes. The signals are directed to another end of the optical fiber where the variance of wavelength can be detected immediately or the energy can be further utilized by OLED.

(4) Functions of the reactor-coated microstick (RCM) include: (1) has an optical fiber carries excitation light produced by a miniature LED to the thin-film coating at the end of the optical fiber; (2) measures samples both gas and liquid phases; (3) is immune to environmental changes in pH, salinity and ionic strength.

RCM is immune to interference from moisture, carbon dioxide, methane and other substances; (4) has fast response time—between 0.01-1 second for aerosol samples and between 30-120 seconds for air or liquid samples; (5) has a long life—more than 1 year; (6) allows a continuous contact with the sample; (7) no needs frequent calibrations; and (8) sensing temperature range is −30° C. to +50° C.

(5) Parameters of the microstick: (1) Transmission Distance: System Complexity Increases with Transmission Distance; (2) Types of Optical Fiber: Single-mode or Multimode; (3) Dispersion: Incorporate Signal Regenerators or Dispersion Compensation; (4) Fiber Nonlinearities: Fiber Characteristics, Wavelengths, and Transmitter Power; (5) Operating Wavelength: 780, 850, 1310, 1550, and 1625 nm Typical; (6) Transmitter Power: Typically Expressed in dBm; (7) Source Type: LED or Laser; (8) Receiver Sensitivity/Overload Characteristics: Typically Expressed in dBm; (9) Detector Type: PIN Diode, APD, or IDP; (10) Modulation Code: Digital; (11) Bit Error Rate (Digital Systems Only): 10⁻⁹, 10⁻¹² Typical; (12) Signal-to-Noise Ratio: Specified in Decibels (dB); (13) Number of Connectors or Splices in the System: Signal Loss Increases with the Number of Connectors or Splices; (14) Environmental Requirements and Limitations: Humidity, Temperature, Exposure for Sunlight; (15) Mechanical Requirements: Flammability, Indoor/Outdoor Application; (16) Coating process: (a) in situ & (b) monomer vapor phase deposition to control the surface morphology of the polymer; (17) Substrate: (a) hydrophilic & (b) hydrophobic for polymer morphology control; (18) Metallic Substitution: Incorporation of Cu2+ ions in polypyrrole structure is expected to provide selective and reversible binding sites for organophosphates; (19) Waveguide: Influence of sensing element length and diameter; (20) Light Intensity: Influence of light intensity through fiber; (21) Sensitivity: Influence of concentration of DMMP; (22) Selectivity: Sensor response to other gases; (23) Reversibility: Sensor reversibility in terms of presence and absence of DMMP; (24) Thermal stability: Sensor response due to temperature variations; (25) Durability (aging): Influence of aging on sensor device.

15. Array of Reactor-Coated Microcantilevers

Electron beam lithography (EBL) based microcantilevers are designed for facilitating simultaneous analysis of multiple samples for multiple analytes. Create arrays of silicon cantilever from 6 to 10 micrometers long, half a micrometer wide, and about 150 nanometers thick, with a one-micrometer square at the end. The cantilever is coated with unique reactors and the paddle arrays then can be bathed in a solution containing the targets to adhere to the reactors. A large array of paddles are mounted on a piezoelectric crystal that can be made to vibrate at frequencies on the order of 5 to 10 megaHertz (mHz). A single one of these cantilevers weighs about 1.2 picograms, and vibrates at frequencies in the neighborhood of 10 megaHertz. Adding just a few objects to a cantilever would be enough to change its resonant frequency (about 10 kHz). The minimum detectable mass for a living object can be measured at the level of attogram, and arrays of cantilevers coated with various reactors could allow testing for a wide variety of bioagents at the same time.

16. Array of Reactor-Coated Microtextures

They are designed based on the modifications of the technologies that are used for fabricating microsticks and microcantilevers. Arrays of reactor-coated reactors are immobilized at surface of a microtexture; the supporting bond connects the object-capturing molecule to polymer-made substrate and the substrate is electronically wired; sample flows through, specific target agent interacts with the reactor and thus the microtexture is “loaded”; and the event of binding or interaction triggers a specific optic/electronic signal. A group of microtextures, which conduct distinctive molecule-molecule interaction and reflect distinctive signal respectively, are remotely coupled with the Fiber Optic Data Link at a control monitoring system which can be handheld or in house.

17. Array of Reactor-Coated Microbranches

They are designed based on the modifications of the technologies that are used for fabricating microsticks and microcantilevers. The metrics used for the microbranches and the CMOS format integrated into the microbranches has little differences in comparison with the microtextures.

18. Object-Signaling Microspheres

The microsphere are designed to have: 1) unique refractive index and density; 2) large specific surface area; 3) improved binding kinetics over planar surfaces; 4) robust statistics; 5) low autofluorescence; 6) low nonspecific binding; 7) hydrophilicity; and 8) easy manipulation.

Proteins, oligonucleotides, polysaccharides, lipids, or small peptides can be adsorbed or chemically coupled to the surface of microspheres to capture analytes that are subsequently measured by a fluorochrome-conjugated detection molecule.

Generally used for reactors' attachment to substrate of microsphere include easily reactive components such as primary amines, sulfhydryls, aldehydes, carboxylic acids, hydroxyls, phenolic groups and histidinyl residues. The silica substrate first is activated with a compound that is reactive toward one or more of these functional groups. The activated complex then can generate a covalent linkage between the reactor and the support, resulting in molecule immobilization. The targeted analytes which are negatively charged biomolecules, bind to the coated microspheres in the presence of divalent cations (e.g. Ca2+, Mg2+). The microspheres can attach to the interior spacer inside a microcolumn depending on a built-in electrostatic spot.

Each of molecules or living objects can be distinguished and carried away by a correspondent microsphere which is labeled with fluorescent signals. These signals are derived from a specific biomarker such as a unique antigen attached to an antibody that is labeled with a multicolored fluorescent signal.

The fluorescent microspheres contain dyes with excitation and emission wavelengths that cover the entire spectrum from the near UV to the near infrared. Because long-wavelength (>680 nm) light can penetrate solid matter such as tissues and glasses, the far-red- and infrared-fluorescent microspheres allows conducting tests in the microsystem that were not previously possible with beads that emit at shorter wavelengths.

Type-1 fluorescent microspheres, the blue-fluorescent beads with excitation/emission maxima of 350/440 nm contain an improved blue-fluorescent dye that provides superior brightness and a longer shelf life.

Type-2 fluorescent microspheres, the yellow-green-fluorescent beads are excited very efficiently using the 488 nm spectral line of the argon-ion laser and have exceptionally intense fluorescence.

Type-3 fluorescent microspheres, the orange-, red-orange- and red-fluorescent beads have excitation maxima of 540 nm, 565 nm and 580 nm, respectively.

Type-4 fluorescent microspheres, the nile red-fluorescent beads have broad excitation and emission bandwidths, making them compatible with filter sets appropriate for fluorescein, rhodamine and Texas Red dyes.

Type-5 fluorescent microspheres, the crimson- and dark-red-fluorescent beads are efficiently excited by the 633 nm spectral line of the He—Ne laser. Although the dark-red-fluorescent beads are significantly less fluorescent than the crimson-fluorescent particles, they fluoresce at wavelengths that are longer than, and clearly distinguishable from, those of the crimson-fluorescent particles.

Type-6 fluorescent microspheres, the far-red-fluorescent beads with excitation/emission maxima of 690/720 nm are compatible with diode lasers-inexpensive excitation sources that are increasingly being used in fluorescence instrumentation. These far-red-fluorescent beads may also prove useful for making direct fluorescence measurements in auto-fluorescent materials such as blood, plant tissues and marine organisms.

Type-7 fluorescent microspheres, the infrared-fluorescent beads with excitation/emission maxima of 715/755 nm are the longest-wavelength fluorescent microspheres currently available from any source. These beads absorb and emit at wavelengths at which most tissues are almost optically transparent.

Type-8 fluorescent microspheres, the europium luminescent and platinum luminescent beads have excitation/emission maxima of 340-370/610 nm and ˜390/650 nm, respectively, and decay times of >40 microseconds for the platinum microspheres and >600 microseconds for the europium microspheres, far longer than that of conventional fluorescent probes and autofluorescent samples. The beads can be useful as standards for time-resolved microscopy and for tracing applications in highly auto-fluorescent samples.

The fluorescent microspheres are designed to have in a variety of sizes. The smallest microspheres are currently about 0.02 μm in diameter, with a coefficient of variation (CV) of about 20%, as determined by electron microscopy.

The beads with four different surface functional groups are prepared and that make them compatible with a variety of conjugation strategies. The fluorescent dyes have negligible effect on the surface properties of the polystyrene beads or on their protein adsorption. In order to both decrease nonspecific binding and provide additional functional groups for conjugation, those beads are designed to have a high density of carboxylic acids on their surfaces.

Sulfate beads are relatively hydrophobic particles that will passively adsorb almost any protein, including albumin, IgG, avidin and streptavidin.

Aldehyde-sulfate beads, which are sulfate microspheres that have been modified to add surface aldehyde groups, are designed to react with proteins and other amines under very mild conditions.

Amine-modified beads can be coupled to a wide variety of amine-reactive molecules, including the succinimidyl esters and isothiocyanates of haptens and drugs or the carboxylic acids of proteins, using a water-soluble carbodiimide. The amine surface groups can also be reacted with SPDP (S1531) to yield (after reduction) microspheres with sulfhydryl groups.

The yellow-green-fluorescent microspheres are conjugated to biotin and streptavidin, and yellow-green-fluorescent, red-fluorescent, europium luminescent, platinum luminescent and nonfluorescent microspheres are conjugated to NeutrAvidin biotin-binding protein. These microsphere conjugates will provide us with valuable tools for improving the sensitivity of flow cytometry applications and immunodiagnostic assays.

19. Object-Capturing Microspheres

Each bead set can be coated with a reagent specific to a particular bioassay, allowing the capture and detection of specific analytes from a sample. Lasers excite the internal dyes that identify each microsphere particle, and also any reporter dye captured during the assay. Our method allows multiplexing of up to 100 unique assays within a single sample, both rapidly and precisely.

For example, some of toxic substances can be designed to be bound via carboxy groups on the bead's surface using proven carbodiimide coupling chemistry: (1) Surface chemistry: Carboxyl groups; (2) Binds: Primary amine groups (after activation using EDC and NHS); (3) No. of COOH groups per bead: ˜1×10⁸; (4) Form: Stabilized stock suspension; (5) No. of assays: 1 ml is sufficient for 500 assay points.

The assays involve the interaction of immobilized, bead-bound capture molecules with a reaction partner (analyte) in solution. A reporter molecule, specific for the analyte, can be used to quantify the interaction. Each reaction (bead set) can be identified by its spectral signature after irradiation by the red classification laser. The attendant reporter signal from each reaction can be simultaneously quantified by fluorescence generated by the green reporter laser.

20. The 3-Dimensional Compacted Microarray (3-DCM)

The various types of 3-dimensional compacted microarray (3-DCM) are implemented in the microsystem. (1) The arrays of reactor-coated microsticks are fabricated to have the potential to offer high-throughput detection of proteins, DNA, RNA, peptides and the entire cell respectively. (2) Each group of the microsticks are coated with correspondent reactors, and “hosted” by each of unique microchambers that is designated to have a suitable micro-environment for an optimal molecule-molecule interaction. The self-contained microentity is called “microstick-in-column”. (3) The microstick-in-columns can be resided in reagent-contented air or a reagent-contented liquid. (4) The temperature outside or inside the column can be unjustified by thermal sensors, rapid thermal generators and system controlling software. (5) Some of microstick-in-columns perform their electrostatic activity by joining with microelectrodes at two ends; some perform light transmission by linking to a miniature LED at terminal and a miniature OLED at another terminal. (6) Multiple groups of the microstick-in-column are orchestrated by the a number of integrated algorithms, the Neural-Network combinatorial code board and the parallel signal processor.

21. The Interspacers-in-Microcolumn

The interior spacer is an electrostatic device that is designed to temporally hold object-carrying microspheres when they entered the Sample Release Chamber. The microspheres will be released through a subtle electrostatic change after the object molecules they are carrying are being eluted and washed away. Steps of the microchromatography: 1) Samples from the Sample Fluidifier entered into an pre-equilibrated affinity chromatography microcolumn; 2) the interior spacer temporally holds object-carrying microspheres based on electrostatic force; 3) the attached microspheres remains attachment with the interior spacer but the objects which microspheres were carrying are eluted by changing pH value and organic solvent concentration; 4) the unbound microspheres are released from the microcolumn by adjusting electrostatic strength.

22. The Electrostatic Binding Reliever

Autonomously disassociate the objects which were attaching to the receptors at microsticks. It allows new target to be approached and bound from next wave of sample flow.

1) The microdevice is individually but coupled with a sensing element in a close-by environment. The microdevice can autonomously disassociate a captured object that attaches to a reactor at surface of a sensing element based on an electrostatic mechanism. The action leaves a space for a new target to approach and bind to the sensing element as the next wave of sample flows in.

2) The microdevice is implemented as a part of the sensing element itself, which is able to autonomously disassociate an object that binds to a reactor at surface of the sensing element as the electrostatic stage varies.

23. The Single-Round DNA Sequencer. It is designed to sequence single DNA molecules in a microfluidic environment or microchannel network. In the microsystem: (i) the liquid containing DNA or RNA is pumped by electric fields from Chamber-I where raw sample has been digested to Chamber-II where the enzymes cut the DNA or RNA into segments of different lengths; (ii) The DNA or RNA fragments of various sizes can be sorted by using two different methods. The 1^{st} method: Chamber-III is implemented with fibrous strands of a polymer in liquid. Movement of the DNA or RNA is retarded by the polymer strands. Small fragments of DNA or RNA move through the web of polymer strands faster than the larger ones, resulting in separation. The 2^{nd} method: An electrical field is applied briefly to DNA of varying lengths with grooves in the base. The grooves, which confine DNA, create an unfavorable energetic climate. The larger DNA molecules, in essence, become claustrophobic, and when the energy field is removed, the molecules recoil, or push out of the groove. The smaller pieces remain trapped, and the electrical field is applied again. By the end, the largest pieces of DNA, the ones that keep recoiling out of the grooves, can be isolated from the smaller pieces that remain trapped. (iii) The selected DNA or RNA segments are then pumped to Chamber-IV where they are tagged with fluorescent dyes for subsequent single run sequencing. The chemical mixture for determining the sequence, which contains primers, enzymes, buffers, and fluorescently tagged DNA building blocks, is added, and the DNA sequence is determined as fluorescence is given off from the building blocks getting used in the sequencing process.

Sequence reading. Each of the four nucleotides is labeled with four different fluorescent tags and the resulting fluorescent signals with their different wavelengths are converted to specific electronic signals. The cascade of the overall reaction with respect to analysis of DNA consists of the following steps: (i) The specific DNA fragment of a pathogen gene, which represents a unique region of the target, is selected as the object of analysis; (ii) The single-round replication of the selected DNA region is initialized. The four nucleotides, adenine (A), thymine (T), cytosine (C) and guanine (G) are labeled with fluorescent tags with four different colors, which are green, yellow, red and blue, respectively, as each nucleotide enters the reaction; (iii) The fluorescent tracers, which have four different colors and emit photons with four distinct wavelengths of light; (iv) A photon with a certain wavelength strikes a light-sensitive material and kicks out a single electron which then instigates an avalanche of millions of electrons in a kind of sparking process within a microvacuum tube; (v) Once it is excited by absorption of a photon, the electron can leap onto the terminal of a single-electron transistor, where it “throws the switch” and is detected. The electronic signal can be measured using an nanoscale electron counter;

Electron Emission-based DNA Sequence Determination. The strategy is derived from a well-known Einstein equation.

The specific DNA fragment of a pathogen gene, which represents a unique region of the target, is selected as the object of analysis.

The single-round replication of the selected DNA region is initialized. The four nucleotides, adenine (A), thymine (T), cytosine (C) and guanine (G), comprise each DNA molecule; Each nucleotide is pre-modified by adding a unique residue that is designed to precisely change the energy level of a hydrogen atom of the nucleotide.

Electrons in a hydrogen atom, in adenine for example, would normally reside in one of the allowed energy levels. If an electron is in the first energy level, it must have exactly −13.6 eV of energy. For an electron to increase its energy level it must absorb light or add a designed residue. With respect to light, electrons absorb or emit light in discrete packets called photons and each photon has a defined energy. The energy that a photon carries depends on its wavelength. Since the photons absorbed or emitted by electrons jumping between the n=1 and n=2 energy levels must have exactly 10.2 eV of energy, the light absorbed or emitted must have a defined wavelength. This wavelength can be found from the following equation, where E is the energy of the photon (in eV), h is Planck's constant (4.14*10⁻¹⁵ eV s) and c is the speed of light (3*10⁸ m/s):
E=hc/λ
Rearranging this equation to find the wavelength gives:
λ=hc/E

If it is in the second energy level, it must have −3.4 eV of energy. An electron in a hydrogen atom cannot have −9 eV, −8 eV or any other value in between. If the electron wants to jump from the first energy level, n=1, to the second energy level, n=2, the electron needs to gain energy. It needs to gain (−3.4)−(−13.6)=10.2 eV of energy to move to the second energy level. If the electron jumps from the second energy level down to the first energy level, it must give off some energy by emitting light.

A photon with an energy of 10.2 eV has a wavelength of 1.21*10⁻⁷ m. So when an electron wants to jump from n=1 to n=2, it must absorb a photon of ultraviolet light. When an electron drops from n=2 to n=1, it emits a photon of ultraviolet light. The step from the second energy level to the third is much smaller. It takes only 1.89 eV of energy for this jump. It takes even less energy to jump from the third energy level to the fourth.

Since DNA is a double strand of complementary single-stranded DNA, each of the four nucleotides in one strand will be complementary to corresponding nucleotides in the parallel strand, adenine always pairs with thymine and guanine with cytosine. The four unique residues that are artificially added to the four nucleotides will emit photons at four different wavelengths when they are paired with their complementary nucleotides on the parallel DNA strand. The energy level can be defined as −13.6 eV, −3.4 eV, −1.51 eV and −0.85 eV.

The energy levels can be measured and will determine which nucleotide has been added and, ultimately, the exact composition of the complete sequence.

Molecular Recognition-based Electron Measurement. It counts the number of electrons which corresponds to the wavelength emitted by each fluorescent tracer.

(i) The counter is integrated in the device by customizing a currently available nanoscale device called a Single Electron Tunneling (SET) transistor. The electron counter has two components: a capacitor and an electrometer for monitoring. The counter is based on seven nanometer-scale tunnel junctions in series;

(ii) The counter “pumps” electrons onto the capacitor with an error rate of less than one electron in 10⁸. The electron pumping is monitored with a SET-based electrometer fabricated on the same chip as the pump, with a charge sensitivity better than 10⁻² electrons; (iii) The capacitor uses microvacuum as the dielectric, resulting in a frequency-independent capacitance. To operate the ECCS (Electron Counting Capacitance Standard) approximately 100 million electrons are placed, one at a time, on the capacitor. The voltage across the capacitor is then measured, resulting in a calibration of the cryogenic capacitor;

(iv) The electronic signals are amplified, the signal interpreter reads electronic pulses generated from the fluorescent colors of the labels, and the DNA sequence is determined as the random reading continues.

4) Molecular Matching Pattern Indication. The turnplate-featured technology uses color to read sequences: (i) the complementary structures of potential target DNA sequences are immobilized in the metrics of microwells. The number of microwells can be from a few to over 10,000 and each can contain one unique DNA sequence; (ii) All microwells are designed to be electronically “excited” when binding of complementary DNA sequences occurs; (iii) Once it is excited by the absorption of a photon which is designed to be resulted from a perfect molecular matching, the electron leaps onto the terminal of the single-electron transistor, where the electronic signal is propagated to the Molecular Recognition-based Electron Counter; (iv) The Counter will localize the signal on the signal emission “map” that describes the precise locations of each microwell and point out which microwell has been excited; (v) The electronic signal will be amplified to reach a readable level. Although target DNA was used in this example in order to describe the technology, the technology can be easily extended to other types of molecules in order to identify bioagents.

24. The CMOS Circuits

The laser lithography technique is based on direct laser writing on substrates coated with a resist bi-layer consisting of poly(methyl methacrylate) (PMMA) on lift-off resist (LOR). Laser writing evaporates the PMMA, exposing the LOR. A resist solvent is used to transfer the pattern down to the substrate. Metal lift-off followed by reactive ion etching will be used for patterning the structural poly-Si layer in the CMOS. A hybrid methodology that combines molecular simulations to perform a classical engineering is chosen to be used. The molecular simulations provide the elastic and/or electrostatic properties of each component of the system considered individually, estimated from the force field, while the classical analysis provides the behavior of the assembled system based on those properties. In summary the steps followed in the present design are the following:

(1) Molecular Simulation Steps: (1) Selection of force field parameters for the elements included in the design; (2) Selection of a monolayer; (3) Evaluation of the Young's modulus of silicon at length scale of the actuator cantilever; (4) Selection of the SWCNT and evaluation of its strain energy function and point of mechanical failure (buckling) in the appropriate range of curvature (for “free-end” designs); (5) Evaluation of the SWCNT crimping energy as a function of the inner opening (for “in-line” designs); (6) Evaluation of the electrostatic energy of the monolayer as a function of cantilever curvature and dimensions for various levels of charge density (pH).

(2) Classical Engineering Steps: (1) Valve assembly and geometry optimization; (2) Evaluation of the mechanical properties of the charged cantilever as a function of curvature and dimensions; (3) Evaluation of the mechanical properties (strain energy and forces) acting within the assembled device as a function of monolayer charge, device geometry and curvature of the components, and determination of ranges of operation as well as equilibrium geometries.

The array of reactor-coated microcantilevers are optimized to have the potential to offer high-throughput detection of proteins, DNA, RNA, peptides and whole-cell. The highly sensitive electron beam lithography (EBL) based micro and nano cantilevers are able to facilitate simultaneous analysis of multiple samples for multiple analytes and improved measurement confidence through increased statistical data.

(1) Design I: The cantilever is a free standing structure with a multilayer of thin films, which consists of a coating layer, a passivation layer, a piezoresistive material layer, and the silicon base. The coating layer will selectively bond with the target molecules, or will carry some biomaterials that bond with target molecules. The resultant surface tension force change will deform the cantilever and the embedded piezoresistive materials. The two legs of the cantilever form a closed loop for measuring the change of the resistance or the voltage applied. The deflection will be monitored. For some molecule which does not have strong surface tension effect after bonding to the coating material, the cantilever could be used to sense the mass change. In this case, only the tip region of the cantilever is covered with the coating material. The mass of molecules bonded at the tip region will have the strongest effect on the bending of the cantilevers.

(2) Design II: The spring is pre-deformed by depositing layers of stress-engineered thin films. For example, changing the chamber pressure during chromium (Cr) sputtering, a tensile or compressive stress can be formed in Cr. A compressive to tensile stress gradient can be formed by the sequential deposition of compressive and tensile Cr layers such that the released structure will assume the desired pre-deformed shape. Advantage: The embedded piezoresistive material for deflection detection eliminates the use of laser beam, which is impractical for nano-scale and array cantilevers. 1-10 Ådeflection has been reported in similar cantilever used in atomic force microscopy. The array design of the cantilevers has the flexibility to incorporate different sizes of cantilevers, thus facilitating simultaneous analysis of multiple samples and improved measurement confidence. Different cantilevers from 20 nm to 40 um wide will be fabricated. The shape of the cantilever will be optimized considering the dimension of the cantilevers (length, width, thickness and the width of the cantilever leg). The deflection of the control cantilevers will be compared against the deflection of the reference cantilevers when antigens bind from a serum containing antigens.

25. The Turnplate for both DNA Extraction and Protein Purification. The turnplate is an automated platform that is built at integrated circuits and coordinated by a central microprocessor. It contains five types of microdevices. (i). The homogenizer that uses a glass beads is coupled to disrupt cellular materials through abrasion. The resulting pulp is used for DNA analysis or protein isolation. (ii) The Single Wafer Rapid Thermal Processor facilitates measurable and well-controlled thermal changes while each reaction chamber turns to be its designated operation. (iii). The reagent suppliers inject solutions into reaction chamber according to a pre-defined time-table. (iv) The reaction chambers host processes of digestion, catalysis, dilution, washing, elution or others. (v) The waste collector sucks solution from the reaction chamber when its port switches over.

DNA Extraction: (1). The sample in small quantity is placed in the “Raw Sample Inlet” and enter “homogenizer”. (2). The lysate is routed into the “Solution Chamber-I” through a filtered gate for neutralization. (3). The lysate is routed into the “Solution Chamber-II” through a filtered gate for dilution. (4). The lysate is routed into the “Solution Chamber-III” through a filtered gate for clean-up. (5). The lysate is routed into the “Solution Chamber-IV” through a filtered gate for elution. (6). Pure DNA is pumped into the “Sample Outlet”. (7). Pure DNA enters the phases of Real-Time PCR for signal generation.

Protein Purification: (1). The sample in small quantity is placed in the “Raw Sample Inlet” and enter “homogenizer”. (2). The lysate is routed into the “Solution Chamber-I” through a filter gate for lysozyme and EDTA. (3). The lysate is routed into the “Solution Chamber-II” through a filtered gate for inactivation of interfering substances. (4). The lysate is routed into the “Solution Chamber-III” through a filtered gate for microsphere-based isolation. (5). The lysate is routed into the “Solution Chamber-IV” through a filtered gate for elution. (6). Candidate proteins are pumped into the “Sample Outlet”. (7). Target protein enters the phase of bioaffinity-based signal generation.

26. The Artificial Nerve Terminals (ANT). ANT is second form of plant GMO detectors that consists of eight components situated at four nodes.

Node-I: Detection Tip and Aspiration Hose. It contains a branch of tips. It contains two types of tips: (1) the tip coated with thin-film membrane and proper reactors that directly interfaces with plant liquid. Nanowire that has sensitive conductivity is implemented under the membrane. Many tips which are made up with different reactors can be used to target different objects or a same object in a time sequence. (2) the tip looks like a microcapillary that sucks small quantity of liquid sample from plant objects within a distance. Nanowire that has sensitive conductivity is joined with each nodes of the polymers. Many tips which are made up with different filtering polymers can be used to obtain different qualities of liquid samples.

Node-II: Sample Filter and Flow Cascade. They are formed as a branch of extendable and flexible pipes. It contains two types of pipes: (1) the pipe in which the two-way optical fibers lie and the light from projected from the Laser station goes through one line of optical fibers and brings back signals from the reactor-coated tip through another line of optical fibers. (2) the pipe in which samples with distinctive physiochemical properties are filtered through polymers, carried by different groups of microsphere and transferred from the capillary tip to another direction based on mechanical, optical, or electrokinetic forces which is involved subsequentially following phases of the movement. Samples are neutralized, digested, step-by-step eliminated in cascaded polymer sections and targeted analytes reach their destination where the reactions of biocatalyst, bioaffinity or hybridization occur.

Node-III: Laser Station and Electric Center: They are two stand-alone units but bridged together through an interface. (1) Laser Station: It projects Laser light through optical fibers to the tips and carries scattered lights back to the station. (2) Electric Center: it monitors events that the nanowire network has encountered and filters signals at the center.

Node-IV: Message Reader and Signal Transmitter. They perform two different tasks based on different mechanisms. (1) Message Reader displays the signals right at the handle. (2) Signal Transmitter: transfer the signals between the device and remote databases through wireless communication.

27. Demonstration of Single Cells in Microfluidic Environments. The types of individual differences contributing to heterogeneity within a disease-related cell or infection-involved organism population can be divided into at least four general classes: genetic differences, biochemical differences, physiological differences, and behavioral differences. Biochemical or behavioral differences might ultimately be traced back to a genetic basis. Even physiological heterogeneity, which may be driven by forces external to the cell or organisms (e.g., metabolic stage, nutrient limitation or the presence of antibiotics), could be viewed in terms of the cell and organism's genetic potential to respond to these forces.

The choice of methodologies us used to explore cellular differences often makes it operationally clear which source of heterogeneity is the subject of investigation. In our molecule profiling systems, genetic heterogeneity is addressed using modified methods such as single cell PCR, fluorescence in situ hybridization (FISH) and Quantum dots (QD), biochemical heterogeneity is measured using enzyme assays or single-cell electrophoretic separations, and behavioral heterogeneity is measured through direct observation of cellular responses to various stimuli. Options of detecting individual cells or organisms are vary according to their genetic, biochemical, physiological, or behavioral properties.

Dynamic cellular phenomena, including protein expression and behavior, substrate uptake, binding and release of individual chemoattractant molecules to cell surface receptors, selective degradation of uniparental DNA within newly formed algal zygotes, bacterivory, and drug efflux, are observed or measured at the single-cell level through our microscale fluorescence staining techniques.

28. Demonstration of Single Molecules in Microfluidic Environments. Single-molecule fluorescence technique holds great promise for biomedical analysis as it offers an ultrasensitive way to measure biological information with both high spatial and temporal resolution. It is able to generate a detectable signal from a minute amount of sample without amplification using the ultrasensitive single-molecule technique.

We have established and optimized various sensitive, specific, high resolution, high-throughput and low-volume analytical methods and probing schemes for detection and quantification of biomolecules such as DNA, RNA, and protein.

Those methods and schemes which have been greatly modified in fit with a microfludic environment or a microchannel network include: (1) Single-DNA detection in a microfluidic platform using molecular beacon probes and fluorescence correlation spectroscopy; (2) Single-protein detection in a microfluidic platform using quantum-dot probes and two-color fluorescence techniques; (3) Single-molecule manipulation in a microchannel using electrokinetic forces; (4) Single-protein measurement using the technology of Self-Replicating Label for Protein Quantification; (5) Single-protein measurement using the technology of Immuno-Detection Amplified by T7 RNA Polymerase; (6) Single-molecule observation based on the Surface Plasmon Resonance; (7) Single-molecule observation based on the Cantilever Oscillation; (8) Single-molecule observation using the technology of Surface-Enhanced Laser Desorption Ionization (SELDI)-ToF MS.

29. Setup of Signal Measurement and Configuration of Light Sources

Samples are placed within a gap of two optical fibers. The incident light from 1^{st} optical fiber is modulated to pass through the samples and the refracted light is detected at the end of 2^{nd} optical fiber.

In an attenuated total reflection (ATR)-type configuration, the chemical transduction system is placed in a region of the optical fiber where the cladding has been stripped off. The incident light is modulated through interactions of the evanescent waves with the chemical transducer. This type of configuration needs a long light-path length, typically 5 cm, which leads to a large size of the sensor head and prevents measurement in a small space.

In a reflection-type configuration, the chemical transducer is placed at the distal end of fiber. The incident light is transported along an optical fiber, encountering the chemical transducer at a terminus of the fiber. The reflected or emitted light by the transducer are collected and carried along the same or a different fiber. However, the light collection efficiency by the detection fiber is low, debasing the sensitivity of the sensor.

The configuration is characterized by an air gap design. The two optical fibers were fixed face to face with each other so that a small air gap existed between the two fibers. One or both end faces of the fibers were coated with a sensing film whose color change was monitored through the fibers. This configuration will reduce the size of sensors and will also reduce the loss of the light transmitted through the sensing region.

30. Miniaturized Laser Setup

The approach is designed for the situation that if the cantilever tips in which samples have attached are brought into a laboratory for analysis. The periodic driving signal with a controlled modulation amplitude can be provided by a 415 nm diode laser, wherein the laser spot can be located at some distance away from the clamped end of the cantilever. The measured resonant response of the cantilever can be obtained at distances in excess of 160 μm with varying oscillator dimensions. The effectiveness of the driving mode will be further studied for different combinations of materials, such as Si—SiO₂ and Si₃N₄—SiO₂. When excited by energy from a laser, these cantilevers oscillate at frequencies of around 11 to 12 Megahertz (MHz). The frequency is measured by shining another laser on the oscillator and noting interference patterns in the beam caused by the reflected light. The change in mass of 1 attogram would be enough to shift the frequency of vibration by 50 Hz or more, depending on the size of the oscillator. A single laser can be used to excite vibrations in nanomechanical oscillators and to measure the resulting vibrations. The excite vibrations can be detected by shining a laser on a spot nearby on the silicon substrate, while reading results with a second, sharply focused laser scanning the cantilevers. This allows us not only to detect the attachment of a single virus, the binding of a DNA molecule, the affinity of a protein, but also to count the number of molecules attached to a single receptor by the total frequency shift.

31. The Piezoelectric Metrics-Based Energy Reservoir

The microgenerator is created in order to establish a self-powered sensing mechanism. It employs direct charging to convert reactor-binding energy into stored electromechanical energy in a piezoelectric unimorph, and employs piezoelectricity to convert the stored electromechanical energy to extractable electrical energy. The microdevice experiences a charge-discharge-vibrate cycle, integrates the energy collected during the charging phase, that enables high power output for a short time during the vibration cycle. The signal from the piezoelectric element is rectified using diodes and stored across an external capacitor. The voltage bias thus realized can be used to drive electronic signals. While optic signals are converted to be electrical signal through the piezoelectric unimorph, a set of OLEDs will be activated and a set of lights which are correspondent to it will be flashed.

32. Miniaturized Organic Light Emitting Diodes and Array of Signal Flashers

The organic layers comprise a hole-injection layer, a hole-transport layer, an emissive layer, and an electron-transport layer. When a sufficient bias is applied to an LED device, electrons and holes are injected respectively from the positive and the negative electrodes into the electroluminescent material. Electrons and holes recombine within the electroluminescent material, forming a neutral excited species—electro luminescence.

The structure of the organic layers and the choice of anode and cathode can be defined to maximize the recombination process in the emissive layer, and thus maximize the light output from the OLED device. Single-layer assembly, an organic or polymer LED contains an electroluminescent material (emitting layer) sandwiched between two electrodes.

Electrons and holes recombine within the electroluminescent material, forming a neutral excited species (termed an exciton). Excitons decay to the ground state liberating energy. A fraction of the liberated energy is in the form of light.

Excitons decay to the ground state liberating energy. A fraction of the liberated energy is in the form of light. The color of the light emitted depends on the difference in energy between the excited and the ground states.

The color of the light emitted depends on the difference in energy between the excited and the ground states. In a first approximation, optimal device efficiency is achieved if the two electrodes possess Fermi levels (or electronic work functions, phi) that closely match respectively the valence (HOMO) and the conduction (LUMO) energy levels of the emitting material. In other words, the Fermi energy of the anode should match the valence band (HOMO) of the emitting material and the Fermi energy of the cathode should match the conduction band (LUMO) of the emitting material.

The signals are initially generated at surface of the microstick. While optic signals are converted to be electrical signal through the “fiber optic data link”, a set of organic light emitting diodes will be activated and a set of lights which are correspondent to it will be flashed.

33. The Fiber Optic Data Link

The fiber optic data link might be implemented in enabling the microsystem for remote communication. The link consists of three parts—transmitter, optical fiber, and receiver.

(1) The transmitter, optical fiber, and receiver perform the basic functions of the fiber optic data link. Each part of the data link is responsible for the successful transfer of the data signal.

(2) The transmitter is needed to effectively convert an electrical input signal to an optical signal and launch the data-containing light down the optical fiber. The receiver is needed to effectively transform this optical signal back into its original form. The electrical signal provided as data output needs to exactly match the electrical signal provided as data input.

(3) The transmitter converts the input signal to an optical signal suitable for transmission. The transmitter consists of two parts, an interface circuit and a source.

34. The Neural-Network Combinatorial Code Board

The Neural-Network algorithm-driven software instructs the workflow of the multiple target recognition and the multiple channel signal reporting. Steps of the workflow: (1) Multiple molecules to be detected in an open environment; (2) Microarray of the molecules featured with distinctive motifs that will cause unique antigen/antibody interaction; (3) The detected molecules react with a built-in enzyme-based reporting system and the chemical reaction triggers electronic signal; (4) Neural-Network-based Pattern Classification; (5) The Combinatorial Code Board for interpreting and classifying the amplified signals; (6) Molecule Recognition and Signal Identification; (7) Signal reading and data reporting through wireless communication.

35. The Parallel Signal Processor

The microprocessor is developed based on the RISC (Reduced Instruction Set Computers) Architecture.

The Neural-Network algorithm-based combinatorial code board is implemented as the core port of parallel signal processor for orchestrating data flow from hundreds of signaling channels simultaneously. It interprets the signals generated, confirms the simples processed, quantifies the level of target molecules and their concentrations, compares the various environmental factors involved in the instance, indicates the probability of detection and false positive, compares data against regional, national or global databases, determines the response time in each of different instances, and triggers proper alarms.

Specifications of the signal processor. (1) Power: One or two milliwatts; high-performance powered by 700 MHz PowerPC 750FX processor; (2) Computation Speed: Up to a mega-flop; (3) I/O: Custom, any sort of hardware; (4) Size: The die sizes can be achieved using current fabrication process technologies (0.25 u, 0.35 u, . . . ). So, depending on the place-and-route tools and the process used, comparable die sizes can be achieved; (5) Cores: Very small, 35K gates; (6) Synthesizable: The microprocessor core is a fully synthesizable design. The design can be ported to the embedded libraries rapidly and be ready for place and route with maximum Time-To-Market efficiency; (7) Interfaces: Optional 10/100/1000 BaseT Ethernet interface can be implemented. Single-width PMC with a 66/33 MHz PCI interface can be implemented; (8) Security: The microprocessor supports popular encryption algorithms; (9) Memory Management: 32 MB of Flash memory and up to 512 MB on-board memory; (10) Interrupt Structure: Custom, efficient, very fast; (11) Operating System Port: A Mac OS runs on a SPARC Station or Windows runs on a PC; (12) Environmental: High Temp, Low EM Emissions; (13) Bytecode Execution: Most bytecodes are handled directly in the hardware. The exceptions are the more complex bytecodes referred to as ‘long’ bytecodes. These are trapped by the processor and emulated by executing software in native RISC mode. Since the decoding of these long bytecodes is still done in the hardware, even long bytecodes are executed efficiently. Also, handling the complex bytecodes in software allows any JVM to be ported to the microprocessor rather than imposing a specific implementation. Java, C and C++ bytecodes all can be executed in hardware; Bytecode Execution Mode: The microprocessor can run in two separate execution (14) modes, native and Java. There is a simple mode switch that changes the execution from one mode to the other. From native mode, there is a single instruction (DISP) that switches the execution to Java. This instruction is single cycle/single byte for simplicity of transitioning modes. In Java mode, when a long bytecode is encountered, it automatically switches to native mode. Because of the mode switch, Java bytecodes execute only when in Java mode and native instructions only execute in native mode; (15) Java bytecode execution: The microprocessor can be operated via co-processor architecture. The core RISC engine has a four-stage pipeline for the native execution mode (Fetch-Decode-Execute-Writeback). When in Java mode, there is an additional stage added to the pipeline that decodes the Java bytecode and translates it to be executed as the native equivalent (Fetch-Decode-Decode-Execute-Writeback); (16) IDE and tools used for developing the microprocessor: The tools suite includes: Integrated Development Environment (IDE), compilers, assembler, linker, source debugger, multiple simulators, development board with ROM emulator, and an in-circuit-emulator (ICE). Each file type in the IDE is brought together in a project and the corresponding compiler is used automatically dependant on the file type. In addition, the source level debugger seamlessly transitions between each language while debugging code. The IDE handles Assembly, Java, and C/C++; (17) JVM, the memory footprint and Java version: The full JVM is not implemented in the hardware. The microprocessor executes the Java bytecode set in hardware only. The microprocessor is supported by JV-Lite virtual machine. It has a memory footprint of 80-90 KB. The virtual machine supports the JDK 1.1 and JDK 1.2.

36. The Modified Techniques Used for Microfabrication.

The microfabrication techniques are used for the construction of Microsystems include silicon micromachining and lithography, chemical etching, laser ablation, photopolymerization, micromolding, and embossing. These processes are optionally used to create the valves, channels, reservoirs, and other discrete microstructures critical to the function of a microsystem and also allow the incorporation of sensing or control elements such as microelectrodes or ion-selective field-effect transistors. A number of players or actuators of microsystems, include pH-responsive hydrogel valves, ferrofluidic micropumps, units of turnplate, pressure-sensitive elements, nanowires, CMOS circuits, and microrobotic “arms” fabricated from conducting polymer bilayers. Microrobotic devices and smart cards, which are capable of manipulating individual micron-scale objects within an aerosol or aqueous environment, are used for the discrete positioning or transfer of individual molecules or cells between analytical chambers within a microsystem.

37. The Customized Materials Used for Microfabrication.

The manipulation of very small quantities of liquids by micropipetting has a lower limit of about 1 μl. Below this value surface forces become too strong to be reliably controlled and small variation in manipulating the pipette change the parameters considerably. In addition the very rapid evaporation of such small amounts of liquid becomes a main concern. As a possible solutions micro capillaries or integrated chips can be used to circumvent these problems also opening the way towards a large scale integration of a many-step protocols. Two main categories of chips are considered: hard and soft chips. The advantage of soft chips made of plastics as PDMS are the simple fabrication, low cost, very good chemical and thermal resistance, and simple and tight fitting of external tubings. We have adapted a well established protocol to realize micron-size proper structures in PDMS using negative photoresist (SU-8) producing a relief structure on a substrate.

One skilled in the art having the benefit of this disclosure will appreciate the far-ranging applications of the present invention. The present invention's 4S-featured biodetection microsystems provide analytical measurement techniques with an ability to constantly identify and quantify bioagents rapidly and cost-effectively. The present invention can be used in numerous types of applications, including within the medical, environmental, industrial, and military sectors. For example, in the medical field, the biosensors of the present invention can be used for research purposes, home diagnostics, and point-of-care evaluation. In the field of medicine, the present invention can be used for patient self-control, home health care (such as monitoring glucose, lactate, creatinine, phenylalanine, and/or histamine levels), in vivo analysis, long-term in vivo control of metabolites and drugs, as a control element (biotic sensor) for artificial prosthesis and organs, for rapid analysis at intensive-care units, for surface imaging of organs during implantation, and for bedside monitoring.

In the field of clinical chemistry, the present invention can be used for diagnostics for metabolites., drugs, enzymes, vitamins, hormones, allergies, infectious diseases, cancer markers, pregnancy and other diagnostics, as well as for laboratory safety.

In the field of environmental protection, the present invention can be used for pollution control. It can also be used for monitoring/screening of toxic compounds in water supplies, solid and liquid wastes, soil and air (e.g. pesticides, inorganic ions, explosives, oils, PAHs, PCBs, microorganisms, volatile vapors, and gases. It can also be used for self control of industrial companies and farms. It can also be used in alarm systems for signaling hazardous conditions. It can also be used for the determination of organic load (BOD).

In the chemical, pharmaceutical, and food industry, the present invention can be used in monitoring and control of fermentation processes and cell cultivation (substrates, metabolites, products). It can also be used in food quality control (screening/detection of microbial contaminations; estimation of freshness, shelf life; olfactory qualities and flavor; rancidity; analysis of fats, proteins, carbohydrates in food). It can be used to stuffy the efficiency of drugs. It can be used for detection of leakage and hazardous concentrations of liquids and gases in buildings and mine shafts. It can be used for indoor air quality checks. It can also be used in the location of oil deposits.

In agriculture, the present invention can be used in applications such as quality control of soils, estimation of degradation/rottage (such as of biodegradable waste, or in wood or plant storage), rapid determination of quality parameters of milk.

The military applications for the present invention include the detection of chemical and biological warfare agents (such as nerve gases, pathogenic bacterial, viruses).

Of course, the present invention contemplates numerous diverse applications in any number of fields. That which has been disclosed herein is merely exemplary. As is clear from this disclosure, the present invention is far-reaching and includes numerous variations and alternatives and is not to be limited by the specific embodiments presented herein. Numerous variations and alternatives are all within the spirit and broad scope of what is claimed.

### REFERENCES

1) Edward Stuebing, “Advanced Aerosol Sampling Technologies for Homeland Security”, ICATHS 2004, 8/2004
2) Dena M. Bravata, Vandana Sundaram, Kathryn M. McDonald, Wendy M. Smith, Herbert Szeto, Mark D. Schleinitz, Douglas K. Owens, “Evaluating detection and diagnostic decision support systems for bioterrorism
3) J. M. Eighenholz, “Integrated packaging and testing of optical MEMS”, Advanced Packaging, 11, 27-29, 2002
4) D. I., Amey, B. E. Taylor, D. M. Horowitz and J. Samuel, “Next-generation packaging for fiber optics and MEMS”, Advanced Packaging, 11, 30-33, 2002
5) M. Robert, “MEMS packaging and microassembly challenges”, Proceedings of SPIE—The International Society for Optical Engineering, 3891, 22-25, 1999
6) Y. Zhou, Y. L. Lam, S. D. Cheng and C. H. Kam, “Step-etched prism coupling for optical waveguide biosensors”, Proceedings of SPIE—The International Society for Optical Engineering, 3491, 1163-1166, 1998
7) J. Backlund, “Multifunctional waveguide grating couplers for integrated optics”, Doktorsavhandlingar vid Chalmers Tekniska Hogskola, 1714, 48, 2001
8) O. Kohls and T. Scheper, “Setup of a fiber optical multisensor-system and its applications in biotechnology”, 70, 121-130, 2000
9) Cheng X, et al J. Vac. Sci. Technol. B, Vol. 19, No. 6, November/December 2001
10) Chen X., Zhu Y., and Wang A. SPIE: Micromachining and Microfabrication Process Technology IX, 1/2004.
11) D. Yeo, R. and S. Yao, Combinatorial Chemistry & High Throughput Screening. 2004, 7(3): 213-221
12) Loreto Mateu and Francesc Moll, Review of Energy Harvesting Technique and applications for microelectronics, 2005 http://pmos.upc.es/blues/publications/RevEnerHarvMicro.pdf
13) Amit Lal, Rajesh Duggirala and Hui Li, A Radioisotope-Powered Piezoelectric Generator, January-March 2005 (Vol. 4, No. 1) pp. 53-6
14) A. Curioni and W. Andreoni. IBM J. of Research & Development Vol. 45, Nov/2001
15) William Stallings. Computer Organization and Architecture. 6^{th} Edition. 2003
16) “The silicon guinea pig”. MIT Technology Review. 06/2004
17) Stroh, H. Wang, R. Bash, B. Ashcroft, J. Nelson, H. Gruber, D. Lohr, S. M. Lindsay, and P. Hinterdorfer, Single-molecule recognition imaging microscopy, PNAS 2004 101: 12503-12507
18) Sadik Hafizovic, Diego Barrettino, Tormod Volden, Jan Sedivy, Kay-Uwe Kirstein, Oliver Brand and Andreas Hierlemann, PNAS 2004 Vol. 101, No. 49, 17011-17015
19) R. Amirtharajah and A. P. Chandrakasan, “Self-powered signal processing using vibration-based power generation,” IEEE Journal of Solid State Circuits, vol. 33, no.5 May, 1998.
20) K. Bult et al., “Low power systems for wireless microsensors,” IEEE/ACM International Symposium on Low Power Electronics and Design, August, 1996.
21) Stuart J. Roy, Tracey A. Cuin and Roger A. Leigh, The Plant Journal, Vol. 34 Issue 4. 5/2003
22) Han, J., Craighead, H. G. (2000) Science Science 288, 1026-1029
23) Kartalov, E. P., Quake, S. R. (2004) Nucleic Acids Research 32, 2873-2879

What is claimed is:

1. A stand-alone microsystem adapted for performing combinatorial detection of bioagents at a single molecule level wherein the microsystem comprises a three-dimensional microarray component and a multi-layer microfluidics component to thereby provide high throughput screening and high content screening sufficient to allow for substantially real-time performance of the microsystem.

2. The microsystem of claim 1 wherein the microarray component and the microfluidics component are integrated using microsticks-in-column to thereby permit rapid circulation, completed reagent recycling and continuous functioning of the microsystem.

3. The microsystem of claim 1 wherein the microarray component and the microfluidics component are integrated using microspheres-in-column to thereby permit rapid circulation, completed reagent recycling and continuous functioning of the microsystem.

4. The microsystem of claim 1 wherein the microarray component and the microfluidics component are integrated using microspacers-in-column to thereby permit rapid circulation, completed reagent recycling and continuous functioning of the microsystem.

5. The microsystem of claim 1 wherein the bioagent is from the set consisting of DNA, RNA, a protein, a bacterium, and a virus.

6. The microsystem of claim 1 further comprising a sample collection interface operatively connected to the microfluidics component.

7. The microsystem of claim 6 wherein the sample collection interface is from a set consisting of a mini-syringe for scaled collection of liquid, a mini-pressure hose for volumetric breath of air, a mini-screw for penetration of solid matter, and a pin-tip for scratching of an object surface.

8. The microsystem of claim 6 further comprising a signal processing component having at least one mode of operation and operatively connected to the microarray component.

9. The microsystem of claim 8 further comprising a data reporting component operatively connected to the signal processing component.

10. The microsystem of claim 1 wherein the microsystem comprises a dual mode architecture adapted for simultaneously performing both genomic testing and proteomic testing to thereby reduce false negative and false positive results.

11. The microsystem of claim 1 wherein the bioagent is a cell and wherein the microsystem is adapted to provide for fractional separation and parallel sampling of content of the cell to thereby enable observation of functional related cellular entities and related molecules.

12. The microsystem of claim 1 wherein the bioagent is a plant cell and wherein the microsystem is adapted to measure transgenic materials of the plant cell.

13. A universal platform adapted for performing combinatorial detection of bioagents at a single molecule level wherein the system comprises:
a sample collection interface for collecting a sample;
a microfluidics component operatively connected to the sample collection interface;
a microarray component operatively connected to the microfluidics component;
a signal processing component having at least one mode operatively connected to the microarray component; and
a data reporting component operatively connected to the signal processing component.

14. The universal platform of claim 13 wherein the microfluidics component provides for molecule separation of the sample to a single cell or molecule.

15. The universal platform of claim 14 wherein the signal processing component provides for a cascaded process of signal amplification from weak-level molecule-molecule interaction to medium-level fluorescence generation to high-level optical/electronic conversion.

16. The universal platform of claim 14 wherein the data reporting component is adapted for digital reporting.

17. The universal platform of claim 13, wherein the microfluidics component, the microarray component, the signal processing component, and the data reporting component being implemented in a microsystem having a configuration from the set consisting of (a) a dual-mode genomic and proteomic testing configuration, (b) a clinic diagnostic configuration, (c) an E. coli detection configuration, (d) a virus identification configuration, (e) a food inspection configuration, (f) a pharmaceutical screening configuration, (g) an interior aerosol monitoring configuration, (h) an exterior aerosol monitoring configuration, (i) an odorant detection configuration, (j) a poison detection configuration, (k) a diet measurement configuration, (l) an explosives detection configuration, (m) a human smell detection configuration, (n) a forensic detection configuration, (O) a GMO detection configuration, (p) a warzone inspection configuration, (q) an underwater surveillance configuration, and (r) an open environment configuration.

18. A microsystem, comprising:
a sample selection and collection subsystem;
a sample separation and diffusion subsystem operatively connected to the sample selection and collection subsystem;
a detection and signaling subsystem operatively connected to the sample separation and diffusion subsystem; and
a data reporting subsystem operatively connected to the detection and signaling subsystem.

19. The microsystem of claim 18 wherein the sample selection and collection subsystem is adapted for raw plant sample collection and cellular extraction.

20. A method for detection of bioagents at a single molecule level or organism level, comprising:
providing a reconfigurable microsystem adapted for performing combinatorial detection of bioagents at a single molecule level;
reconfiguring the reconfigurable microsystem for an environment.

21. The method of claim 20 wherein the reconfigurable microsystem comprises a microarray component and a microfluidics component integrated with the microarray component.

22. The method of claim 20 wherein the microsystem further comprises a sample collection interface operative connected to the microfluidics component.

23. The method of claim 20 wherein the step of reconfiguring includes reconfiguring the sample collection interface for the environment.

24. A dual mode, genomic and proteomic, method for detection of transgenic material from within a plant, comprising: collecting a sample of the plant using a sample collector; retaining plant cells from the sample while removing portions of the sample which are not plant cells using microfluidics and at least one microarray; and measuring transgenic materials in the plant cells.
